SEQUENCE LISTING (39 sequences)

SEQ ID NO: 1 - DNA, 2195 bp, homo sapiens
agaaagagattaccagccacagacgggtcatgagcgcggtattactgctggccctcctggggttcatcctcccactgccaggagtgcaggcgctgctctgccagtttgggacagttcagcatgtgtggaaggtgtccgacctgccccggcaatggacccctaagaacaccagctgcgacagcggcttggggtgccaggacacgttgatgctcattgagagcggaccccaagtgagcctggtgctctccaagggctgcacggaggccaaggaccaggagccccgcgtcactgagcaccggatgggccccggcctctccctgatctcctacaccttcgtgtgccgccaggaggacttctgcaacaacctcgttaactccctcccgctttgggccccacagcccccagcagacccaggatccttgaggtgcccagtctgcttgtctatggaaggctgtctggaggggacaacagaagagatctgccccaaggggaccacacactgttatgatggcctcctcaggctcaggggaggaggcatcttctccaatctgagagtccagggatgcatgccccagccagtttgcaacctgctcaatgggacacaggaaattgggcccgtgggtatgactgagaactgcgatatgaaagattttctgacctgtcatcgggggaccaccattatgacacacggaaacttggctcaagaacccactgattggaccacatcgaataccgagatgtgcgaggtggggcaggtgtgtcaggagacgctgctgctcctagatgtaggactcacatcaaccctggtggggacaaaaggctgcagcactgttggggctcaaaattcccagaagaccaccatccactcagcccctcctggggtgcttgtggcctcctatacccacttctgctcctcggacctgtgcaatagtgccagcagcagcagcgttctgctgaactccctccctcctcaagctgcccctgtcccaggagaccggcagtgtcctacctgtgtgcagccccttggaacctgttcaagtggctccccccgaatgacctgccccaggggcgccactcattgttatgatgggtacattcatctctcaggaggtgggctgtccaccaaaatgagcattcagggctgcgtggcccaaccttccagcttcttgttgaaccacaccagacaaatcgggatcttctctgcgcgtgagaagcgtgatgtgcagcctcctgcctctcagcatgagggaggtggggctgagggcctggagtctctcacttggggggtggggctggcactggccccagcgctgtggtggggagtggtttgcccttcctgctaactctattacccccacgattcttcaccgctgctgaccacccacactcaacctccctctgacctcataacctaatggccttggacaccagattctttcccattctgtccatgaatcatcttccccacacacaatcattcatatctactcacctaacagcaacactggggagagcctggagcatccggacttgccctatgggagaggggacgctggaggagtggctgcatgtatctgataatacagaccctgtcctttctcccagtgctgggatttctccatgtgagggggcagcaggacacccagggatctagcgtgggggaggagaggagcctaatgagaaaatgaccatctaaagcctgcccttcattggtctggttcacgtctccaaaccagcttggatggtagcagagacttcagggtgctccagccaaacgtatttgggcatcaccatgacctgggaggggaagatgcactgagacgtatgaggcttccagcctagcagccagggccctagcacaaacaggaggctcgccccatctgagcaactgcaggagaggttagtacagtcatgcattgcttaacgacagggacgtgtcgttagaaatgtgtcgttaggtgattttatgaccataggaacattgtagcgtgcacttacaccaacccagatggtacagcccaatacacacccaggatggacgctagagtcgactgctcctaggctacaagcctgcagtgcatgttatggtgtgaatactgcaggcaatcttaacaccacggcaagtatttgtgcatctacacacatctaaacatagaaaaggtacagcataaatacactattgtcatctcagcaga

SEQ ID NO: 2 - DNA, 5857 bp, homo sapiens
ctttcctagagtctctgaagccacagatctcttaagaactttctgtctccaaaccgtggctgctcgataaatcagacagaacagttaatcctcaatttaagcctgatctaacccctagaaacagcttccacgtgtgagtgagaacatcacagatatagaacaatggaagtgacaacaagattgacatggaatgatgaaaatcatctgcgcaagctgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacatgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgatatcaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaattatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgcaaaaaaaaaaaaaaaaaataagaaagaacaaaacaaaccccacaaaaattagctgggtatgatggcacgtgcctgtagtcccagttactcaggatgattgattgagccttggaggtggaggctacagtgagctgagattgtgccactgtactctagccagggagaaagagtgagatcctggctcaaaaaaaccaaataaaacaaaacaaacaaacgaaaaacagaaaggaagactgaaagagaatgaaaagctggggagaggaaataaaaataaagaaggaagagtgtttcatttatatctgaatgaaaatatgaatgactctaagtaattgaattaattaaaatgagccaacttttttttaacaatttacattttatttctatgggaaaaaataaatattcctcttctaacaaacccatgcttgattttcattaattgaattccaaatcatcctagccatgtgtccttccatttaggttactggggcaaatcagtaagaaagttcttatatttatgctccaaataattctgaagtcctcttactagctgtgaaagctagtactattaagaaagaaaacaaaattcccaaaagatagctttcacttttttttttccttaaagacttcctaattctcttctccaaattcttagtcttcttcaaaataatatgctttggttcaatagttatccacattctgacagtctaatttagttttaatcagaattatactcatcttttgggtagtcatagatattaagaaagcaagagtttcttatgtccagttatggaatatttcctaaagcaaggctgcaggtgaagttgtgctcaagtgaatgttcaggagacacaattcagtggaagaaattaagtctttaaaaaagacctaggaataggagaaccatggaaattgaggaggtaggcctacaagtagatattgggaacaaaattagagaggcaaccagaaaaagttattttaggctcaccagagttgttcttattgcacagtaacacaccaatataccaaaacagcaggtattgcagtagagaaagagtttaataattgaatggcagaaaaatgaggaaggttgaggaaacctcaaatctacctccctgctgagtctaagtttaggatttttaagagaaaggcaggtaaggtgctgaaggtctggagctgctgatttgttggggtatagggaatgaaatgaaacatacagagatgaaaactggaagtttttttttgtttgttttgttttttttttgttgttgtttttttttttttttgtttttttgctgagtcaattccttggagggggtcttcagactgactggtgtcagcagacccatgggattccaagatctggaaaactttttagatagaaacttgatgtttcttaacgttacatatattatcttatagaaataactaagggaagttagtgccttgtgaccacatctatgtgacttttaggcagtaagaaactataaggaaaggagctaacagtcatgctgtaagtagctacagggaattggcttaaagggcaagttggttagtacttagctgtgtttttattcaaagtctacattttatgtagtggttaatgtttgctgttcattaggatggtttcacagttaccatacaaatgtagaagcaacaggtccaaaaagtagggcatgattttctccatgtaatccagggagaaaacaagccatgaccattgttggttgggagactgaaggtgattgaaggttcaccatcatcctcaccaacttttgggccataattcacccaaccctttggtggagcctgaaaaaaatctgggcagaatgtaggacttctttattttgtttaaaggggtaacacagagtgcccttatgaaggagttggagatcctgcaaggaagagaaggagtgaaggagagatcaagagagagaaacaatgaggaacatttcatttgacccaacatcctttaggagcataaatgttgacactaagttatcccttttgtgctaaaatggacagtattggcaaaatgataccacaacttcttattctctggctctatattgctttggaaacacttaaacatcaaatggagttaaatacatatttgaaatttaggttaggaaatattggtgaggaggcctcaaaaagggggaaacatcttttgtctgggaggatattttccattttgtggatttccctgatctttttctaccaccctgaggggtggtgggaattatcattttgctacattttagaggtcatccaggatttttgaaactttacattctttacggttaagcaagatgtacagctcagtcaaagacactaaattcttcttagaaaaatagtgctaaggagtatagcagatgacctatatgtgtgttggctgggagaatatcatcttaaagtgagagtgatgttgtggagacagttgaaatgtcaatgctagagcctctgtggtgtgaatgggcacgttaggttgttgcattagaaagtgactgtttctgacagaaatttgtagctttgtgcaaactcacccaccatctacctcaataaaatatagagaaaagaaaaatagagcagtttgagttctatgaggtatgcaggcccagagagacataagtatgttcctttagtcttgcttcctgtgtgccacactgcccctccacaaccatagctgggggcaattgtttaaagtcattttgttcccgactagctgccttgcacattatcttcattttcctggaatttgatacagagagcaatttatagccaattgatagcttatgctgtttcaatgtaaattcgtggtaaataacttaggaactgcctcttctttttctttgaaaacctacttataactgttgctaataagaatgtgtattgttcaggacaacttgtctccatacagttgggttgtaaccctcatgcttggcccaaataaactctctacttatatcagtttttcctacacttcttccttttaggtcaacaataccaagaggggttactgtgctgggtaatgtgtaaacttgtgtcttgtttagaaagataaatttaaagactatcacattgctttttcataaaacaagacaggtctacaattaatttattttgacgcaaattgataggggggccaagtaagccccatatgcttaatgatcagctgatgaataatcatctcctagcaacataactcaatctaatgctaaggtacccacaagatggcaaggctgatcaaagtcgtcatggaatcctgcaaccaaaagccatgggaatttggaagccctcaaatcccattcctaatctgatgagtctatggaccaatttgtggaggacagtagattaaatagatctgatttttgccatcaatgtaaggaggataaaaacttgcataccaattgtacacccttgcaaaatctttctctgatgttggagaaaatgggccagtgagatcatggatatagaagtacagtcaatgttcagctgtaccctcccacaatcccacttccttcctcaacacaattcaaacaaatagactcagactgtttcaggctccaggacaggaagtgcagtgtaggcaaaattgcaaaaattgagggcacaggggtggaggtgggggggttgaataacaagctgtgctaaataattacgtgtaaatatattttttcatttttaaaaattgatttcttttgcacattccatgacaatatatgtcacatttttaaaataaatgcaaagaagcatacatccaa

SEQ ID NO: 3 - DNA, 4302 bp, homo sapiens
aactgaaaatccacaagacagaatagccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaaaaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatattagttgtgttcaacaattagtgaaacagaatgtgtgtatgcatgtaagaaagagaaatcatttgtatgagtgctatgtagtagagaaaaaatgttagttaactttgtaggaaataaaacattggacttacactaaatgtttaattcattcattttattgtgaaataaaaataaaatccttagctcctccaccaactgaacagaccctcttggccaaggagaccccagaaaccttaaaaactaagtttcccaaccatgacaagatgagagatcattcacacctcattatattccctcccttgctaactgccattggactttttccactgagttaaacagaaacccatggaaaacaaagaacagaagactcactccttggctgacttcacctagctcactccacgtagcgccacagccagactcccctcccctcttgcggtttccacatgacaactgatcagccttccctcctgataagtgaccactgcccacagactggttctggccagtccatggaggctgcacacagggtgcctctatgtcctttgtttcaccttttgatatagaaaggctaattttgctgtattttaatgttaagtctccaccacagagtgaacacagaatgcatgtgacatacatgtttacataccactattgtgtgactgcccctcatgaatattcatagccccccataacctgttaactatgtgtgtctagccaatccaccaaccataaaacttctgtaataccctcccttcctccaagagcctgcttttggttgctgtggtaggctctgcttcccaggctgcaggttgcaggagaggaggctgcagtggctcacgcctgtaatctcagcacttcgatgggacgaggcaggcagatcacctgaacccaggagttcgagagcagccttggcaatggcaaaaccaaccgtctctacaaaaaatgcaaaaacttagctgggtgtggtggcatgcacctgtagcttcagttccagctactcaggaggctgaggtgagtggactgctggagccagggagttcgaggctgcagtgtcgagatcttgccactgcactccattctggatgatagaacgagaccccatctcaaaaaaaaaaaaagttctctccaattgtatatagcttgtgattttatgtcaacactatcaataaatagctttcagtgcaagaaaccaaaaatactgtaataaacaggcacatattcttcccaaacctcatgcagtttacaatctagtgagagacacagatagcagtacagagtcaattaaaggttagttttcttcatgaagatgttttaattttaattcaatgtgaaagggttccaaggagtttatcttgttttatgccattttatttgaagcactacttactaagtcatttgctgatattaatctagttaaatcaagaaatattacatgaaaatgttgctaaatcagagatcatgggtaacaatcacctttgattatgaataatcatattttattgaaaggcaaggcacaacaaataataagaaggaaaaaataaataagcaatgttattgatctttcattctgtatatgttttggggggaatatactagtttcttttagtggctgtaacaaattaccacaaacttggtgacttaaaatttcacagatttactctttcttacagttctggaggtcagaagtctgaaatgggtttcaatgagccaaagtcaaggtattgatgacgctacactcctccggaggctctaggcagatagccttttccagcttccagaggctgcctgaattctttcatccatcttaaaaaccaacagtgtagtagcctcaaatctctctctctgcttccttcttcacatctccttctctcctctgactcttttgcctctttcttctaaggacgcaccaggtccacctgcataatccagaataattgccccatccgcaaatccttaatttaataacatctgcaaagtcccttttgctatgtaaagtagcatgttcacaggttctggagacttggccatggatacgattgcggggggggcattattcttaccacagagcaccccaagaaaatctccaaattttgggcttccaatccattttgcttcaattatttaatatttttactccttccagtagatactgatttcatccattgcccttaagaaggtaggacagagattatggcacatctcacattaaatgctatattttcgttggaaatacattttttgcttcaacttttattttaaattcaagggtacatgtgcaggatgttcaggtttgttacacaggtaaacgtgtgccatggcggtttgctgaacagatcatcccatcaccaacagatcatcccattgagaggtgaagccggctgggcttctgggttgggtggggacttggagaacttttctgtctagctaaagtattgtaaaatggaccagtcaacactctgtaaaatggaccaatcagctctctgtaaaatggaccaatcagcaggatgtgggtggggccaagtaagggaataaaagcaggccacccgagctggcagcggcaacccgctcgggtccccttccatgctgtggaagttttgttctttcgctctttcaataaatcttgctgctgctca

SEQ ID NO: 4 - DNA, 2336 bp, homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttta

SEQ ID NO: 5 - DNA, 551 bp, homo sapiens
gccaaacaccttcctgacaccatgagggccagcagcttcttgatcgtggtggtgttcctcatcgctgggacgctggttctagaggcagctgtcacgggagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctcca

SEQ ID NO: 6 - DNA, 4888 bp, homo sapiens
ctggcctccgccgcgcgctcctcctcgtcccagcgctagcgggcacgcggttcctttttgcgagctttccgagtgccaggcgccggccggctgcgaagacgcggtgggccgcccctccgattgaaatcacagaagatattcgtgttcttcttaagagaaaaagaggacatttaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaataaaatgttgttgtcttattgacagttgtgcaggaggtagcctggtggttttcaacctctagaattttaagcctttgttgaactgttagaatgtaaggtatatcattctaaagatagagtaaaaagaaaacaaaaccaaaagttattaaaattgttgtccggtttactttaacttagttttgcatagttctagtgcagctgaaattgaaaagttatttccctttagctgtgttattatagagcagaaattctgtttttaaaaattagcctaagatatacttgtttttgtaaagaaaaatatttaatgttgaacaaaataaattggagttggagtagaatgtagtttgaggaaatttgcagcttccaatgcctcttgtcttcctatttcagaagtttaaatattaagcatgacagaaaatatgtattaacactactcaaagcaaaagtgctgcagggctttaaaattctcttccaaccatttatcttgaaggaaaaattcaatagtaatataatacacaaaatcaaataataccttagaaggtattaagattataattgttgcataggttagatatagagtcattgtaatgttgtgaataattacagtgcctaaaataagaatagaacaacatatacaacaccaaaaaatatctagtaatatatttaaagggaaattgagctgctttttttgaaactttgagatctaaaaataactgtaattatttgaatgactaagaggaaagtacattttttgaaatgctgaaaattgcctttctgtgtttattcaaactgaaaagctgagaccaagagcaaggaaggtaaaaagttaacaggcaaacattttctcttagaaaaggtgataaaatcataagtatttggaattagaacccttgcacagcactgaacctgggaaagagatttaaactctgaatttatctttgataacagggattgattttaaaatgtacatgtattaaattacatttgtaatttaaggtctgtttgctgttgctgattttattcttgatcagtagtttgcatttcagaaagcctttcattttgctttaattttagcaaagcgggttataatgaatgacttccccaatatcttgcttgaacttacaggtgattaacttggatgagttttgggaagttaaagggaagaaaacactgttatcattttttcctgtttgggaagagcttagaaactggaaatactagatttgggagaagggcagagttacttgataagggactgatgtttgtgcagtaacttgggagtgtggtttctttttgaatctttaattaaaacctgggattatatatccctgataaatattcacacttgaaccatagttactgtaaaatgcaaaaaatcttaatactgttattctttgcactttttcttaatcattttttatatatatgcatatatatatgtgtgtgtgtgtgttgcttatgttgttttgtacagatgtgggccaccattgcaacaaaatacattctttttgctctaaaatatttatgaagaaaatacttaaatgttatgtatatggtggtaataagggaaaaatcaagtattataaacaagaatgaaggtttttgtaaagatttctgttcagcgttttgcaaggtaaaattttaggcaagttttccctgaagttatgtgtatgtgagtattctcattcttcccaacttgcctttgaagagtgaaataccattattatcaagtagactactgttcagcttttattcctgccctgctgtttatcccttaagaatgagtttcttagacttttccaatatgtgattttttttcccatttagaatggtgattttaaatgtgtgagtgcatgtactatcttatctcagatatttgcacccccaatctgcccccaactcccaaaagctagaacactgccaactgatctgttataggtcctttagaaacacataattaacacttaaggttgggtgctgctaattctttgcaaaaatccaaatattgttaagggaccagggagatgccactaccccttgattttccatctaaaaatatacatgtttatgtaaacaaatctttccatatccatagtgacttttcaagtatttaagcctaaagattttgatctcacatttttatacctgtttaaattgctcacagttattacatacacatcagccatcaactaaagttgtactttaaaaatttactacaatatgtacatttctaagtcaaacacttgtgacttttgctttaattccatgaatgttcctgcctccttgatatttgtatttattctttttttctctagagtagaggtataattgtgtgatatttcagaaatacagataaatgattcaaaaagtcacagttaaggagaatcatgtttctttgatcatgaataactgattagtaagtcttgcctatattttcctgatagcatatgacaaatgtttctaaggtaacaagatgagaacagataaagattgtgtggtgttttggatttggagagaaatattttaatttttaaatgcagttacaaattataatgtattcatatttgtactttctgttaaaatgcatgattgcagaattgtttagattttgtgtttattcttgatgaaaagctttgtttgttcttgtttttaagtttgcactcaaatcttaagaaataaatccacccatgttatcaa

SEQ ID NO: 7 - DNA, 2536 bp, homo sapiens
gccgcgttgcgctcccgccactccgcgcccgctatcctggctccgtgctcccacgcgcttgtgcctggacggaccctcgccagtgctctgcgcaggattggaacatcagttaacatctgaccactgccagcccaccccctcccacccacgtcgattgcatctctgggctccagggataaagcaggtcttggggtgcaccatgatttcaccattcttagtactggccattggcacctgccttactaactccttagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagtgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctgctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcggcaggcagccttgctgcaggccccctgctgctcgcgctggccctctaccccctgagcgtcctgttctgagggcccagggcccgggcacccacaagcccgtgacagatgccaacttcccacacggcagcccccccctcaaggggcagggaggtgggggcctcctcagcctctgcaactgcaagaaaggggacccaagaaaccaaagtctgccgcccacctcgctcccctctggaatcttccccaagggccaaacccacttctggcctccagcctttgctccctccccgctgccctttggccaacagggtagatttctcttgggcaggcagagagtacagactgcagacattctcaaagcctcttatttttctagcgaacgtatttctccagacccagaggccctgaagcctccgtggaacattctggatctgaccctcccagtctcatctcctgaccctcccactcccatctccttacctctggaaccccccaggccctacaatgctcatgtccctgtccccaggcccagccctccttcaggggagttgaggtctttctcctcaggacaaggccttgctcactcactcactccaagaccaccagggtcccaggaagccggtgcctgggtggccatcctacccagcgtggcccaggccgggaagagccacctggcagggctcacactcctgggctctgaacacacacgccagctcctctctgaagcgactctcctgtttggaacggcaaaaaaaaatttttttttctctttttggtggtggttaaaagggaacacaaaacatttaaataaaactttccaaatatttccgagga

SEQ ID NO: 8 - DNA, 1471 bp, homo sapiens
gtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagtaactcaaaactttttaattttagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaaga

SEQ ID NO: 9 - DNA, 6147 bp, homo sapiens
actgtgtccgggtcagctgctgccgccgacgccgtaccgctgcggccgggggattgggccggggtctccaccgccgaccgaggggagcgggctccgctcggccctgctttttgcgacctggccgtcagccccacgtcgccggcctggaggggcgaagaggacgagggggccaaggcttcctccggggacattggctccctggattatcaagagtttgtagttgacattgaatccaggctgaggatggaaggtgtggaacttaaagaagaatggcaagatgaagattttccgatacctttaccagaagatgatagtattgaagcagatatactagctataactggaccagaggaccagcctggctcactagaagttaatggaaataaagtgagaaagaaactaatggctccagacattagcctgacactggatcctagtgatggctctgtattgtcagatgatttggatgaaagtggggagattgacttagatggcttagacacaccgtcagagaatagtaatgagtttgagtgggaagatgatcttccaaaacccaagactactgaagtaattaggaaaggctcaattactgaatacacagcagcagaggaaaaagaagatggacgacgctggcgtatgttcaggattggagaacaggaccacagggttgatatgaaggcaattgaaccctataaaaaagttatcagccatgggggatattatggggatggattaaatgccattgttgtgtttgctgtctgtttcatgcctgaaagtagtcagcctaactatagatacctgatggacaatctttttaaatatgttattggcactttggagctattagtagcagaaaactacatgatagtttatttaaatggtgcaacaactcgaagaaaaatgcccagtctgggatggctcaggaaatgttatcagcaaattgatagaaggttacggaaaaatctaaaatccctaatcattgtacatccttcttggtttatcagaacacttctggctgttacaagaccatttattagctcgaaattcagccaaaaaattagatacgtgtttaatttggcagaactagcagaacttgtccccatggaatacgttggcataccagaatgcataaaacagtatgaagaagaaaagtttaaaaagaaacaaaaaagagttgatcaagaacttaatggaaaacaagatgaaccgaaaaatgaacagtaagtttggcatctagtccaaacaagactgaagaatgtgctgatggagcagtgctgtttctgcattcataatgcatttattggcccatatttttatgtaacctgttacaaaatagacttgactttttcataatggacttttgtattatacaagggactgttcactgctgtactggtttgcaaatttcttgaatttagctctttaatagctaactgtattattatcgttttatattttatattgctaaatagagaaccacactttatataaagtagtttttgcatttgtttattgaatgatgcatcttcttcggtgaaatatttatatgcataaatggcaaaggaaagaaataatatatatttttatgtcattgagcaatattttttaatgtgtacctgtcttatggaagaaatatgcaggtatataagaccacgattttctaaagtgcatattagaatttttgtttttgtaaatggttaaatacatttcctgggtaacttaggaaattaagtttttcataaggcaacagatggttaaactgattgtcatgaatacccaaagatcatgtatataatcgaagtgtattagtaccatcccaaggtttttttctcatttaacatatttgtttcataattcagcaagtacagatgcaagcgcattgcacactttttcctttctaaacttaaagacaagtcaaaaagccattcttagaactagaggatttaagagggtaggaattagggtttgtatatatgtatatatgggacattttatcttctggcccaaagtcagaactttataaaaatcttgagtttgttcactaatgtgaaataagctatgtgtccagggtattgctcccctgagtgtatatgagtgctgagtagtattgcagagaatgtgatgagttattcactgtcacaactttttctatagaaaacaggggctgctttttaaactctcactatgggcactttaccaaaatacttccatatcaattatttgaactcggtagttgtttgacctagttagatgtggtgtttattcaagtttgtatgaaatcatgtttgacaatactgtaaattaggttaattttgtaagtcttagcatcatcatattgtgctgttttggataacacgtttgttacaagcatttaaactgtttcatttggtagtacctttacattgaaataaattgtgtttgtgcctccaaacaaaaagtttaattaccgacaaaattatttctaaaacaacagattgatctgatattgtgtcatatacttgaaaggctcatacacagctggtaagcataataccttccaaatttgaaggtcatggctactcttcaacccttcctgtaaggtattttacaaaacagtggcctagagtagcaaaattgaagtggatgtgagctaacctttcaagatttagtctatcctagattgaacacacatgtggctgtgaccattctcatgtacttacaaaccttctgttacaaaagggaactttcctatattttttgtcctcctcagtcctaaacttggcatgctaacttaaaaccagttataggagagagatgctggaggattcccaggatgaaacattcctcagcagcctcctgacttaactcagatccagcagctcttggcagatcctgaactaatgtcattacagtgaattgagccaaaacgattattgcttgctcacattttaggaacacttgttgttgaagaagcaaactaacaataacaagttaggcattccttctgaatttaccatataaggattatgtataaacaattatattgaatttgtcattagattctatatttgtagacgatttaacgtagtctttcatctgtacatctttatcttgctcaaaatgggctgaggtttttaataggtggttttctaccacattaagtacttggttcttcagtttaaaaaatatgaagaataacttcaactcttttttgcattattttgatttttgattttgcagtcattgctcctaactgtagatacatgttgcaaaagtgatgtttttctgaattttttttaaaagtagaagactattctgttaaacattaaaattcagagacaacagtgataacacctgggatataatgaaaatgaggtgctgtgaatcagtaaactaaagggatacctgctttaacctgcaccttaatggaatctggtatagagatgtacccacaataaatgttttataagatttaagatcataatagtaggttagaggaagagcatttattaagtctgttgtttctaatctatagctaagttaacatttcccatattattccattttaatttcccattactgaccaaggtgaaagtatatgtgattgtacttttatctttctgttgggatcatggatctaatttaactggcttaatttgatgcttctgtcactaggctctaagtgccccttttttagtcagaagtttttcatgtgaaataatttgattctgtgttctgcttgatcatactcctaagcaagcaccaagggaagacgttctattcaccccacaatttaagcttaacaatctttaactcagagggagatatgaagaaatatgattctaagtagggtctagcagaggcattggaaagtgataaaagtaggaagtaagagaaagcaaacctttataaagagataaagacatagttgttacctaagatgccactgtatgtgtgccctctctttttttttttttttttttggagacagagactcactctgtcacccattctggagtgcagtggcgcaatcctggctcactgcaacctctgcctcccgggttcaagcatttctcctgcctcagcctcctgagtagctgggattacaggcacacgccacaatgcccggctaatttttgtatttttagtagagacggggtttcgccatgctaggctggtctcgaactcctgacctcatgatctgcctgtctcagcctcccaaatgcttggattacaggcttgagccaccacgcccggccatgtgtgtccttttaagggaagtaccatatgtgactgctgatggtaacatttcttgtatgcagcaggaaaacctgcaggctatacgcaaggtatgtgaataaagactgtggattgaagggggaaaagttgataatacatacccatgataagaattcatattttctctttctttgtctcttcatacagaagcaagttcgagcagagaaggttagcagttacagcacttaaaagtttaggattgagggtttttggttggtttttttgttttttcatatttcactgttgctgaacattgatttagtcttccaaaatggacatcactagtaagtgcactgaatactgaaatatgctgattggcaagcatatatctgtagctagaatttttttaaccgtaattccagaatttaaaaaggtaagagatctgctttttgatagaggccttttaaaaagactatataacatttctatttggctgtttatgtgaatgctcagggaatttggacatgtgttttatacagaagtggctttttatatttcttcaagtgataaattagcattaggacatatcagatagaacaggccagtttcctgatgagattggtagagatgttacggtaggttcatccttcctttttccatgttatatgtggagaagtgttaaacttgctcaagcaaatgggagtaggagataggctgaaaatagagattgctacaccattctattttgttttcatgcctgttttttctccaatcctatatggacatttgagaaatggaccacaaatctgagagggatagagtaaaaagaggcattaatgtatgtactcacaagttagacctattttgctgttaaaataggtctggtcttgttataggtcataggaattgtgtagtttgagttgctgcgagaaagagtcaagaacttagacttgaatgggaaggccaactggtgagggaagtggggtattatctactcaaatgttctaagctaatacaaaagaattacctggcaagcatccccctctgcccttcttgaaattctgaatgaacttagaatcagatcaatggtgagacccagttatgtaactttcctttgaagttctaaaggataattgaaaataaagtagtctggcatgtttaacagtaaggagtgacactaaaatttgaatataacagtatgttgcaactatttataatagacctatttatgttttgagaactaagtaaaatgctatgaaaaattcacataaagcacgtaagcaccaaatgaaaatcatttcgtgctttcaaattaatgatactaggcttgtaggatgtcttagaatcatgtttaacaagaagttaaaatatttgcattttgaaatttttatgcagtttggctaaggaggaacttggaaaataacattcatcagcttctacactgcactttttgtatgtggactttgtaaagtttaatgtccgtttaacagttgcttgtttatttgtgtaaacctagctagtaaatattgatgatgttcagggccactgacttgcacagcccttggagcactattcacacagaacacaattcagagtttgttaagacattaacacactttgatatctattccaaaaatacttttgttaccacaggttcattgctgtcatttagacaaaattaatgtactgtgatattttcctactcttaagatgatgttgcccagccgagtactgtcagaaattcagttctgatgtctgtgtgtgcaagatattaatgtggtccctgtaagaaataccagagttggtcatgatttactaaggagatgtaatattttctttgacctgttaggaatataacttgtgtcttgatttgttgcacatcatattttttgcaacattaatgaatttaaataaattaattggaacgtt

SEQ ID NO: 10 - DNA, 1123 bp, homo sapiens
acccgcggcggcctcctcggtgcgcgacccccggctcagaggactctttgctgtcccgcaagatgcggatgctgctggcgctcctggccctctccgcggcgcggccatcggccagtgcagagtcacactggtgctacgaggttcaagccgagtcctccaactacccctgcttggtgccagtcaagtggggtggaaactgccagaaggaccgccagtcccccatcaacatcgtcaccaccaaggcaaaggtggacaaaaaactgggacgcttcttcttctctggctacgataagaagcaaacgtggactgtccaaaataacgggcactcagtgatgatgttgctggagaacaaggccagcatttctggaggaggactgcctgccccataccaggccaaacagttgcacctgcactggtccgacttgccatataagggctcggagcacagcctcgatggggagcactttgccatggagatgcacatagtacatgagaaagagaaggggacatcgaggaatgtgaaagaggcccaggaccctgaagacgaaattgcggtgctggcctttctggtggaggctggaacccaggtgaacgagggcttccagccactggtggaggcactgtctaatatccccaaacctgagatgagcactacgatggcagagagcagcctgttggacctgctccccaaggaggagaaactgaggcactacttccgctacctgggctcactcaccacaccgacctgcgatgagaaggtcgtctggactgtgttccgggagcccattcagcttcacagagaacagatcctggcattctctcagaagctgtactacgacaaggaacagacagtgagcatgaaggacaatgtcaggcccctgcagcagctggggcagcgcacggtgataaagtccggggccccgggtcggccgctgccctgggccctgcctgccctgctgggccccatgctggcctgcctgctggccggcttcctgcgatgatggctcacttctgcacgcagcctctctgttgcctcagctctccaagttccaggcttccggtccttagccttcccaggtgggactttaggcatgattaaaatatggacatatttttggagaaa

SEQ ID NO: 11 - DNA, 3171 bp, homo sapiens
atcttgagactcgctaagcgtcccagccgcatccctcccgcagcgacggcggcccgggacccgcgggctgtgaaccatgaacacccgcaatagagtggtgaactccgggctcggcgcctcccctgcctcccgcccgacccgggatccccaggacccttctgggcggcaaggggagctgagccccgtggaagaccagagagagggtttggaggcagcccctaagggcccttcgcgggagagcgtcgtgcacgcgggccagaggcgcacaagtgcatacaccttgatagcaccaaatataaaccggagaaatgagatacaaagaattgcggagcaggagctggccaacctggagaagtggaaggagcagaacagagctaaaccggttcacctggtgcccagacggctaggtggaagccagtcagaaactgaagtcagacagaaacaacaactccagctgatgcaatctaaatacaagcaaaagctaaaaagagaagaatctgtaagaatcaagaaggaagctgaagaagctgaactccaaaaaatgaaggcaattcagagagagaagagcaataaactggaggagaaaaaaagacttcaagaaaaccttagaagagaagcatttagagagcatcagcaatacaaaaccgctgagttcttgagcaaactgaacacagaatcgccagacagaagtgcctgtcaaagtgctgtttgtggcccacaatcctcaacatggaaacttcctatcctgcctagggatcacagctgggccagaagctgggcttacagagattctctaaaggcagaagaaaacagaaaattgcaaaagatgaaggatgaacaacatcaaaagagtgaattactggaactgaaacggcagcagcaagagcaagaaagagccaaaatccaccagactgaacacaggagggtaaataatgcttttctggaccgactccaaggcaaaagtcaaccaggtggcctcgagcaatctggaggctgttggaatatgaatagcggtaacagctggggttctctattagttttttcgaggcacctaagggtatatgagaaaatattgactcctatctggccttcatcaactgacctcgaaaagcctcatgagatgctttttcttaatgtgattttgttcagcctcactgtttttaccttaatttcaactgcccacacacttgaccgtgcagtcaggagtgactggcttctccttgtcctcatttatgcatgtttggaggagctgattcctgaactcatatttaatctctactgccagggaaatgctacattatttttctaattggaagtataattagagtgatgttggtagggtagaaaaagagggagtcacttgatgctttcaggttaatcagagctatgggtgctacaggcttgtctttctaagtgacatattcttatctaattctcagatcaggttttgaaagctttgggggtctttttagattttaatccctactttctttatggtacaaatatgtacaaaagaaaaaggtcttatattcttttacacaaatttataaataaattttgaactccttctgtataaatgggtcatttttatttttaatgaaaagttattggggttttctctcttgaagggtctcattttaattcccttttccaggccgtatagatcaaatatagtactgtcattactgttggctcttgttttggtcttgacttactaatagtgttaccctgattttcagagggggacagtttatctccagaaaggccaatgtttgtatacacatcagctagacacaaatatagacatcatatgtagtttgtacatgtttcagaaacttgttttttctttgctctgtgtaacctatttcctattgctagttcagttggctttcttattcacttctgtgaccctgaaccagttctcagaccctagagtgtaagagcattgattttctacgctgtgtaatctagctcaatccctctgtcccctccgcctcaccgtcccccagccaccacattgtatagcaaaagcattacattcaatcctagaataaaggtaaatacaacaaatcatctttgcagctggacaactaataatactttgcagcattaagagatcttctgtgttaccagtcactctgttgaaatgaactttccgaatctctttattcaggaaaacatggggttttgaaattcttgggccaagagacataactgaggggttcgcagagctaggcaagggtgcactaggaaagggccacattggtgggtggggggtaacagagaacagatggtgtcaggaagtttctctggagtaaataatgtggatattcttggtttccctctcctccgccagctgaagctgtgttagtgctgttgacactaatataaaatgtttggtccatttgaaatccttgtcattgccttatatgggggaaactcaatcccccagcctgtgttggaaatatcaccaaactgattgtaaatgtgcggctgtagcagacattttagtgtggtggtgtgcagccatttcggccctacacctgccagcctggctaccttacagttgtgttccgatttttgcgtctatgcttggtgtgcctcacttgctgcattttccagcatgcaaccaggagttgacgtaggaaaaagggatgctttcttactttggaagctctcagggaagttggtgtcaatttctcctccactgcctggcctaccctgcactcccaaagattttgtgcagatgggtagttccattttttaaaaattgtgcagatatggaaaattgtgacttacttcatgaccagaactatctagaatatgtgtgggggtataaacatcttgcttaaccaaatatctatgtaggcagaggtaaccaggagagaagcaagacttgctgcctaaaggagcccaccattttacttttcacatttaatctgccacgttgaatcaattggaataaaacctgactcgcaggtgactggacaggaaatcccaaagttccaccatttctatgcttaattttaatgtccccccgcttttttttttgtagaaaataaaaacaagaaaatcgttccaatgtaagatgtttgttatagaaactttaggcaatacaggtgtgtaataaaatgtttaataaacttctaaacacttttgtatttggattta

SEQ ID NO: 12 - DNA, 5996 bp, homo sapiens
tccccgccgtgcctcctcgctggccgcgctccctcccggtgccggcttctctgagtcaccaacctgaggctgccccggccgcctgcgcacccggcagcaccatgacagatcaggcctttgtgacactaaccacaaacgatgcctacgccaaaggtgccctggtcctgggatcatctctgaaacagcacaggaccaccaggaggctggtcgtgctcgccacccctcaggtctcagactccatgagaaaagttttagagacagtctttgatgaagtcatcatggtagatgtcttggacagtggcgattctgctcatctaaccttaatgaagaggccagagttgggtgtcacgctgacaaagctccactgctggtcgcttacacagtattcaaaatgtgtattcatggatgcagatactctggtcctagcaaatattgatgatctttttgacagagaagaattgtcagcagcaccagacccagggtggcctgactgcttcaattccggagtcttcgtttatcagccttcagttgaaacatacaatcagctgttgcatcttgcttctgagcaaggtagttttgatggtggggaccaaggcatactgaacacattttttagcagctgggcaacaacagatatcagaaaacacctgccgtttatttataacctaagcagcatctctatatactcctacctcccggcatttaaagtgtttggtgcaagtgccaaagttgtgcatttcctgggacgagtcaaaccatggaattatacttatgatcccaaaacaaaaagtgtcaaaagtgaggcccatgatcccaacatgactcatccagagtttctcatcctgtggtggaacatctttaccaccaacgttttacctctgcttcaacaatttggccttgtcaaagacacctgctcatatgtaaatgtgctttcagacttggtctatacactggctttctcttgtggcttctgtagaaaggaagatgtctcaggagccatatcacatctgtcccttggggagatcccagctatggcacagccgtttgtatcctcggaagaacggaaggaacgatgggaacagggccaggctgattatatgggagcagattcctttgacaacatcaagaggaaacttgacacttacctccagtagaaacactgcatttttctgtgaacacatccacttcacaagccttgtttctgatacttagtatctagagctgggttgagaaaagtctgttacagttgctagaggttttcattaaaacttatcagatgagaggcttttttaggataagaggtgagaactgggcaaaagttgtgaagcagcaattctgttatatggacagtgttctgctttttaatcctatttagcttgtttcagaaattctcacttttgttgactgccaacatacaaagtaagggaaactcaagatattaagatggctgtatcagttcttaaaatctgcagagcctggttcaaaatcagtcactcccttcagaagcagacatggcatctgttccttgcttgcttgttggttgtgtacctttcacgagacctgaattttagaattgcccagtgctgccagagtgagtgagtgtaattctcctttcaggtaaagataggctatctcaacactgctgagtgattcataaacatatcaaccaatagcattaacccattttatttcctgtccttagtgtctgaagatgctcaccagttttctgtgtacagtaaggcagcatgctaaaatgcttttgttcagttctgtatatttgaaaatagcagtgtgttctctgatggttacctgcagtggcaccctgtacaaaaaataaaagacttattgctgtatcttggttgtttaattaaattaaggaatttcaccatacacccttgaacaaatctattagggaatttttcacaatttttggaatttgtcatagttttaaaaaagtgtaaagcttgacattgggatatatgctttaaaaactggtatctatgatttcaatctaattgtttttctgtgatggtgatggatctgacagatcagaacaaaccgagatcaaacttacatagtgtcatccacactgcacctctctattaagtgggtatttagctatttagaatattttaaccttaaagtcattcctaactgtcagaatcaatactgggtccagtccctactagtattttatctatgatttaaaatgatagaatggaaaagcgtatttattaaatttatagatcattctaggttgtaagagatggcaagctaaagaagggtaggatccaagatgaccttttaaaactaaaatgagtggtcagaaatgattctatggaagaaaacaggacagttaaccatgacaggaagaaactttagaggtttgattgggcaaaagctgcaaagcttttgtgacttagccttcaaagtcaccctccttattcatattgatgtgaattaatatacaagcatatagacagcttaagtcaagaatggttggggccaccttgaaggctgttaccataggagctcaataagaacctgaggatttacctagaattataaaatattaaatgataaatgacttctgaaactagctatttggactggtgaagaatgagtcgctattgatcttcaagtacaatgaagcatttaccaaagatttatttaagtgcctccatgtgtcagatgctgtggtacaaagaaggacttctcaaaattttagctagtcagaggtctttctggcttccgagtccctggttaagatgaacagaaacacagtcttcagatataaaatgtcttatttttgtggccattcagttgcattcaacgttaattttttctatttactaccgttcattctctattttttacacagtagcataacaaagctctaaggtggaaaagctgacatagttttaaatttttttttttttttttttttttcttgaggcagagtcttgctctgtcagtcagtcaccaggctggagtgcagtggtgcagtctcggctcactgcaacctctgcctcctggattcaagagattctcctacctcagcctcctgagtagctgggactacaggcacgcaccaccacgcccagctaaattttgtatttttagtagagacaggtttcaccatgttgccaggatggtctcaatctcttgacctcgtgatccaccggccttggcctcccaaagtgctgggattacaggcgtgagccactgcacccagcaaatttttaaatttcaaataagtagtgaaggctattattaacttttggaatcagaaagaatgacaagcttaccataagacatagcatataatgctgtcaagttatttggctagaaaatcactgaactaaacattcttttccttctatgatctatgtcttaaggtgaagtattaactaacttttccatgtaaagctatacaaatattggaaaatcttttctagggagtccagaatactaagggttacttagtaaaatgtataaaaaggcaacagtaattcaaattacaagatttatatttgcagaggtgatccatatatacttatccccttgcagtggctggtatgacctttggttgtaagacaaacttgcccacaacagaggtcaaatccatgcctttggagattagctccatggtggatggagctatggtttatgcataaagtaaatgtttgtttaccttaattctccttatacccatattgtcctgctgtataacacattttgcagatattttgaagttaatgtgttaaaaacttgaggttaaacatttgagtttttgttaagagccaaacatcaaatgtgcccttatatttttaatgaatctcatccaaatgctaatgcataaaccttgacaagtagtataaataaaacaagaaaaaaatacagcaatgtctttgccattccccaaaacaaagcacacactgccgaagatcattagtactcactggtaacaaactacatagggttagtttgtatttccaattctagagctgtaattttaaggacaaaatgtacaatgattgattaagagtgctatctgtgtatatataggtattatcacaactcctttttttcttccagatgaagaaattaattgggaccaatgtttttagatcaaggcattttaaataagcactcttgatttctgaacaagaatttcaaccagctaaattgagcaaaataaagttagttaggatatgaggacattattctgttacagtaatcttcatgtactctcaaaaaaatgtaacacttgcatagaaatgtcacaattaatgaaggattttatttgaagataaagtcaaaattatggcaccgaggaaggtaataaacatttgaaatttttattgatttttaaatttaaaatccagttttaaccacaaaattgtttgaatcacaagtggtaatacaatgtcttcaatatttttctaaagttatttttctatataataataagacaacagcatagcatataggaagttttcattccagtggcttttttatatatttatccttcttaggaaggacaaattaaattttttaaattaaacttttaaaatataacaacatctaacagaactgtacaaaacaaagagacattttttaaacaacttgccaaacttacttatgagtgtgttttaaaaacaactttgtaaatgtctgggcaaagaagcaagctgtcctccctttaccttcatagtgagtttgtaaggctttgtctttgtaagcagaaagagtagactgtgttgttttttgccaaaaactgtttatacttaatctcactgaagtattgctatatggagaacccatactctgatcaacttgattttttgtgtgtaatgcttgatctaccaggtaacttcccaactgctcctaatgctagcgggctaatcccacattattattccactatcatccctgcagaaaggtcttggttttgatgaaaatcagccctttccttacctgctactgcctcaaaaagggaccaggaagattctagctggctaattcactgttccctttgagcaagaaaacggcacagggagaaaaggacttatctggtgagagatttggcatataccttcaatgtgtgccctataacacaacattgtctccgatctcatctttctatcaaatgacttccaacactcttaagtctcaggtattcttaaatctgtatcatcaaacatgaagcttctcttgtttgttagagtaattaatctttctttggattaaagtttccctttgaaataaaaccacctacctaatctgactgctaaatttctagcttctttgttttaaatatgctcaggagtcaacccaaattctgcagcaaataagtttgcttattaacaaaaaagtaaaaaaaaaagaaaagaaaaaagatgactaattctacagatagctgtaaggatgaattactcaagttcaaaatcaaattctgattctaaacacataacaattgtttacattcaggattaagatgtctttaagagttgaaacgactttggagatcatccagcccaacttccatccagatatcacgcctctcacatatagtagtcttctgaattataaaaatttataaagttacttccaaaaaaagctacataaataaaaattatctatttatagaaatatctatttagcagttccataatttaaaatattcaaatcaaattgggtaggactggtttgcctctcactcccacagactatatttatacctcagacacagcaagttacatttaaacaatgagtgtagtactacttaactaaaatggaaaaaatagtactcttaacataatccctaattttttcatgaacataaaactccaagtcatttatgtgaactatatctcaatgtagctgtaggaaaaataaaaacctgtgctaacctggactttggtctcatttaagatttggttctggaatgcaaatatggtttttgaaagcccaataaaattaattcttgtata

SEQ ID NO: 13 - DNA, 3511 bp, homo sapiens
agtagctgaggctgcggttccccgacgccacgcagctgcgcgcagctggttcccgctctgcagcgcaacgcctgaggcagtgggcgcgctcagtcccgggaccaggcgttctctcctctcgcctctgggcctgggaccccgcaaagcggcgatggagcggaggtcgcggaggaagtcgcggcgcaacgggcgctcgaccgcgggcaaggccgccgcgacccagcccgcgaagtctccgggcgcacagctctggctctttcccagcgccgcgggcctccaccgcgcgctgctccggagggtggaggtgacgcgccaactctgctgctcgccggggcgcctcgcggtcttggaacgcggcggggcgggcgtccaggttcaccagctgctcgccgggagcggcggcgcccggacgccgaaatgcattaaattaggaaaaaacatgaagatacattccgtggaccaaggagcagagcacatgctgattctctcatcagatggaaaaccatttgagtatgacaactatagcatgaaacatctaaggtttgaaagcattttacaagaaaaaaaaataattcagatcacatgtggagattaccattctcttgcactctcaaaaggtggtgagctttttgcctggggacagaacctgcatgggcagcttggagttggaaggaaatttccctcaaccaccacaccacagattgtggagcacctcgcaggagtacccttggctcagatttctgccggagaagcccacagcatggccttatccatgtctggcaacatttattcatggggaaaaaatgaatgtggacaactaggcctgggccacactgagagtaaagatgatccatcccttattgaaggactagacaatcagaaagttgaatttgtcgcttgtggtggctctcacagtgccctactcacacaggatgggctgctgtttactttcggtgctggaaaacatgggcaacttggtcataattcaacacagaatgagctaagaccctgtttggtggctgagcttgttgggtatagagtgactcagatagcatgtggaaggtggcacacacttgcctatgtttctgatttgggaaaggtcttttcctttggttctggaaaagatggacaactgggaaatggtggaacacgtgaccagctgatgccgcttccagtgaaagtatcatcaagtgaagaactcaaacttgaaagccatacctcagaaaaggagttaataatgattgctggagggaatcaaagcattttgctctggataaagaaagagaattcatatgttaatctgaagaggacaattcctactctgaatgaagggactgtaaagagatggattgctgatgtggagactaaacggtggcagagcacaaaaagggaaatccaagagatattttcatctcctgcttgtctaactggaagttttttaaggaaaagaagaactacagaaatgatgcctgtttatttggacttaaataaagcaagaaacatcttcaaggagttaacccaaaaggactggattactaacatgataaccacctgcctcaaagataatctgctcaaaagacttccatttcattctccaccccaagaagctttagaaattttcttccttctcccagaatgtcctatgatgcatatttccaacaactgggagagccttgtggttccatttgcaaaggttgtttgtaaaatgagtgaccagtcttcactggttctggaagagtattgggcaactctgcaagaatccactttcagcaaactggtccagatgtttaaaacagccgtcatatgccagttggattactgggatgaaagtgctgaggagaatggtaatgttcaagctctcctagaaatgttgaagaagctgcacagggtaaaccaggtgaaatgtcaactacctgaaagtattttccaagtagacgaactcttgcaccgtctcaatttttttgtagaagtatgcagaaggtacttgtggaaaatgactgtggacgcttcagaaaatgtacaatgctgcgtcatattcagtcactttccatttatctttaataatctgtcgaaaattaaactactacatacagacacacttttaaaaatagagagtaaaaaacataaagcttatcttaggtcggcagcaattgaggaagaaagagagtctgaattcgctttgaggcccacgtttgatctaacagtcagaaggaatcacttgattgaggatgttttgaatcagctaagtcaatttgagaatgaagacctgaggaaagagttatgggtttcatttagtggagaaattgggtatgacctcggaggagtcaagaaagagttcttctactgtctgtttgcagagatgatccagccggaatatgggatgttcatgtatcctgaaggggcttcctgcatgtggtttcctgtcaagcctaaatttgagaagaaaagatacttcttttttggggttctatgtggactttccctgttcaattgcaatgttgccaaccttcctttcccactggcactgtttaagaaacttttggaccaaatgccatcattggaagacttgaaagaactcagtcctgatttgggaaagaatttgcaaacacttctggatgatgaaggtgataactttgaggaagtattttacatccattttaatgtgcactgggacagaaacgacacaaacttaattcctaatggaagtagcataactgtcaaccagactaacaagagagactatgtttctaagtatatcaattacattttcaacgactctgtaaaggcggtttatgaagaatttcggagaggattttataaaatgtgcgacgaagacattatcaaattattccaccccgaagaactgaaggatgtgattgttggaaatacagattatgattggaaaacatttgaaaagaatgcacgttatgaaccaggatataacagttcacatcccaccatagtgatgttttggaaggctttccacaaattgactctggaagaaaagaaaaaattccttgtatttcttacaggaactgacagactacaaatgaaagatttaaataatatgaaaataacattttgctgtcctgaaagttggaatgaaagagaccctataagagcactgacatgtttcagtgtcctcttcctccctaaatattctacaatggaaacagttgaagaagcgcttcaagaagccatcaacaacaacagaggatttggctgaccagcttgcttgtccaacagccttattttgttgttgttatcgttgttgttgttgttgttgttgttgtttctctactttgttttgttttaggcttttagcagcctgaagccatggtttttcatttctgtctctagtgataagcaggaaagagggatgaagaagagggtttactggccggttagaacccgtgactgtattctctcccttggatacccctatgcctacatcatattccttacctcttttgggaaatatttttcaaaaataaaataaccgaaaaattaa

SEQ ID NO: 14 - DNA, 1534 bp, homo sapiens
actggaaaagatagtgaccttaccagggccaaagtttgtagacacaggaattacgaaatggagaagggggagaagtgagctagtggcagcataaaaagaccagcagatgccccacagcactgctcttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgaccca

SEQ ID NO: 15 - DNA, 2619 bp, homo sapiens
gagctcgcccaccgcccgccccagcagtggctgcaccatgcacgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctggatgagcagtttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcaggactcatgcccgttgcacagcagccggtttattgtgcttcaaagcatggcatagttggattcacacgctcagcagcgttggctgctaatcttatgaacagtggtgtgagactgaatgccatttgtccaggctttgttaacacagccatccttgaatcaattgaaaaagaagaaaacatgggacaatatatagaatataaggatcatatcaaggatatgattaaatactatggaattttggacccaccattgattgccaatggattgataacactcattgaagatgatgctttaaatggtgctattatgaagatcacaacttctaagggaattcattttcaagactatgatacaactccatttcaagcaaaaacccaatgaacagcttatgtgttagccatagctgaaaataagcacaaatagcttatattcagatcctatcttcatttgaatatagcttttaaatgaaatgttacagtttgaagttttccttcatgcacttggtgataaacgttttctaaatttttagttaagtatatggataaaaagttatgaactattaaaaatgtgatgtggaccaaaggctaggttgtaatcttgatagtctaaaaaatgatcaaaacaaatgattttcaaggaatattcaatattctgcctttcagaaagtgtatttatatctgtgcttcataaatattaatgttcttcagaacatcattttaaaggagatacttgaattgttatttaaatcaaaccagatgtaaaacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcagcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtccaaatttcaaaagtgcctacccttcaataaaacttttttattctgatctccataaattacttagtcttctatgtatagctatcaaggaaataaaaccaattttgccacagccacaactgtaaatgtttttgtacccatgctgaaactcataacaacacagacataaaaatagctgtgaggttttgctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttttcctctgtcgtgtaaatagtataaccttttcatttatggacaattttttggactagtagccttcaatatacattctgctttgaattaattttttcaaatcaataaattatgtagacatttaaaatcaaatatcaagtagaattgaaaaatgtgagttacataagttaaaaacttactttaaatcttaccttctataggtagctctaaataaattcatatggttatatggcatctctggtgtatactgattgagaaaataattaaactgaagttaggggaggggga

SEQ ID NO: 16 - DNA, 652 bp, homo sapiens
aacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaa

SEQ ID NO: 17 - DNA, 1682 bp, homo sapiens
cattttgtgcctgcctagctatccagacagagcagctaccctcagctctagctgatactacagacagtacaacagatcaagaagtatggcagtgacaactcgtttgacatggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcagaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaagattcactggatgaaagaaagataaaaggggtcattgagctcaggaagagcttactgtctgccttgagaacttatgaaccatatggatccctggttcaacaaatacgaattctgctgctgggtccaattggagctgggaagtccagctttttcaactcagtgaggtctgttttccaagggcatgtaacgcatcaggctttggtgggcactaatacaactgggatatctgagaagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcgaagggagttggtaaacgctggtgtggtacatgtggctttgctcactcatgtggatagcatggatttgattacaaaaggtgaccttatagaaatagagagatgtgagcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatagatatgtgaaaggttcacgtaaatttcctcacatcacagaagattaaaattcagaaaggagaaaacacagaccaaagagaagtatctaagaccaaagggatgtgttttattaatgtctaggatgaagaaatgcatagaacattgtagtacttgtaaataactagaaataacatgatttagtcataattgtgaaaaataataataatttttcttggatttatgttctgtatctgtgaaaaaataaatttcttataaaactcggg

SEQ ID NO: 18 - DNA, 2390 bp, homo sapiens
gcagacaggaagacttctgaagaacaaatcagcctggtcaccagcttttcggaacagcagagacacagagggcagtcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgtta

SEQ ID NO: 19 - DNA, 4029 bp, homo sapiens
agtttctgagcgctcggcatctgattcaatctccagtttcctgttcttgctggggctggggtctctcctttaacaaagacacgccgcgcggccgagtccaggggctgcagaggcctggcgcgcgcacgcgcacgcgcacgcccaccgcgcggcttcccgcggtccccggtgctgaggagagagcgatccgagggactgcgccgcccggacggcctgcagagcgctgccatcatgagtgaaattcgtaaggacaccttgaaggccattctgttggagttagaatgtcattttacatggaatttacttaaggaagacattgatctgtttgaggtagaagatacaattgggcaacagcttgaatttcttaccacaaaatctagacttgctctttataacctattggcctatgtgaaacacctaaaaggccaaaataaagacgcccttgagtgcttggaacaagcagaagaaataatccagcaagaacactcagacaaagaagaagtacgaagcctggtcacttggggaaactatgcctgggtgtattatcacatggaccagcttgaagaagctcagaagtatacaggtaagatagggaatgtctgtaagaaattgtccagtccttctaactacaagttggagtgtcctgagactgactgtgagaaaggctgggcactcttgaaatttggaggaaagtattatcaaaaggctaaagcggcttttgagaaggctctggaagtggagcctgacaatccagaatttaacatcggctatgctatcacagtgtatcggctggatgattctgatagagaagggtctgtaaagagcttttctctggggcctttgagaaaggctgttaccctgaacccagataacagctatattaaggtttttctggcactgaagcttcaagatgtacatgcagaagctgaaggggaaaagtatattgaagaaatcctggaccaaatatcatcccagccttacgtccttcgttatgcagccaagttctataggagaaaaaattcctggaacaaagctctcgaacttttaaaaaaggccttggaggtgacaccaacttcttctttcctgcatcaccagatgggactttgctacagggcacaaatgatccaaatcaagaaggccacacacaacagacctaaaggaaaggataaactaaaggttgatgagctgatttcatctgctatatttcatttcaaagcagccatggaacgagactctatgtttgcatttgcctacacagacctggccaacatgtacgctgaaggaggccagtatagcaatgctgaggacattttccggaaagctcttcgtctggagaacataaccgatgatcacaaacatcagatccattaccactatggccgctttcaggaatttcaccgtaaatcagaaaatactgccatccatcattatttagaagccttaaaggtcaaagacagatcaccccttcgcaccaaactgacaagtgctctgaagaaattgtctaccaagagactttgtcacaatgctttagatgtgcagagtttaagtgccctagggtttgtttacaagctggaaggagaaaagaggcaagctgctgagtactatgagaaggcacaaaagatagatccagaaaatgcagaattcctgactgctctctgtgagctccgactttccatttaaatacatactctaggaaattagctctaagtttttcccttcattttgggttctcctgtttgttttttttttattattttaatcccttgtttattatagagctaatatttattgaatagttattgtgtaccaagcattgtgctaaatactttatatgcattatgatgaatcttgtgcggttttctttctttttttctttttaattaaaatactataatccattgagaaatagcaatattctagctattgtaacttctaaaaatggtatggccattagatctgtgctttttatctctgctctttgaatttctcatattatatagtaaatatattcctacgtaaacctttgatacctagatcaggaatactcttccaggagtacaaaattacattattgatagttaagctcttaattgtgtagcttgcaaaagacagcactttttagttacagatgttttgactttgatgaggatatttagctatcaatctaatagtcacctaaaatatcttttttgttggaaaaaagtttataataaaaaagtttgtcatctctagtgacttcaataaagaaaaaactagaagaggagaaaaaggatttcctcaaattttaaatatgtaacttcagggattcaatccccaaatgtttattaagtagctagaaataattatgtggaaaaaaatgaataatggaaaatagtgagtctcaaattgttctctttttttttttaactaaaacaaatctgcaatgaatctagatgcaattaattttattccttccaactaaaattacaatatttttaggttaaaattattgagatataaagcagccattgggaaattgggagaaatgataaacaaatggaaaaagaagatgtccctaacctacacccatagattaccaaggtttcagtgtactagttttgaatctgttctgaatggagtttttataccctcaatttctggcctttggctattttagcatttcaaagtgacttctatgaagctttttttttaatgtgaaattttcagaatgttgtttttttcatgtagatactccaggaagagttaagcactgctttcagttttaatatccaccttgaggggtcgctgcttgagggctcttatcccaggggactttttaattcggatgttacttaatgtggcttctctaatgtagtttctttgattaccgactacacaattatgtaccatcacagtattagtggaaaagtaccatgtgatttaattctccattcctccaatgtaactcttaaaattattatgtatgtgtgtgtgttttactttttgttttttatcatctttaaaatttctattatggtttgattattataaaaataatgaattctcactgtaaatttcaaaaaaaaattacaaaagtatgtgaatttaaaaatgagagcagtcctctcaccctaccacagttccacaccctcaaggtaaacttataacttataatttgatatgtaaacttccagatcttttttctatgcgtaatcagacatacatatatactgcagtgtatctcacgtattaatttttaaaaatcttttgttttacttaattctgtttttattattattattattttgtttgatctattaaggaagaacaaggaagggaatgatctttactcaagaatttcagaaagtcagcactgaagtcctgacctatcagtagacacatttgtccctttcagatattttaggatattctagcaaagcaggccatttctcccacctgaaagtacataacttctatcacttgccacataattaaaagaactcacattaagcggttactcagacagttaatcatagaaaagattatttgcttcatcagttcatagaaaagattatttgcttcatcagttaacttgtttttataaatcagggctgtgttcatacacagaaggggcctgagatttctgcactttaaacaagctcctcctaggtgaggatgctgtggctgttctaattacattttgagtagtaaggtctacagcattgttcctcaaacttggctacgtattggaatcacctaaaaagttaaaacaaaacatggatgtctgggtcccgccccatagagaatgacttaattggcatggggtgcagtccaggcatcatgatttttagatttcccagttggaacttgtgcagcaaagtttgggagctactgatggacatgtgaaaagtaagtataaatggaataaaattaattaggctaataggcttaacccaggaaatcctaagttccttgaatatccagtttgcatttggactcctcatcatatacttggtatataatactctaataaaagctgcctgagttgaa

SEQ ID NO: 20 - DNA, 3471 bp, homo sapiens
tcaggaggcggagatcgctgcttctcacctactttctgaacttggcctccgcagtcgcgacctggcgtgaaggaggagctgccgcccccgccccagcctcggggacgcctctctgaagagaagccatttgaagcagaatccaaaccatgaattgtagagaattacccttgaccctttgggtgcttatatctgtaagcactgcagaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagataggaccacctatttgcaggaaaacaagctcagggctccactgattctacattatgaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttaatcttcagaaacagtgaacgccaaaaagaactcaagatattctggggactgagcatatgaacctgttcataacaaaggctgtgactcgaaataattaactttgtcaaaatcctgctcacaatttgaagatgaaacttgtcattaggttggcgggaatgagactaaagattgcgctgtgggctgtggtcacgtgctcccagaagacctggaattcaaaagaaatggagctattctttttctccctctttcataactggatgcagctgctcatactcaatcccatattcagcaagtgtgaagctggacgtgatgcaaaataaccgatgccctacaaaaagggcgcatctttaagagttttaatgccagtgcttaattcgaatgaggggattttaagtgtctgaagaggcattttctagggaccagtgggtgactgagtaactgaaatgctgctttcactccctaacaccatggatctggttgtgcataggatgtgggaggaggggctggcagggccgccttcagaggctgcagggcctcagcctcaggatgcatttaatgtatcctggccacagttgcagccaacggttcttgaaagctcggtaaggccctgcaacgcagagcctgcttatgtggatctatttatgggaacttcttaaaaggaccccagaatagctctttatctttcacaagagacacaaattctaattgagttaattatctgggcctttcactttggatgctctgaaacatttgttgattttgtgtgaatgtttatatcaaaatgtttgccaggttgtattagccattgaatagcaaaaaactgatagttacttgcttgttttttaaaaattacatattaaaaatgcccttggcataaggcagcatggtgtggcagttaagagatgggctgtgcagcccatcctgagctccagtcctgagtttgctacttacttctgtggcctctggaaccttatccaacctcttggtgcttcagtttcctcatctgtgaaattagaatttataataattgcacctacctcccaggggtaactaaatgaataaatataataaagtacttacagtggttcctgacacagactcagcactccgtcagtgttgccatgactatttttattatcattattaatgattacttagatcaattatttagcagtggactaatggaagctacagagcagggaagggaagcagatctagggaggaaggcagttttgatttgaggaggtttgcacatgtagagaagcatactggagaagcatatccagagggcgaaagatatctctccattgtgcatctgcctcttttgacgttggaagacacatgtcttaccccccaaagggagcccagcactgggagccttcttgatgatctcaaaaataatagctattcaagaaaatcaccaagtgactgtgaaaccgtcagttcggaaggctggttagaacatgtgggagcaacatgaatgttctacaaaagtttaaagcagagattgtttcaaatgggtgtagtagatattactgaaaaccaaaaaagagtgagattgtcagtgtaagaatgtgatttaatgtttgtagtgcttacaattttgtgtaccaactggatgactaaaaagagtaaaataatttaattaatagctcatattttatgtgtgaaaacatgttagtgaacatatataatcaaaatagatttcattgctattgcatagtctctaatacatagaatgattttgcttttctcttttattatacttgctttaaaatacttgaaatatattttgcattaaatgcatttcaagttaaatgtcttaaatgtatacattagatgtgtgttttaaaatgcataaaacacgttgaaatacattaatgaaccatt

SEQ ID NO: 21 - DNA, 1449 bp, homo sapiens
tgcccttcccgccagcgcaggtgtgggacgggcggcggactaggcacagagctgcgggagcaggcacagggagtgtggagcctggcggcgggacggcgggatccggtgggagccggagtcccgccgaggggggctggaggtggaggggcccggcgaggccgcgatgaagccgagcggcgaggaccaggcggcgctggcggccggcccctgggaggagtgcttccaggcggccgtgcagctggcgctgcgggcaggacagatcatcagaaaagcccttactgaggaaaaacgtgtctcaacaaaaacatcagctgcagatcttgtgacagaaacagatcaccttgtggaagatttaattatttctgagttgcgagagaggtttccttcacacaggttcattgcagaagaggccgcggcttctggggccaagtgtgtgctcacccacagcccgacgtggatcatcgaccccatcgacggcacctgcaattttgtgcacagattcccgactgtggcggttagcattggatttgctgttcgacaagagcttgaattcggagtgatttaccactgcacagaggagcggctgtacacgggccggcggggtcggggcgccttctgcaatggccagcggctccgggtctccggggagacagatctctcaaaggccttggttctgacagaaattggccccaaacgtgaccctgcgaccctgaagctgttcctgagtaacatggagcggctgctgcatgccaaggcgcatggggtccgagtgattggaagctccacattggcactctgccacctggcctcaggggccgcggatgcctattaccagtttggcctgcactgctgggatctggcggctgccacagtcatcatcagagaagcaggcggcatcgtgatagacacttcgggtggacccctcgacctcatggcttgcagagtggttgcggccagcacccgggagatggcgatgctcatagctcaggccttacagacgattaactatgggcgggatgatgagaagtgactgcggctgaggcaaagctgctcccaaggcctccctgggctgctgtgggctcctggggaggtggccctcgtggcccacgctccatgccagtggctcacgctctgctcctggctaccccagagggagttgtcacgctacagtgagtggctggccttttaaatcgacgtctctctcaccaggatttggtgtttagctgtttctctctttaatctcacgtagcctttttcaggttagtacgtgttcttctgtcagggccaaaactcaaatctcctgtgaaatacgtattgataatccaatcttgatttttccccccagaatataaatctcaggtaataaggctttagaactgctgataaagcggatcgttctcaggccctccccccggagtacttcagaatgcaataaatcaaaataatggc

SEQ ID NO: 22 - DNA, 637 bp, homo sapiens
ggcggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgagcatcctggtgaggaataacaagggccgcagcagcacctacgaggtacggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaaagagaaa

SEQ ID NO: 23 - DNA, 820 bp, homo sapiens
actcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcagg

SEQ ID NO: 24 - DNA, 1126 bp, homo sapiens
agagcgcgcgaggttcggggagctcggccaggctgctggtacctgcgtccgcccggcggacaggctgctttggtttgtgacctccaggcaggacggccatcctctccagaatgaagatcttcttgccagtgctgctggctgcccttctgggtgtggagcgagccagctcgctgatgtgcttctcctgcttgaaccagaagagcaatctgtactgcctgaagccgaccatctgctccgaccaggacaactactgcgtgactgtgtctgctagtgccggcattgggaatctcgtgacatttggccacagcctgagcaagacctgttccccggcctgccccatcccagaaggcgtcaatgttggtgtggcttccatgggcatcagctgctgccagagctttctgtgcaatttcagtgcggccgatggcgggctgcgggcaagcgtcaccctgctgggtgccgggctgctgctgagcctgctgccggccctgctgcggtttggcccctgaccgcccagaccctgtcccccgatcccccagctcaggaaggaaagcccagccctttctggatcccacagtgtatgggagcccctgactcctcacgtgcctgatctgtgcccttggtcccaggtcaggcccaccccctgcacctccacctgccccagcccctgcctctgccccaagtggggccagctgccctcacttctggggtggatgatgtgaccttccttgggggaccgcggaagggacgagggttccctggagtcttacggtccaacatcaggaccaagtcccatggacatgctgacagggtccccagggagaccgtgtcagtagggatgtgtgcctggctgtgtacgtgggtgtgcagtgcacgtgagagcacgtggcggcttctgggggccatgtttggggagggaggtgtgccagcagcctggagagcctcagtccctgtagccccctgccctggcacagctgcatgcacttcaagggcagcctttgggggttggggtttctgccacttccgggtctaggccctgccccaaatccagccagtcctgccccagcccacccccacattggagccctcctgctgctttggtgcctcaaataaatacagatgtccccca

SEQ ID NO: 25 - DNA, 3055 bp, homo sapiens
gacacatgatgctgtgaacgtcagggtgctcgccagggaagggccctacccagagggacagaaagaaagccaggaggggtagagtttgaagagaagatcatgttctccctgaagacgcttccatttctgctcttactccatgtgcagatttccaaggcctttcctgtatcttctaaagagaaaaatacaaaaactgttcaggactacctggaaaagttctaccaattaccaagcaaccagtatcagtctacaaggaagaatggcactaatgtgatcgttgaaaagcttaaagaaatgcagcgattttttgggttgaatgtgacggggaagccaaatgaggaaactctggacatgatgaaaaagcctcgctgtggagtgcctgacagtggtggttttatgttaaccccaggaaaccccaagtgggaacgcactaacttgacctacaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagagatcacggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggactttcaagcaaccctatccaacctactggaccaagcacacccaaaccctgtgaccccagtttgacatttgatgctatcaccacactccgtggagaaatacttttctttaaagacaggtacttctggagaaggcatcctcagctacaaagagtcgaaatgaattttatttctctattctggccatcccttccaactggtatacaggctgcttatgaagattttgacagagacctcattttcctatttaaaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggagatatgataaccaaagacaattcatggagccaggttatcccaaaagcatatcaggtgcctttccaggaatagagagtaaagttgatgcagttttccagcaagaacatttcttccatgtcttcagtggaccaagatattacgcatttgatcttattgctcagagagttaccagagttgcaagaggcaataaatggcttaactgtagatatggctgaagcaaaatcaaatgtggctgtatccactttcagaatgttgaagggaagttcagcaagcattttcgttacattgtgtcctgcttatacttttctcaatattaagtcattgtttcccatcactgtatccattctacctgtcctccgtgaaaatatgtttggaatattccactatttgcagaggcttattcagttcttacacattccatcttacattagtgattccatcaaagagaaggaaagtaagcctttttgtcacctcaatatttactatttcaatacttacatatctgacttctaggatttattgttatattacttgcctatctgacttcatacatccctcagtttcttaaaatgtcctatgtatatcttctacatgcaatttagaactagattttggttagaagtaaggattataaacaacctagacagtacccttggcctttacagaaaatatggtgctgttttctacccttggaaagaaatgtagatgatatgtttcgtgggttgaattgtgtcccccataaaagatatgttgaagttctaaccccaggtacccatgaatgtgagcttaccagggtctttgcagatgtaattagttaagttaaggtgagatcacactgaattagggtgggctctaaatccattatgactgttgttcttataagaagaagagaggcatagtcacctaggggaggaggccgtatgaagacagaggcagagattggagtgacgcatctccaagccaaggaattccaaggactgtaagccaccagtagaagctttgaagaggcaaggaaggattccctccaatagccttcaagtgtgaccctgctgacacctgcagaattcggacttctatcctccaaaaccgtgagggaataaatttcctttgttttaagccaccaactttgcaatactttgttacagcaaccctagacatgaggtactagacacagtacatctacacatatgaaaatgaatcaacacagaatgcagaagtagaacccttgctaaggactactgggcatcttcccaggacagcagccaaaagagaaccaccacttcctctcctgcctcctccttgctctctcctagagtccaaacccaaatgggccagttggatctgatgttcgtcagttctttacttctatttcctggggtactcaggagggcacacactatagataacttgggttagctgcataaaattcaatgtctcattaagttgcattaaactgagcttagatgtgtaagtttgctaacggatgggtttttttgttaagaactataggatttatgggaccaagtctagcgagtccagatatcaaaatcattataatgttatatttgctgttattagaatataatatagcttattatacaataaatatgtagactgtaaaatatatttctcactagtacctcctattttctttctctgttgaagtttttaaatcccacagataattaaattggcacctttatgcttgttcaaaaattaaaataatctattaaataagttcaaattaaagatttttacttcaaatga

SEQ ID NO: 26 - DNA, 1522 bp, homo sapiens
agagccctgcatgcatcagacctacagcagacattgcaggcctgaagaaagcaccttttctgctgccatgacaaccatgcaaggaatggaacaggccatgccaggggctggccctggtgtgccccagctgggaaacatggctgtcatacattcacatctgtggaaaggattgcaagagaagttcttgaagggagaacccaaagtccttggggttgtgcagattctgactgccctgatgagccttagcatgggaataacaatgatgtgtatggcatctaatacttatggaagtaaccctatttccgtgtatatcgggtacacaatttgggggtcagtaatgtttattatttcaggatccttgtcaattgcagcaggaattagaactacaaaaggcctggtccgaggtagtctaggaatgaatatcaccagctctgtactggctgcatcagggatcttaatcaacacatttagcttggcgttttattcattccatcacccttactgtaactactatggcaactcaaataattgtcatgggactatgtccatcttaatgggtctggatggcatggtgctcctcctaagtgtgctggaattctgcattgctgtgtccctctctgcctttggatgtaaagtgctctgttgtacccctggtggggttgtgttaattctgccatcacattctcacatggcagaaacagcatctcccacaccacttaatgaggtttgaggccaccaaaagatcaacagacaaatgctccagaaatctatgctgactgtgacacaagagcctcacatgagaaattaccagtatccaacttcgatactgatagacttgttgatattattattatatgtaatccaattatgaactgtgtgtgtatagagagataataaattcaaaattatgttctcatttttttccctggaactcaataactcatttcactggctctttatcgagagtactagaagttaaattaataaataatgcatttaatgaggcaacagcacttgaaagtttttcattcatcataagaactttatataaaggcattacattggcaaataaggtttggaagcagaagagcaaaaaaaagatattgttaaaatgaggcctccatgcaaaacacatacttccctcccatttatttaactttttttttctcctacctatggggaccaaagtgctttttccttcaggaagtggagatgcatggccatctccccctccctttttccttctcctgcttttctttccccatagaaagtaccttgaagtagcacagcccgtccttgcatgtgcacgagctatcatttgagtaaaagtatacatggagtaaaaatcatattaagcatcagattcaacttatattttctatttcatcttcttcctttcccttctcccaccttctactgggcataattatatcttaatcatatatggaaatgtgcaacatatggtatttgttaaatacgtttgtttttattgcagagcaaaaataaatcaaattagaagcaa

SEQ ID NO: 27 - DNA, 3385 bp, homo sapiens
aagcccctggctgtttatactccggagggtgtccccgtgcgtcatcggtggagtggaccaaaactggtgatctgtttgccctgtgtgaccttgcccagaaccctgctgactgagagaacacatctgctggaagtcctctgggattcaaggtacagggaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctgtcctgtggaggaaggaccaacaccatcaccgcgtggagcagctgaccctgaatggcctcctgcaggctcttcagagcccctgcatcattgaaggggaatctggcaaaggcaagtccactctgctgcagcgaattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccactgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagtttattgacgtctcatgagccagaggaggtgaccaaggggaatggttacttgcagaaaatggtttccatttcggacattacatccacttatagcagcctgctccggtacacctgtgggtcatctgtggaagccaccagggctgttatgaagcacctcgcagcagtgtatcaacacggctgccttctcggactttccatcgccaagaggcctctctggagacaggaatctttgcaaagtgtgaaaaacaccactgagcaagaaattctgaaagccataaacatcaattcctttgtagagtgtggcatccatttatatcaagagagtacatccaaatcagccctgagccaagaatttgaagctttctttcaaggtaaaagcttatatatcaactcagggaacatccccgattacttatttgacttctttgaacatttgcccaattgtgcaagtgccctggacttcattaaactggacttttatgggggagctatggcttcatgggaaaaggctgcagaagacacaggtggaatccacatggaagaggccccagaaacctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagagatgtgctggtgtggctggaagcctcagtttggtcctcagcacctgtaagaacatttattctctcatggtggaagccagtcccctcaccatagaagatgagaggcacatcacatctgtaacaaacctgaaaaccttgagtattcatgacctacagaatcaacggctgccgggtggtctgactgacagcttgggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaagcaattagtgttttttgactttagtactaaagaatttctacctgatccagcattagtcagaaaacttagccaagtgttatccaagttaacttttctgcaagaagctaggcttgttgggtggcaatttgatgatgatgatctcagtgttattacaggtgcttttaaactagtaactgcttaaataaagtgtactcgaagccagtaaaaaaaaaaaaa

SEQ ID NO: 28 - DNA, 764 bp, homo sapiens
agcactgcctggctccacgtgcctcctggtctcagtatggcgctgtcctgggttcttacagtcctgagcctcctacctctgctggaagcccagatcccattgtgtgccaacctagtaccggtgcccatcaccaacgccaccctggaccggatcactggcaagtggttttatatcgcatcggcctttcgaaacgaggagtacaataagtcggttcaggagatccaagcaaccttcttttacttcacccccaacaagacagaggacacgatctttctcagagagtaccagacccgacaggaccagtgcatctataacaccacctacctgaatgtccagcgggaaaatgggaccatctccagatacgtgggaggccaagagcatttcgctcacttgctgatcctcagggacaccaagacctacatgcttgcttttgacgtgaacgatgagaagaactgggggctgtctgtctatgctgacaagccagagacgaccaaggagcaactgggagagttctacgaagctctcgactgcttgcgcattcccaagtcagatgtcgtgtacaccgattggaaaaaggataagtgtgagccactggagaagcagcacgagaaggagaggaaacaggaggagggggaatcctagcaggacacagccttggatcaggacagagacttgggggccatcctgcccctccaacccgacatgtgtacctcagctttttccctcacttgcatcaataaagcttctgtgtttggaacagctaa

SEQ ID NO: 29 - DNA, 2268 bp, homo sapiens
ccctcactcgtcctccgcgcgtcgccgctcttcggttctgctctgtccgccgccatggcccaagctgacatcgcgctgatcggattggccgtcatgggccagaacttaattctgaacatgaatgaccacggctttgtggtctgtgcttttaataggactgtctccaaagttgatgatttcttggccaatgaggcaaagggaaccaaagtggtgggtgcccagtccctgaaagagatggtctccaagctgaagaagccccggcggatcatcctcctggtgaaggctgggcaagctgtggatgatttcatcgagaaattggtaccattgttggatactggtgacatcatcattgacggaggaaattctgaatatagggacaccacaagacggtgccgagacctcaaggccaagggaattttatttgtggggagcggagtcagtggtggagaggaaggggcccggtatggcccatcgctcatgccaggagggaacaaagaagcgtggccccacatcaagaccatcttccaaggcattgctgcaaaagtgggaactggagaaccctgctgtgactgggtgggagatgagggagcaggccacttcgtgaagatggtgcacaacgggatagagtatggggacatgcagctgatctgtgaggcataccacctgatgaaagacgtgctgggcatggcgcaggacgagatggcccaggcctttgaggattggaataagacagagctagactcattcctgattgaaatcacagccaatattctcaagttccaagacaccgatggcaaacacctgctgccaaagatcagggacagcgcggggcagaagggcacagggaagtggaccgccatctccgccctggaatacggcgtacccgtcaccctcattggagaagctgtctttgctcggtgcttatcatctctgaaggatgagagaattcaagctagcaaaaagctgaagggtccccagaagttccagtttgatggtgataagaaatcattcctggaggacattcggaaggcactctacgcttccaagatcatctcttacgctcaaggctttatgctgctaaggcaggcagccaccgagtttggctggactctcaattatggtggcatcgccctgatgtggagagggggctgcatcattagaagtgtattcctaggaaagataaaggatgcatttgatcgaaacccggaacttcagaacctcctactggacgacttctttaagtcagctgttgaaaactgccaggactcctggcggcgggcagtcagcactggggtccaggctggcattcccatgccctgttttaccactgccctctccttctatgacgggtacagacatgagatgcttccagccagcctcatccaggctcagcgggattacttcggggctcacacctatgaactcttggccaaaccagggcagtttatccacaccaactggacaggccatggtggcaccgtgtcatcctcgtcatacaatgcctgatcatgctgctcctgtcaccctccacgattccacagaccaggacattccatgtgcctcatggcactgccacctggccctttgccctattttctgttcagttttttaaaagtgttgtaagagactcctgaggaagacacacagtttatttgtaaagtagctctgtgagagccaccatgccctctgcccttgcctcttgggactgaccaggagctgctcatgtgcgtgagagtgggaaccatctccttgcggcagtggcttccgcgtgccccgtgtgctggtgcggttcccatcacgcagacaggaagggtgtttgcgcactctgatcaactggaacctctgtatcatgcggctgaattccctttttcctttactcaataaaagctacatcagactgatgctctttctccagattcttagtctcacctcggccacatggagccattatccccattggcagaaagatttttctttaaaaaaaaagactagaataacacaagaaaccacatttaggattatgcttcactcagaggaggcaggcagggaggacacaccaggggctttaatacactgggcatgttttctttctccaattgggcaatgggtacatggacgttcactgtaacgtgctttttctttcgtcttttttttttttttttttttttttgctcctggcaagctgtgcgtgacattctttatggctttttgtatgtcaaatacttcatactaaactttctagagaattaaactttaatgatgggctca

SEQ ID NO: 30 - DNA, 708 bp, homo sapiens
agcggtctcccggaccctgccgccctgccactatgtcccgccgctctatgctgcttgcctgggctctccccagcctccttcgactcggagcggctcaggagacagaagacccggcctgctgcagccccatagtgccccggaacgagtggaaggccctggcatcagagtgcgcccagcacctgagcctgcccttacgctatgtggtggtatcgcacacggcgggcagcagctgcaacacccccgcctcgtgccagcagcaggcccggaatgtgcagcactaccacatgaagacactgggctggtgcgacgtgggctacaacttcctgattggagaagacgggctcgtatacgagggccgtggctggaacttcacgggtgcccactcaggtcacttatggaaccccatgtccattggcatcagcttcatgggcaactacatggatcgggtgcccacaccccaggccatccgggcagcccagggtctactggcctgcggtgtggctcagggagccctgaggtccaactatgtgctcaaaggacaccgggatgtgcagcgtacactctctccaggcaaccagctctaccacctcatccagaattggccacactaccgctccccctgaggccctgctgatccgcaccccattcctcccctcccatggccaaaaaccccactgtctccttctccaataaagatgtagctcaaaa

SEQ ID NO: 31 - DNA, 558 bp, homo sapiens
aagaggcctcaaagaaagagctgcggtgcaggaattcgtgtgccggatttggttagctgagcccaccgagaggcgcctgcaggatgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgagcctcccagctcagagtccacgctcctgtgttccgggctgcaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagcgcgtgcacagcgcgggcggaggcggctccaggtccggaggggttgcgggggagctggaaataaacctggagatgatgatgatgatgatgatga

SEQ ID NO: 32 - DNA, 3407 bp, homo sapiens
gctctgctccaggcatctgccacaatgtgggtgcttacacctgctgcttttgctgggaagctcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagagtcgctttcaagataaattctgtcattaatcgtttcaacgtggaagaggacatgacggaacagatcaaagcactaaaccctgtccgctggaaagtgttccagtgcctcttaattgagggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaaaatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttgcctttatgccattgcagtcttgtactttttactgtgatgtacagaaatagtcaacagatgtttccaagaacatatgatatgataatcctaccaattttcaagaagtctctagaaagagataacacatggaaagacggtgtggtgcagcccagcccacggtggctgttccatgaatgctggctacctatgtgtgtggtacctgttgtgtccctttctcttcaaagatcctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatctctgagaagtgacggcacactgggttggcataagatatcctaaaatcacgctggaaccttgggcaaggaagaatgtgagcaagagtagagagagtgcctggatttcatgtcagtgaagccaagtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttagctgcaatttcagtgttcctcactaggtggcatttaaatgtcgcctgatgtcattaagcaccatccaaaaagtctgcttcataatctattttcaagacttggtgattctgaaagttttggtttttgtgactttgtttctcaggaaaaaaaatattcctacttaaattttaagtctataattcaatttaaatatgtgtgtgtctcatccaggataggataggttgtcttctattttccattttacctatttactttttttgtaagaaaagagaaaaatgaattctaaagatgttccccatgggttttgattgtgtctaagctatgatgaccttcatataatcagcataaacataaaacaaattttttacttaacatgagtgcactttactaatcctcatggcacagtggctcacgcctgtaatcccagcacttgggaggacaatgtgggtggatcacgaggtcaggagttcgagaacagcctggccaacatggtgaaaccccgtctccactaaaaatacaaaaattagccaggcatggtggcgtacacttgtaattccagctactcaagaggctgaggcaggaggattgcttgaaccctgaaggcagaggttacagagccaagatagcgccactgcactccagcctggatgacagagcaagactccgtctcaaaaaaaaaaaaaaaaaaaagcaagagagttcaactaagaaaggtcacatatgtgaaagcccaaggacactgtttgatatacagcaggtattcaatcagtgttatttgaaaccaaatctgaatttgaagtttgaatcttctgagttggaatgaatttttttctagctgagggaaactgtatttttctttccccaaagaggaatgtaatgtaaagtgaaataaaactataagctatgttaaataca

SEQ ID NO: 33 - DNA, 485 bp, homo sapiens
cttccttggctcagtgcccttcaccactgctggctttttgctgtagctccacattcctgtgcattgaggggttaacattaggctgggaagatgacaaaacttgaagagcatctggagggaattgtcaatatcttccaccaatactcagttcggaaggggcattttgacaccctctctaagggtgagctgaagcagctgcttacaaaggagcttgcaaacaccatcaagaatatcaaagataaagctgtcattgatgaaatattccaaggcctggatgctaatcaagatgaacaggtcgactttcaagaattcatatccctggtagccattgcgctgaaggctgcccattaccacacccacaaagagtaggtagctctctgaaggctttttacccagcaatgtcctcaatgagggtcttttctttccctcaccaaaacccagccttgcccgtggggagtaagagttaataaacacactcacgaaaa

SEQ ID NO: 34 - DNA, 471 bp, homo sapiens
acattttctcggccctgccagcccccaggaggaaggtgggtctgaatctagcaccatgacggaactagagacagccatgggcatgatcatagacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatagtgttcgtggctgcaatcacgtctgcctgtcacaagtactttgagaaggcaggactcaaatgatgccctggagatgtcacagattcctggcagagccatggtcccaggcttcccaaaagtgtttgttggcaattattcccctaggctgagcctgctcatgtacctctgattaataaatgcttatgaaatga

SEQ ID NO: 35 - DNA, 596 bp, homo sapiens
agagtcactcctgccttcaccatgaagtccagcggcctcttccccttcctggtgctgcttgccctgggaactctggcaccttgggctgtggaaggctctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacacagcaggctttcaagaagtgccagttgatcaatgaataaataaacgagcctatttctctttgca

SEQ ID NO: 36 - DNA, 1763 bp, homo sapiens
gtcttctgtctccgcctcaccctcaggcctgacggtccgagtggagctgcgggacagcccgaacctccaggtcagccccgcggccctccatggcgctggtgcgcgcactcgtctgctgcctgctgactgcctggcactgccgctccggcctcgggctgcccgtggcgcccgcaggcggcaggaatcctcctccggcgataggacagttttggcatgtgactgacttacacttagaccctacttaccacatcacagatgaccacacaaaagtgtgtgcttcatctaaaggtgcaaatgcctccaaccctggcccttttggagatgttctgtgtgattctccatatcaacttattttgtcagcatttgattttattaaaaattctggacaagaagcatctttcatgatatggacaggggatagcccacctcatgttcctgtacctgaactctcaacagacactgttataaatgtgatcactaatatgacaaccaccatccagagtctctttccaaatctccaggttttccctgcgctgggtaatcatgactattggccacaggatcaactgcctgtagtcaccagtaaagtgtacaatgcagtagcaaacctctggaaaccatggctagatgaagaagctattagtactttaaggaaaggtggtttttattcacagaaagttacaactaatccaaaccttaggatcatcagtctaaacacaaacttgtactacggcccaaatataatgacactgaacaagactgacccagccaaccagtttgaatggctagaaagtacattgaacaactctcagcagaataaggagaaggtgtatatcatagcacatgttccagtggggtatctgccatcttcacagaacatcacagcaatgagagaatactataatgagaaattgatagatatttttcaaaaatacagtgatgtcattgcaggacaattttatggacacactcacagagacagcattatggttctttcagataaaaaaggaagtccagtaaattctttgtttgtggctcctgctgttacaccagtgaagagtgttttagaaaaacagaccaacaatcctggtatcagactgtttcagtatgatcctcgtgattataaattattggatatgttgcagtattacttgaatctgacagaggcgaatctaaagggagagtccatctggaagctggagtatatcctgacccagacctacgacattgaagatttgcagccggaaagtttatatggattagctaaacaatttacaatcctagacagtaagcagtttataaaatactacaattacttctttgtgagttatgacagcagtgtaacatgtgataagacatgtaaggcctttcagatttgtgcaattatgaatcttgataatatttcctatgcagattgcctcaaacagctttatataaagcacaattactagtatttcacagtttttgctaatagaaaatgctgattctgattctgagatcaatttgtgggaattttacataaatctttgttaattactgagtgggcaagtagacttcctgtctttgctttctttttttttttctttttgatgccttaatgtagatatctttatcattctgaattgtattatatatttaaagtgctcattaatagaatgatggatgtaaattggatgtaaatattcagtttatataattatatctaatttgtacccttgttgaaattgtcatttatacaataaagcgaattctttatctctaaa

SEQ ID NO: 37 - DNA, 836 bp, homo sapiens
aactcctgccaggcagtgcccttcccggagcgtgccctcgccgctgagctcccctgaacagcagctgcagcagccatggccccgccctgggtgcccgccatgggcttcacgctggcgcccagcctggggtgcttcgtgggctcccgctttgtccacggcgagggtctccgctggtacgccggcctgcagaagccctcgtggcacccgccccactgggtgctgggccctgtctggggcacgctctactcagccatggggtacggctcctacctggtctggaaagagctgggaggcttcacagagaaggctgtggttcccctgggcctctacactgggcagctggccctgaactgggcatggccccccatcttctttggtgcccgacaaatgggctgggccttggtggatctcctgctggtcagtggggcggcggcagccactaccgtggcctggtaccaggtgagcccgctggccgcccgcctgctctacccctacctggcctggctggccttcacgaccacactcaactactgcgtatggcgggacaaccatggctggcgtgggggacggcggctgccagagtgagtgcccggcccaccagggactgcagctgcaccagcaggtgccatcacgcttgtgatgtggtggccgtcacgctttcatgaccactgggcctgctagtctgtcagggccttggcccaggggtcagcagagcttcagaggtggccccacctgagcccccacccgggagcagtgtcctgtgctttctgcatgcttagagcatgttcttggaacatggaattttataagctgaataaagtttttgacttccttta

SEQ ID NO: 38 - DNA, 3853 bp, homo sapiens
agcactcattggacttcagcatgactactcagttgccagcttacgtggcaattttgcttttctatgtctcaagagccagctgccaggacactttcactgcagctgtttatgagcatgcagcgatattgcccaatgccaccctaacaccagtgtctcgtgaggaggctttggcattaatgaatcggaatctggacattttggaaggagcgatcacatcagcagcagatcagggtgcgcatattattgtgactccagaagatgctatttatggctggaacttcaacagggactctctctacccatatttggaggacatcccagaccctgaagtaaactggatcccctgtaataatcgtaacagatttggccagaccccagtacaagaaagactcagctgcctggccaagaacaactctatctatgttgtggcaaatattggggacaagaagccatgcgataccagtgatcctcagtgtccccctgatggccgttaccaatacaacactgatgtggtatttgattctcaaggaaaactggtggcacgctaccataagcaaaaccttttcatgggtgaaaatcaattcaatgtacccaaggagcctgagattgtgactttcaataccacctttggaagttttggcattttcacatgctttgatatactcttccatgatcctgctgttaccttggtgaaagatttccacgtggacaccatagtattcccaacagcttggatgaatgttttgccacatttgtcagctgttgaattccactcagcttgggctatgggcatgagggtcaatttccttgcatccaacatacattacccctcaaagaaaatgacaggaagtggcatctatgcacccaattcttcaagagcatttcattatgatatgaagacagaagagggaaaactcctcctctcgcaactggattcccacccatcccattctgcagtggtgaactggacttcctatgccagcagtatagaagcgctctcatcaggaaacaaggaatttaaaggcactgtctttttcgatgaattcacttttgtgaagctcacaggagttgcaggaaattatacagtttgtcagaaagatctctgctgtcatttaagctacaaaatgtctgagaacataccaaatgaagtgtacgctctaggggcatttgacggactgcacactgtggaagggcgctattatctacagatttgtaccctgttgaaatgtaaaacgactaatttaaacacttgcggtgactcagctgaaacagcttctaccaggtttgaaatgttctccctcagtggcactttcggaacccagtatgtctttcctgaggtgttgctgagtgaaaatcagcttgcacctggagaatttcaggtgtcaactgacggacgcttgtttagtctgaagccaacatccggacctgtcttaacagtaactctgtttgggaggttgtatgagaaggactgggcatcaaatgcttcatcaggcctcacagcacaagcaagaataataatgctaatagttatagcacctattgtatgctcattaagttggtagaatattgactttttctcttttttatttgggataatttaaaaaatgatggatgagaaaagaaagattggtccgggttaatattatcctctagtataagtgaattactagtttctctttatttagacaaacacacacacaccagataatataaacttaataaattatctgttaatgtagattttatttaaaaaactatatttgaacattggtctttcttggacgtgagctaattatatcaaataagtatcacaaatcttttacgcagaagaaataaaaactacgggtagaaaacataagaactatcataaaatttacttacaaggaggctgctcttgttaccacttttattatattacgtatcacttattcagctctgctgaaaatttccaatgactttgtttgtttgctctttttgttttttacctaaacaatacattttgattctcttgtgggttgataatgtctccccaaaatttacatgttgaagcacctcagaatgtgactgtatttggagacagggtctttaaagaggtaaaataaggtcattaggatagaccctaattcaatatgactgatgatcataaaagaagaggcgagtagggcacaacaggcacaaagggagaccataaggagacacagaggaaggacaactctttacaagctaagaagagagggcctcagaagaaaccaaccctgccaacaccttgatcttggacttccagcctccaaaactatgagaaataaatttctattgtttaagtcacccagtccatggtactttgttaggcagccctggcaaatgaatcaaagacccattcctgttcctctccccaccactactgttttctactgtaatctgaagcttcaacaaaaggcttacctggtaagaatattcagctggtctgggtcctcaagactccaatagacactcttagagaaggattgctgatggattgatagtgaaaccattagatcattgaattcctctggaattagaaaaccagagagtcccattttaagaaattagatatttaatatagcattgtgtgttctattttagtaacagcagaatctcttgacattacacaactcagtgaaacaacatcatttaagccaaaatatctcccaactgactgatagactctgagcactaatatcatagtgctgtgatgatggacaattacatagtaccgataacagccatgcactgtgcaaagcatgcccttctgcacaggagagcaaggcacttgcagtagtgatctatgccagcaaaacatcattttgagacaaacatttttgtggcagatgtttttcctaaaaagtactatatcatccaagaaatatttgagtaaaatcccttgttcttttgggtgacattaactgacatttgctttttttcaagacctaatagaaaataagaaagcccataatgtatttagaaacaggaatcctcagagcaattctctgtattctcatataatttcaatgtaaaacagaaaacatattgatgtgttggtgataggcttgaattattaaaaacttcaaaaacatcctaagtgtttcttttttgctcaacgttgtcaactatagtaggtctcccttgtggtgtaatgaattgcccccaaactattatcttaaaacaacaaacatttattatcttatagcatttctgagggtcaggatctgggactggcttagtggagttgttctggatcagggcctttggaaagttgtagttaacttgtccccagggctgccatcatctcaaggctcgggtggggctggagaaaatctgcttctcagctcactcacggcggttgccaggcctccattctttaggatgctagaaaaactttcataaaatgtcatctggcttctcctagagcaatgatactgagagagaaagcacatgagagaaagagcgagggaacttggatgtaagccacagtctttgaaaacctaatcacagaagtgacatctcttcttccacatgatgttggtcacatggaccaacaatggcacaacgtggacagaatcaaacagagttgagaatatcaggaggtggggcttcatgggggccattttggatgctatcatagtgaatatatgtatttatatttatatctgtatatattgcaatgtaatttaaaaaataggattgttttccttttctttttgctatatgtgatatgtatttcaaaatacactcccaatagttacgtctgaaaagcactacactaaaaaactttctatacattgaataattaaattaaataatctaataatctcta

SEQ ID NO: 39 - DNA, 1308 bp, homo sapiens
gccgtggccatggagctctggaagctgggctgggggaggaagcctggtggctctgacctcccctggaggcgaaggaggcccagccatgacactcttaacggatgccacgccgctggtgaaggagccccatcccctgcctctggtcccacgtccctggttcctccctagcctctttgctgccttcaatgtggtgctgctggtctttttcagtggcctcttcttcgcattcccttgcaggtggctggctcagaacggggagtgggcctttcctgttatcacaggctccctctttgtccttaccttcttcagtcttgtttcactcaacttctcagaccctggcatcttacatcaaggctccgctgagcagggccccttgacggtgcacgtggtgtgggtgaaccacggggccttccgcctgcaatggtgtccaaagtgctgcttccaccgcccgccccggacttaccactgcccctggtgcaacatctgtgtggaggactttgaccaccactgcaagtgggtcaataactgcatcggtcaccgcaacttccgcttcttcatgctgcttgtcctgtccctgtgcctctactcgggcgccatgctggtcacctgtctcatcttcctggtgcgcacaacccacctgcccttctccaccgacaaggccatcgccatcgtggtggccgtgtccgccgcgggcctcctggtgccgctgtccctcctgctgctgatccaggcactgtccgtgagctcggccgaccgcacctacaagggcaagtgcagacaccttcagggatacaaccccttcgaccagggctgtgccagcaactggtatttaacaatttgtgcaccactgggacccaagtacatggctgaagctgtccagctgcagagagtggtggggcctgactggacatccatgccgaatctgcaccctccaatgtccccctctgctctcaaccccccagccccaacctctgggtccctacaaagcagggaagggacccccggggcgtggtgaggctgcagctctccaggagttccacacgggcccagtgctgcccctgctgctgcaggagcccccaggcgaggttcggccttcctctcgcccctgtgcacccggagatgcccacagcaccagcacctgagctcacctccgaacccgcctcctgaacccgcctcctgaacctgcctccttacctcccacttcctgagccctgagtggaagcctttctgtgccttgccctttgcccactcccctggtgggactgccaagaccctcaatgcccattaaatactcttgcctgcctctta

(19) United States
(12) Patent Application Publication
(10) Pub. No.: US 2024/0026449 A1
(43) Pub. Date: Jan. 25, 2024

(54) RNA MARKERS FOR DIAGNOSING INFECTIONS

(71) Applicant: MeMed Diagnostics Ltd., Tirat HaCarmel (IL)

(72) Inventors: Oded SHAHAM, Haifa (IL); Niv Steven MASTBOIM, Haifa (IL); Oren ZARCHIN, Kfar Saba (IL); Roy NAVON, Tel-Aviv (IL); Eran EDEN, Haifa (IL); Kfir OVED, Hof HaCarmel (IL)

(73) Assignee: MeMed Diagnostics Ltd., Tirat HaCarmel (IL)

(21) Appl. No.: 17/916,577

(22) PCT Filed: Apr. 8, 2021
(86) PCT No.: PCT/IL2021/050410

§ 371 Date: Oct. 2, 2022

Related U.S. Application Data
(60) Provisional application No. 63/006,758, filed on Apr. 8, 2020.
(60) Provisional application No. 63/014,214, filed on Apr. 23, 2020.
(60) Provisional application No. 63/030,937, filed on May 28, 2020.
(60) Provisional application No. 63/085,189, filed on Sep. 30, 2020.
(60) Provisional application No. 63/130,946, filed on Dec. 28, 2020.

(51) Int. Cl. C12Q 1/6883; C12Q 1/70; C12Q 1/689

(52) U.S. Cl. CPC C12Q 1/6883; C12Q 1/70; C12Q 1/689; C12Q 2600/158

(57) ABSTRACT

Methods of determining infection type are disclosed based on the amount of CD177 RNA and the amount of IFI44L RNA in a sample obtained from the subject. Additional RNA markers for determining infection type are also disclosed. Kits capable of determining infection type are also disclosed.

### RELATED APPLICATIONS

This application claims the benefit of priority of U.S. Provisional Applications 63/006,758 filed 8 Apr. 2020, 63/014,214 filed 23 Apr. 2020, 63/030,937 filed 28 May 2020, 63/085,189 filed 30 Sep. 2020 and 63/130,946 filed 28 Dec. 2020, the contents of which are incorporated herein by reference in their entirety.

### SEQUENCE LISTING STATEMENT

The ASCII file, entitled 86662 Sequence Listing.txt, created in 6 Apr. 2021, comprising 120,860 bytes, submitted concurrently with the filing of this application is incorporated herein by reference.

### FIELD AND BACKGROUND OF THE INVENTION

The present invention, in some embodiments thereof, relates to the identification of signatures and determinants associated with bacterial and viral infections. More specifically it was discovered that certain RNA determinants are differentially expressed in a statistically significant manner in subjects with bacterial and viral infections.

Antibiotics are the world's most prescribed class of drugs with a 25-30 billion $US global market. Antibiotics are also the world's most misused drug with a significant fraction of all drugs (40-70%) being wrongly prescribed (Linder and Stafford 2001; Scott and Cohen 2001; Davey, P. and E. Brown, et al 2006; Cadieux, G. and R. Tamblyn, et al. 2007; Pulcini, C. and E. Cua, et al. 2007)′(“CDC—Get Smart: Fast Facts About Antibiotic Resistance” 2011).

One type of antibiotics misuse is when the drug is administered in case of a non-bacterial disease, such as a viral infection, for which antibiotics is ineffective. For example, according to the USA center for disease control and prevention CDC, over 60 Million wrong antibiotics prescriptions are given annually to treat flu in the US. The health-care and economic consequences of the antibiotics over-prescription include: (i) the cost of antibiotics that are unnecessarily prescribed globally, estimated at >$10 billion annually; (ii) side effects resulting from unnecessary antibiotics treatment are reducing quality of healthcare, causing complications and prolonged hospitalization (e.g. allergic reactions, Antibiotics-associated diarrhea, intestinal yeast etc.) and (iii) the emergence of resistant strains of bacteria as a result of the overuse.

Resistance of microbial pathogens to antibiotics is increasing world-wide at an accelerating rate (“CDC—Get Smart: Fast Facts About Antibiotic Resistance” 2013; “European Surveillance of Antimicrobial Consumption Network (ESAC-Net)” 2014; “CDC—About Antimicrobial Resistance” 2013; “Threat Report 2013|Antimicrobial Resistance|CDC” 2013), with a concomitant increase in morbidity and mortality associated with infections caused by antibiotic resistant pathogens (“Threat Report 2013|Antimicrobial Resistance|CDC” 2013). At least 2 million people are infected with antibiotic resistant bacteria each year in the US alone, and at least 23,000 people die as a direct result of these infections (“Threat Report 2013|Antimicrobial Resistance|CDC” 2013). In the European Union, an estimated 400,000 patients present with resistant bacterial strains each year, of which patients die (“WHO Europe—Data and Statistics” 2014). Consequently, the World Health Organization has warned that therapeutic coverage will be insufficient within 10 years, putting the world at risk of entering a “post-antibiotic era”, in which antibiotics will no longer be effective against infectious diseases (“WHO|Antimicrobial Resistance” 2013). The CDC considers this phenomenon “one of the world's most pressing health problems in the 21^{st} century” (“CDC—About Antimicrobial Resistance” 2013; Arias and Murray 2009).

Antibiotics under-prescription is not uncommon either. For example up to 15% of adult bacterial pneumonia hospitalized patients in the US receive delayed or no Abx treatment, even though in these instances early treatment can save lives and reduce complications (Houck, P. M. and D. W. Bratzler, et al 2002).

Technologies for infectious disease diagnostics have the potential to reduce the associated health and financial burden associated with antibiotics misuse. Ideally, such a technology should: (i) accurately differentiate between a bacterial and viral infections; (ii) be rapid (within minutes); (iii) be able to differentiate between pathogenic and non-pathogenic bacteria that are part of the body's natural flora; (iv) differentiate between mixed co-infections and pure viral infections and (v) be applicable in cases where the pathogen is inaccessible (e.g. sinusitis, pneumonia, otitis-media, bronchitis, etc).

Current solutions (such as culture, PCR and immunoassays) do not fulfill all these requirements: (i) Some of the assays yield poor diagnostic accuracy (e.g. low sensitivity or specificity) (Uyeki et al. 2009), and are restricted to a limited set of bacterial or viral strains; (ii) they often require hours to days; (iii) they do not distinguish between pathogenic and non-pathogenic bacteria (Del Mar, C 1992), thus leading to false positives; (iv) they often fail to distinguish between a mixed and a pure viral infections and (v) they require direct sampling of the infection site in which traces of the disease causing agent are searched for, thus prohibiting the diagnosis in cases where the pathogen resides in an inaccessible tissue, which is often the case. Moreover, currently available diagnostic approaches often suffer from reduced clinical utility because they do not distinguish between pathogenic strains of microorganisms and potential colonizers, which can be present as part of the natural microbiota without causing an infection (Kim, Shin, and Kim 2009; Shin, Han, and Kim 2009; Jung, Lee, and Chung 2010; Rhedin et al. 2014). For example, Rhedin and colleagues recently tested the clinical utility of qPCR for common viruses in acute respiratory illness (Rhedin et al. 2014). The authors concluded that qPCR detection of several respiratory viruses including rhinovirus, enterovirus and coronavirus should be interpreted with caution due to high detection rates in asymptomatic children. Other studies reached similar conclusions after analyzing the detection rates of different bacterial strains in asymptomatic patients (Bogaert, De Groot, and Hermans 2004; Spuesens et al. 2013).

Consequentially, there is still a diagnostic gap, which in turn often leads physicians to either over-prescribe Abx (the “Just-in-case-approach”), or under-prescribe Abx (the “Wait-and-see-approach”) (Little, P. S. and I. Williamson 1994; Little, P. 2005; Spiro, D. M. and K. Y. Tay, et al 2006), both of which have far reaching health and financial consequences.

Accordingly, a need exists for a rapid method that accurately differentiates between bacterial, viral, mixed (bacterial and viral co-infection) and non-infectious disease patients that addresses these challenges. An approach that has the potential to address these challenges relies on monitoring the host's immune-response to infection, rather than direct pathogen detection (Cohen et al. 2015). Bacterial-induced host proteins such as procalcitonin, C-reactive protein (CRP), and Interleukin-6, are routinely used to support diagnosis of infection. However, their performance is negatively affected by inter-patient variability, including time from symptom onset, clinical syndrome, and pathogen species (Tang et al. 2007; Limper et al. 2010; Engel et al. 2012; Quenot et al. 2013; van der Meer et al. 2005; Falk and Fahey 2009). Oved et al. 2015 has developed an immune signature, combining both bacterial- and viral-induced circulating host-proteins, which can aid in the correct diagnosis of patients with acute infections.

Additional background art includes Ramilo et al., Blood, Mar. 1, 2007, Vol 109, No. 5, pages 2066-2077, Zaas et al., Sci Transl Med. 2013 Sep. 18; 5(203) 203ra126. doi:10.1126/scitranslmed.3006280; Almansa, R. et al. Transcriptomic correlates of organ failure extent in sepsis. J. Infect. 70, 445-456 (2015); Balamuth, F. et al. Gene Expression Profiles in Children With Suspected Sepsis. Ann. Emerg. Med. 75, 744-754 (2020); Cameron, M. J. et al. Interferon-mediated immunopathological events are associated with atypical innate and adaptive immune responses in patients with severe acute respiratory syndrome. J. Virol. 81, 8692-8706 (2007); Davenport, E. E. et al. Genomic landscape of the individual host response and outcomes in sepsis: a prospective cohort study. Lancet Respir. Med. 4, 259-271 (2016); Dunning, J. et al. Progression of whole-blood transcriptional signatures from interferon-induced to neutrophil-associated patterns in severe influenza. Nat. Immunol. 19, 625-635 (2018); Hoang, L. T. et al. Patient-based transcriptome-wide analysis identify interferon and ubiquination pathways as potential predictors of influenza A disease severity. PloS One 9, e111640 (2014); Lill, M. et al. Peripheral blood RNA gene expression profiling in patients with bacterial meningitis. Front. Neurosci. 7, 33 (2013); Navon, R. et al. Novel rank-based statistical methods reveal microRNAs with differential expression in multiple cancer types. PloS One 4, e8003 (2009); Parnell, G. et al. Aberrant cell cycle and apoptotic changes characterise characterize severe influenza A infection—a meta-analysis of genomic signatures in circulating leukocytes. PloS One 6, e17186 (2011); Parnell, G. P. et al. Identifying key regulatory genes in the whole blood of septic patients to monitor underlying immune dysfunctions. Shock Augusta Ga 40, 166-174 (2013); Scicluna, B. P. et al. A molecular biomarker to diagnose community-acquired pneumonia on intensive care unit admission. Am. J. Respir. Crit. Care Med. 192, 826-835 (2015); Sweeney, T. E. et al. A community approach to mortality prediction in sepsis via gene expression analysis. Nat. Commun. 9, 694 (2018); Tang, B. M. et al. Neutrophils-related host factors associated with severe disease and fatality in patients with influenza infection. Nat. Commun. 10, 3422 (2019); Tsalik, E. L. et al. An integrated transcriptome and expressed variant analysis of sepsis survival and death. Genome Med. 6, 111 (2014); Wong, H. R. et al. Genome-level expression profiles in pediatric septic shock indicate a role for altered zinc homeostasis in poor outcome. Physiol. Genomics 30, 146-155 (2007); US Patent Application No. 20080171323, WO2011/132086, WO2013/117746 and WO2017/149548, WO2017/149547.

### SUMMARY OF THE INVENTION

According to an aspect of the present invention there is provided a method of ruling in a viral infection in a test subject comprising:
(a) measuring the amount of CD177 RNA and the amount of IFI44L RNA in a sample obtained from the subject;
(b) generating a score based on the amount of CD177 RNA and the amount of IFI44L RNA, wherein the score is an increasing function of the amount of CD177 RNA and a decreasing function of the amount of IFI44L RNA, wherein when the score is below a predetermined level a viral infection is ruled in, wherein the predetermined level is based on the amount of CD177 RNA and the amount of IFI44L RNA in bacterial subjects.

According to an aspect of the present invention there is provided a method of ruling in a bacterial infection in a test subject comprising:
(a) measuring the amount of CD177 RNA and the amount of IFI44L RNA in a sample obtained from the subject;
(b) generating a score based on the amount of CD177 RNA and the amount of IFI44L RNA, wherein the score is an increasing function of the amount of CD177 RNA and a decreasing function of the amount of IFI44L RNA, wherein when the score is above a predetermined level a bacterial infection is ruled in, wherein the predetermined level is based on the amount of CD177 RNA and the amount of IFI44L RNA in viral subjects.

According to an aspect of the present invention there is provided a method of ruling in a viral infection in a test subject comprising:
(a) measuring the amount of CD177 RNA and the amount of IFI44L RNA in a sample obtained from the subject;
(b) generating a score based on the amount of CD177 RNA and the amount of IFI44L RNA, wherein the score is an increasing function of the amount of CD177 RNA and an increasing function of the amount of IFI44L RNA, wherein when the score is above a predetermined level a viral infection is ruled in, wherein the predetermined level is based on the amount of CD177 RNA and the amount of IFI44L RNA in non-infectious subjects.

According to embodiments of the present invention, the score is a monotonically increasing function of the amount of CD177 RNA and a monotonically decreasing function of the amount of IFI44L RNA.

According to embodiments of the present invention, the function is a linear function.

According to embodiments of the present invention, the method further comprises measuring the amount of at least one additional RNA selected from the group consisting of MMP9 RNA, IFIT1 RNA and PI3 RNA in a sample of the subject.

According to embodiments of the present invention, the method further comprises measuring the amount of IFIT1 RNA in a sample of the subject.

According to embodiments of the present invention, the method further comprises measuring the amount of each of MMP9 RNA, IFIT1 RNA and PI3 RNA in a sample of the subject.

According to embodiments of the present invention, the score is based on the amount of the CD177 RNA the IFI44L RNA the IFIT1 RNA the MMP9 RNA and the PI3 RNA.

According to an aspect of the present invention there is provided a method of ruling in a viral infection in a test subject comprising measuring the amount of CD177 RNA and IFI44L RNA in a sample derived from the subject, wherein when the amount of the CD177 RNA is below a predetermined level and the amount of the IFI44L RNA is above a predetermined level, the infection is a viral infection, wherein the predetermined level is based on the amount of the CD177 RNA and the amount of the IFI44L RNA in subjects with a bacterial infection.

According to an aspect of the present invention there is provided a method of ruling in a bacterial infection in a test subject comprising measuring the amount of CD177 RNA and IFI44L RNA in a sample derived from the subject, wherein when the amount of the CD177 RNA is above a predetermined level and the amount of the IFI44L RNA is below a predetermined level, the infection is a bacterial infection, wherein the predetermined level is based on the amount of the CD177 RNA and the amount of the IFI44L RNA in subjects with a viral infection.

According to an aspect of the present invention there is provided a method of ruling in a viral infection in a test subject comprising measuring the amount of CD177 RNA and IFI44L RNA in a sample derived from the subject, wherein when the amount of the CD177 RNA is above a predetermined level and the amount of the IFI44L RNA is above a predetermined level, the infection is a viral infection, wherein the predetermined level is based on the amount of the CD177 RNA and the amount of the IFI44L RNA in non-infectious subjects.

According to embodiments of the present invention, the method further comprises measuring the amount of IFIT1 RNA in a sample of the subject, wherein when the amount of the CD177 RNA is below a predetermined level, the amount of the IFI44L RNA is above a predetermined level and the amount of IFIT1 is above a predetermined threshold, the infection is a viral infection.

According to embodiments of the present invention, the method further comprises measuring the amount of IFIT1 RNA in a sample of the subject, wherein when the amount of the CD177 RNA is above a predetermined level, the amount of the IFI44L is below a predetermined level, and the amount of IFIT1 RNA is below a predetermined level, the infection is a bacterial infection.

According to embodiments of the present invention, the method further comprises measuring the amount of MMP9 RNA and PI3 RNA in a sample of the subject, wherein the amount of CD177 RNA, IFI44L RNA, IFIT1 RNA, MMP9 RNA and PI3 RNA is used to determine infection type.

According to embodiments of the present invention, the method further comprises measuring the amount of MMP9 RNA and PI3 RNA in a sample of the subject, when the amount of the CD177 RNA is below a predetermined level, the IFI44L RNA is above a predetermined level, the IFIT1 RNA is above a predetermined level, the MMP9 RNA is below a predetermined level and the PI3 RNA is below a predetermined level the infection is a viral infection, wherein the predetermined levels are based on subjects with a bacterial infection.

According to embodiments of the present invention, the method further comprises measuring the amount of MMP9 RNA and PI3 RNA in a sample of the subject, when the amount of the CD177 RNA is above a predetermined level, the IFI44L RNA is below a predetermined level, the IFIT1 RNA is below a predetermined level, the MMP9 RNA is above a predetermined level and the PI3 RNA is above a predetermined level the infection is a bacterial infection, wherein the predetermined levels are based on subjects with a viral infection.

According to an aspect of the present invention there is provided a method of diagnosing the severity of a viral infection of a test subject, comprising measuring the expression level of a first RNA set forth in Table 5 and the level of a second RNA set forth in Table 6 in a sample of the subject, wherein the levels are indicative of the severity of the viral infection.

According to embodiments of the present invention, the method further comprising measuring the expression level of a third RNA set forth in Table 7.

According to an aspect of the present invention there is provided a method of diagnosing the severity of a test subject with an infectious disease, comprising measuring the expression level of pairs of RNAs set forth in Table 8 or triplets of RNAs set forth in Table 9 in a sample of the subject, wherein the levels are indicative of the severity of the infectious disease.

According to an aspect of the present invention there is provided a method of diagnosing the severity of a test subject with an infectious disease, comprising measuring the expression level of:
(i) CEACAM8, MMP8, SAMSN1 and TGFBI; or
(ii) IL1R2, MMP8, PRC1 and CD74; or
(iii) DEFA4, IL1R2, MMP8, RETN and LY86, in a sample of the subject, wherein the levels are indicative of the severity of the infectious disease.

According to an aspect of the present invention there is provided a method of diagnosing the severity of a viral infection of a test subject, comprising measuring the expression level of pairs of RNAs set forth in Table 11 or triplets of RNAs set forth in Table 12 in a sample of the subject, wherein the levels are indicative of the severity of the viral infection.

According to an aspect of the present invention there is provided a method of diagnosing the severity of a bacterial disease of a test subject, comprising measuring the expression level of pairs of RNAs set forth in Table 14 or triplets of RNAs set forth in Table 15 in a sample of the subject, wherein the levels are indicative of the severity of the bacterial infection.

According to embodiments of the present invention, the test subject does not have a chronic disease.

According to embodiments of the present invention, the test subject shows symptoms of an infection.

According to embodiments of the present invention, the symptoms are selected from the group consisting of fever, cough, sputum production, myalgia, fatigue, headache, anorexia, dyspnea, diarrhea, nausea, dizziness, headache, vomiting, abdominal pain, sore throat, nasal congestion, hemoptysis and chills.

According to embodiments of the present invention, the test subject is asymptomatic of an infection.

According to embodiments of the present invention, the non-infectious subjects are healthy subjects.

According to embodiments of the present invention, the method further comprising measuring the amount of at least one additional RNA marker listed in Table 3.

According to embodiments of the present invention, the level of no more than 10 RNA markers is used to determine the infection type.

According to embodiments of the present invention, no more than 10 RNA markers are measured to determine the infection type.

According to embodiments of the present invention, the level of no more than 5 RNA markers is used to determine the infection type.

According to embodiments of the present invention, no more than 5 RNA markers are measured to determine the infection type.

According to embodiments of the present invention, the measuring is carried out no more than 48 hours after symptom onset.

According to embodiments of the present invention, the sample is whole blood or a fraction thereof.

According to embodiments of the present invention, the fraction comprises cells selected from the group consisting of lymphocytes, monocytes and granulocytes.

According to embodiments of the present invention, the fraction comprises serum or plasma.

According to embodiments of the present invention, the virus of the viral infection is selected from the group consisting of Adenovirus, Bocavirus, Coronavirus, Enterovirus, Influenza virus, Metapneumovirus, Parainfluenza virus, Respiratory syncytial virus and Rhinovirus.

According to embodiments of the present invention, the coronavirus is severe acute respiratory syndrome coronavirus (SARS-CoV-2) and Middle East respiratory syndrome coronavirus (MERS-CoV).

According to embodiments of the present invention, the coronavirus is SARS-CoV-2.

According to embodiments of the present invention, the sample is a blood sample.

According to an aspect of the present invention there is provided a kit for diagnosing an infection type comprising RNA detection reagents which specifically detect CD177 RNA and IFI44L RNA.

According to embodiments of the present invention, the kit further comprises RNA detection reagents which specifically detect IFIT1 RNA.

According to embodiments of the present invention, the kit further comprises RNA detection reagents which specifically detect at least one additional RNA selected from the group consisting of IFIT1 RNA, MMP9 RNA and PI3 RNA.

According to embodiments of the present invention, the kit further comprises RNA detection reagents which specifically detect IFIT1 RNA, MMP9 RNA and PI3 RNA.

According to embodiments of the present invention, the kit further comprises RNA detection reagents which specifically detect an RNA marker set forth in Table 3.

According to embodiments of the present invention, the RNA detection reagents are attached to a detectable moiety.

According to embodiments of the present invention, the RNA detection reagents comprises at least one polynucleotide that specifically hybridizes to CD177 RNA and at least one polynucleotide that specifically hybridizes to IFI44L RNA.

According to embodiments of the present invention, the kit comprises detection reagents that specifically detect no more than 10 RNA markers.

According to embodiments of the present invention, the kit comprises detection reagents that specifically detect no more than 5 RNA markers.

According to embodiments of the present invention, the 5 RNAs comprise CD177 RNA, IFI44L RNA, IFIT1 RNA, MMP9 RNA and PI3 RNA.

According to an aspect of the present invention there is provided a method of treating a viral infection of a subject in need thereof comprising:
(a) ruling in a viral infection in the subject according to any one of claim 1, 3, 10, 12, 18 or 22;
(b) administering to the subject a therapeutically effective amount of an antiviral agent, thereby treating the viral infection of the subject.

According to an aspect of the present invention there is provided a method of treating a bacterial infection of a subject in need thereof comprising:
(a) ruling in a bacterial infection in the subject according to claim 2, 11 or 23;
(b) administering to the subject a therapeutically effective amount of an antibiotic, thereby treating the bacterial infection of the subject.

According to embodiments of the present invention, the subject shows symptoms of an infectious disease.

According to embodiments of the present invention, the symptoms comprise fever.

According to an aspect of the present invention there is provided a method of identifying an infection outbreak or a change in virulence of an existing pathogen in a medical facility, the method comprising:
obtaining a distribution pertaining to expression values of TRAIL protein, IP10 protein, and CRP protein derived from each of a plurality of patients in the medical facility;
accessing a computer readable medium storing comparative data;
comparing the calculated distribution to the comparative data; and
issuing an alert that an infection is expected to outbreak across the medical facility, or that a change in virulence of the existing pathogen in the medical facility is expected to occur, when the comparison indicates a rise in the distribution above a predetermined threshold, and issuing a report pertaining to the comparison otherwise.

According to an aspect of the present invention there is provided a method of identifying an infection outbreak or a change in virulence of an existing pathogen in a medical facility, the method comprising:
obtaining a distribution pertaining to expression values of at least one of PCT protein and IL6 protein from each of a plurality of patients in the medical facility;
accessing a computer readable medium storing comparative data;
comparing the calculated distribution to the comparative data; and
issuing an alert that an infection is expected to outbreak across the medical facility, or that a change in virulence of the existing pathogen in the medical facility is expected to occur, when the comparison indicates a rise in the distribution above a predetermined threshold, and issuing a report pertaining to the comparison otherwise.

According to embodiments of the present invention, the method comprises receiving the expression values of the proteins, and wherein the obtaining comprises calculating the distribution.

According to embodiments of the present invention, the distribution pertaining to the expression values is a distribution of a classification score calculated based on the expression values.

According to embodiments of the present invention, the method comprises calculating the classification score.

According to embodiments of the present invention, the method further comprises obtaining a distribution pertaining to expression values of at least one additional determinant selected from the group consisting of determinants listed in Table 34, wherein the accessing the computer readable medium and the comparing is also executed with respect to the at least one additional determinant.

According to embodiments of the present invention, the distribution is separate for each protein.

According to embodiments of the present invention, the distribution is a combined distribution for all proteins.

According to embodiments of the present invention, the comparative data comprises history data pertaining to previously received expression values of the proteins within the medical facility. According to embodiments of the present invention, the method is executed at a central facility remote to the medical facility, wherein the method comprises transmitting the alert to a receiving computer at the medical facility.

According to embodiments of the present invention, the method is executed for a plurality of medical facilities, wherein the transmitting is separate to each medical facility.

According to embodiments of the present invention, the method comprises analyzing changes in the distribution across the medical facilities and estimating a spread of the infection outbreak based on the analysis.

According to embodiments of the present invention, the method comprises calculating an outbreak score for each medical facility based on the comparison, and allocating treatment resources among the plurality of medical facilities based of the outbreak score.

According to embodiments of the present invention, the method comprises applying quarantine to the medical facility upon determination that an infection is expected to outbreak across the medical facility.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for predicting an infection outbreak or a change in virulence of an existing outbreak in a medical facility, according to various exemplary embodiments of the present invention;

FIG. 2 is a schematic illustration of a client-server computer configuration according to some embodiments of the present invention;

FIG. 3 is a 3-dimensional graph showing combined median of protein levels, measured in six different hospitals, according to some embodiments of the present invention;

FIG. 4 is a flowchart diagram of a method suitable for analyzing biological data obtained from a subject, according to various exemplary embodiments of the present invention.

FIG. 5 is a schematic illustration describing a procedure for calculating a distance of a curved line from an axis according to some embodiments of the present invention.

FIGS. 6A-D are schematic illustrations describing a procedure for obtaining the smooth version of a segment of a curved line, according to some embodiments of the present invention.

### DESCRIPTION OF SPECIFIC EMBODIMENTS OF THE INVENTION

The present invention, in some embodiments thereof, relates to the identification of signatures and determinants associated with bacterial and viral infections. More specifically it was discovered that certain RNA determinants are differentially expressed in a statistically significant manner in subjects with bacterial and viral infections.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Methods of distinguishing between bacterial and viral infections by analyzing RNA determinants have been disclosed in International Patent Application WO2017/149548 and WO2017/149547, to the present inventors. Seeking to expand the number and type of determinants that can aid in accurate diagnosis, the present inventors have now carried out additional clinical experiments and have identified other determinants that can be used for this aim.

Correct diagnosis of bacterial patients is of high importance as these patients require antibiotic treatment and in some cases more aggressive management (hospitalization, additional diagnostic tests etc). Misclassification of bacterial patients increases the chance of morbidity and mortality. Therefore, increasing the sensitivity of a biomarker or diagnostic test that distinguishes between bacterial and viral infections may be desired, even though specificity may be reduced.

Whilst reducing the present invention to practice, the present inventors studied the gene expression profiles of blood leukocytes obtained from patients with acute infections. The results indicate there is a differential response of the immune system to bacterial and viral infections, which can potentially be used to classify patients within the first few days of symptom onset. Such tests would be particularly suitable in an emergency department setting.

The present inventors sought RNA markers that have a sufficiently high expression level so as not to affect the signal-to-noise ratio when measured as part of a diagnostic test. Next, they tested whether particular combinations of RNAs from this list would, when used in combination, be capable of classifying subjects as having a viral or bacterial infection with a very high accuracy.

The present inventors uncovered one such combination—CD177 RNA and IFI44L RNA which answered these requirements. Not only could this combination accurately distinguish between bacterial and viral infections, but also between bacterial and non-infectious subjects, bacterial and healthy subjects, viral and non-infectious subjects and viral and healthy subjects.

By combining this pair of RNA markers with additional RNA markers (IFIT1 RNA, MMP9 RNA and PI3 RNA), the present inventors came up with a 5 marker signature which shows sufficient accuracy that it can be used as a valuable tool in the clinical setting.

Whilst subsequently corroborating these results, the present inventors uncovered additional RNA markers that could serve as determinants for distinguishing between bacterial and viral infections (Table 3).

The present inventors also uncovered additional RNA determinants that can be used to determine the severity of infectious diseases (Tables 5-7). Pairs (Tables 8, 11 and 14) and triplets (Tables 9, 12 and 15) of these determinants were uncovered that provide very high accuracy of diagnosis.

Thus, according to a first aspect of the invention there is provided a method of ruling in a viral infection in a test subject comprising:
(a) measuring the amount of CD177 RNA and the amount of IFI44L RNA in a sample obtained from the subject;
(b) generating a score based on the amount of CD177 RNA and the amount of IFI44L RNA, wherein the score is an increasing function of the amount of CD177 RNA and a decreasing function of the amount of IFI44L RNA, wherein when the score is below a predetermined level a viral infection is ruled in, wherein the predetermined level is based on the amount of CD177 RNA and the amount of IFI44L RNA in bacterial subjects.

According to another aspect, there is provided a method of ruling in a viral infection in a test subject comprising:
(a) measuring the amount of CD177 RNA and the amount of IFI44L RNA in a sample obtained from the subject;
(b) generating a score based on the amount of CD177 RNA and the amount of IFI44L RNA, wherein the score is an increasing function of the amount of CD177 RNA and an increasing function of the amount of IFI44L RNA, wherein when the score is above a predetermined level a viral infection is ruled in, wherein the predetermined level is based on the amount of the amount of CD177 RNA and the amount of IFI44L RNA in non-infectious subjects.

According to still another aspect of the present invention, there is provided a method of ruling in a bacterial infection in a test subject comprising:
(a) measuring the amount of CD177 RNA and the amount of IFI44L RNA in a sample obtained from the subject;
(b) generating a score based on the amount of CD177RNA and the amount of IFI44L RNA, wherein the score is an increasing function of the amount of CD177 RNA and a decreasing function of the amount of IFI44L RNA, wherein when the score is above a predetermined level a bacterial infection is ruled in, wherein the predetermined level is based on the amount of CD177 RNA and the amount of IFI44L RNA in viral subjects.

According to still another aspect of the present invention there is provided a method of diagnosing the severity of a viral infection of a test subject, comprising measuring the expression level of a first RNA set forth in Table 5 and the level of a second RNA set forth in Table 6 in a sample of the subject, wherein said levels are indicative of the severity of the viral infection.

According to still another aspect of the present invention there is provided a method of diagnosing the severity of a test subject with an infectious disease, comprising measuring the expression level of pairs of RNAs set forth in Table 8 or triplets of RNAs set forth in Table 9 in a sample of the subject, wherein said levels are indicative of the severity of the infectious disease.

According to still another aspect of the present invention there is provided a method of diagnosing the severity of a viral infection of a test subject, comprising measuring the expression level of pairs of RNAs set forth in Table 11 or triplets of RNAs set forth in Table 12 in a sample of the subject, wherein said levels are indicative of the severity of the viral infection.

According to still another aspect of the present invention there is provided a method of diagnosing the severity of a bacterial disease of a test subject, comprising measuring the expression level of pairs of RNAs set forth in Table 14 or triplets of RNAs set forth in Table 15 in a sample of the subject, wherein said levels are indicative of the severity of the bacterial infection.

A “test subject” in the context of the present invention may be a mammal (e.g. human, dog, cat, horse, cow, sheep, pig or goat). According to another embodiment, the subject is a bird (e.g. chicken, turkey, duck or goose). According to a particular embodiment, the subject is a human. The subject may be male or female. The subject may be an adult (e.g. older than 18, 21, or 22 years or a child (e.g. younger than 18, 21 or 22 years). In another embodiment, the subject is an adolescent (between 12 and 21 years), an infant (29 days to less than 2 years of age) or a neonate (birth through the first 28 days of life).

The subject of these aspects of the present invention may have symptoms of an infection.

Exemplary symptoms include, but are not limited to fever, headache, cough, runny nose, chills, muscle aches, loss of taste and/or loss of smell.

According to a particular embodiment, measuring the RNA determinants described herein above is carried out no more than 24 hours following the start of symptoms, no more than 36 hours following the start of symptoms, no more than 48 hours following the start of symptoms, no more than 72 hours following the start of symptoms, no more than 96 hours following the start of symptoms, or no more than 1 week following the start of symptoms.

According to another embodiment, the subject is asymptomatic.

It will be appreciated, whether symptomatic or asymptomatic, the subject may or may not be contagious.

In one embodiment, the subject does not have a chronic non-infectious disease such as cancer, cardiac disease, a chronic immune disease or a chronic inflammatory disorder.

In one embodiment, the subject is hospitalized.

In another embodiment, the subject is non-hospitalized.

Exemplary viral diseases which may be ruled in according to the methods described herein are summarized in Table 1.

TABLE 1
Diseases

gastroenteritis
keratoconjunctivitis
pharyngitis
croup
pharyngoconjunctival fever
pneumonia
cystitis
Hand, foot and mouth disease
pleurodynia
aseptic meningitis
pericarditis
myocarditis
infectious mononucleosis
Burkitt's lymphoma
Hodgkin's lymphoma
nasopharyngeal carcinoma
acute hepatitis
chronic hepatitis
hepatic cirrhosis
hepatocellular carcinoma
herpes labialis, cold sores - can recur by latency
gingivostomatitis in children
tonsillitis & pharyngitis in adults
keratoconjunctivitis
Aseptic meningitis
infectious mononucleosis
Cytomegalic inclusion disease
Kaposi sarcoma
multicentric Castleman disease
primary effusion lymphoma
AIDS
influenza
(Reye syndrome)
measles
postinfectious encephalomyelitis
mumps
hyperplastic epithelial lesions (common, flat, plantar and anogenital
warts, laryngeal papillomas, epidermodysplasia verruciformis
Malignancies for some species (cervical carcinoma, squamous cell
carcinomas)
croup
pneumonia
bronchiolitis
common cold[
poliomyelitis
rabies (fatal encephalitis)
congenital rubella
German measles
chickenpox
herpes zoster
Congenital varicella syndrome

According to a specific embodiment, the viral disease is COVID-19.

Exemplary viruses that cause disease include those set forth in Table 2, herein below.

TABLE 2

| Family | Baltimore group | Important species | envelopment |
|---|---|---|---|
| Adenoviridae | Group I | Adenovirus | non- |
| | (dsDNA) | | enveloped |
| Herpesviridae | Group I | Herpes simplex, type 1, Herpes simplex | enveloped |
| | (dsDNA) | type 2, Varicella-zoster virus, Epstein- | |
| | | Barr virus, Human cytomegalovirus, | |
| | | Human herpesvirus, type 8 | |
| Papillomaviridae | Group I | Human papillomavirus | non- |
| | (dsDNA) | | enveloped |
| Polyomaviridae | Group I | BK virus, JC virus | non- |
| | (dsDNA) | | enveloped |
| Poxviridae | Group I | Smallpox | enveloped |
| | (dsDNA) | | |
| Hepadnaviridae | Group VII | Hepatitis B virus | enveloped |
| | (dsDNA-RT) | | |
| Parvoviridae | Group II | Parvovirus B19 | non- |
| | (ssDNA) | | enveloped |
| Astroviridae | Group IV | Human astrovirus | non- |
| | (positive- | | enveloped |
| | sense ssRNA) | | |
| Caliciviridae | Group IV | Norwalk virus | non- |
| | (positive- | | enveloped |
| | sense ssRNA) | | |
| Picornaviridae | Group IV | coxsackievirus, hepatitis A virus, | non- |
| | (positive- | poliovirus, rhinovirus | enveloped |
| | sense ssRNA) | | |
| Coronaviridae | Group IV | Severe acute respiratory syndrome | enveloped |
| | (positive- | virus | |
| | sense ssRNA) | | |
| Flaviviridae | Group IV | Hepatitis C virus, yellow fever virus, | enveloped |
| | (positive- | dengue virus, West Nile virus, TBE | |
| | sense ssRNA) | virus | |
| Togaviridae | Group IV | Rubella virus | enveloped |
| | (positive- | | |
| | sense ssRNA) | | |
| Hepeviridae | Group IV | Hepatitis E virus | non- |
| | (positive- | | enveloped |
| | sense ssRNA) | | |
| Retroviridae | Group VI | Human immunodeficiency virus (HIV) | enveloped |
| | (ssRNA-RT) | | |
| Orthomyxoviridae | Group V | Influenza virus | enveloped |
| | (negative- | | |
| | sense ssRNA) | | |
| Arenaviridae | Group V | Lassa virus | enveloped |
| | (negative- | | |
| | sense ssRNA) | | |
| Bunyaviridae | Group V | Crimean-Congo hemorrhagic fever | enveloped |
| | (negative- | virus, Hantaan virus | |
| | sense ssRNA) | | |
| Filoviridae | Group V | Ebola virus, Marburg virus | enveloped |
| | (negative- | | |
| | sense ssRNA) | | |
| Paramyxoviridae | Group V | Measles virus, Mumps virus, | enveloped |
| | (negative- | Parainfluenza virus, Respiratory | |
| | sense ssRNA | syncytial virus, | |
| Rhabdoviridae | Group V | Rabies virus | enveloped |
| | (negative- | | |
| | sense ssRNA) | | |
| Unassigned | Group V | Hepatitis D | enveloped |
| | (negative- | | |
| | sense ssRNA | | |
| Reoviridae | Group III | Rotavirus, Orbivirus, Coltivirus, | non- |
| | (dsRNA) | Banna virus | enveloped |

According to a specific embodiment, the virus is SARS-CoV-2.

According to another specific embodiment, the virus is Human metapneumovirus, Bocavirus or Enterovirus.

According to another specific embodiment, the virus is RSV, Flu A, Flu B, HCoV or SARS-Cov-2.

Examples of coronaviruses include: human coronavirus 229E, human coronavirus OC43, SARS-CoV, HCoV NL63, HKU1, MERS-CoV and SARS-CoV-2.

According to a particular embodiment, the coronavirus is SARS-CoV-2.

Bacterial infections which may be ruled in according to embodiments of the invention may be the result of gram-positive, gram-negative bacteria or atypical bacteria.

The term “Gram-positive bacteria” refers to bacteria that are stained dark blue by Gram staining. Gram-positive organisms are able to retain the crystal violet stain because of the high amount of peptidoglycan in the cell wall.

The term “Gram-negative bacteria” refers to bacteria that do not retain the crystal violet dye in the Gram staining protocol.

The term “Atypical bacteria” are bacteria that do not fall into one of the classical “Gram” groups. They are usually, though not always, intracellular bacterial pathogens. They include, without limitations, Mycoplasmas spp., Legionella spp. Rickettsia spp., and Chlamydia spp. The bacterial or viral infection may be an acute or chronic infection.

A chronic infection is an infection that develops slowly and lasts a long time. Viruses that may cause a chronic infection include Hepatitis C and HIV. One difference between acute and chronic infection is that during acute infection the immune system often produces IgM+ antibodies against the infectious agent, whereas the chronic phase of the infection is usually characteristic of IgM−/IgG+ antibodies. In addition, acute infections cause immune mediated necrotic processes while chronic infections often cause inflammatory mediated fibrotic processes and scaring (e.g. Hepatitis C in the liver). Thus, acute and chronic infections may elicit different underlying immunological mechanisms.

According to a particular embodiment, the infection that is ruled in is an acute infection.

“Measuring” or “measurement,” or alternatively “detecting” or “detection,” means assessing the presence, absence, quantity or amount (which can be an effective amount) of the determinant within a clinical or subject-derived sample, including the derivation of qualitative or quantitative concentration levels of such determinants.

A “sample” in the context of the present invention is a biological sample isolated from a subject and can include, by way of example and not limitation, whole blood, serum, plasma, saliva, mucus, breath, urine, CSF, sputum, sweat, stool, hair, seminal fluid, biopsy, rhinorrhea, tissue biopsy, cytological sample, platelets, reticulocytes, leukocytes, epithelial cells, or whole blood cells.

For measuring the amounts of (or the level of) RNA determinants, preferably the sample is a blood sample comprising white blood cells such as lymphocytes, monocytes and granulocytes (which is depleted of red blood cells). In one embodiment, the sample is not a serum sample.

Methods of depleting red blood cells are known in the art and include for example hemolysis, centrifugation, sedimentation, filtration or combinations thereof.

The sample may comprise RNA from a heterogeneous population of cells or from a single population of cells. The RNA may comprise total RNA, mRNA, mitochondrial RNA, chloroplast RNA, DNA-RNA hybrids, viral RNA, cell free RNA, and mixtures thereof. In one embodiment, the RNA sample is devoid of DNA.

The sample may be fresh or frozen.

Isolation, extraction or derivation of RNA may be carried out by any suitable method. Isolating RNA from a biological sample generally includes treating a biological sample in such a manner that the RNA present in the sample is extracted and made available for analysis. Any isolation method that results in extracted RNA may be used in the practice of the present invention. It will be understood that the particular method used to extract RNA will depend on the nature of the source.

Methods of RNA extraction are well-known in the art and further described herein under.

Phenol based extraction methods: These single-step RNA isolation methods based on Guanidine isothiocyanate (GITC)/phenol/chloroform extraction require much less time than traditional methods (e.g. CsCl₂ ultracentrifugation). Many commercial reagents (e.g. Trizol, RNAzol, RNAWIZ) are based on this principle. The entire procedure can be completed within an hour to produce high yields of total RNA.

Silica gel—based purification methods: RNeasy is a purification kit marketed by Qiagen. It uses a silica gel-based membrane in a spin-column to selectively bind RNA larger than 200 bases. The method is quick and does not involve the use of phenol.

Oligo-dT based affinity purification of mRNA: Due to the low abundance of mRNA in the total pool of cellular RNA, reducing the amount of rRNA and tRNA in a total RNA preparation greatly increases the relative amount of mRNA. The use of oligo-dT affinity chromatography to selectively enrich poly (A)+ RNA has been practiced for over 20 years. The result of the preparation is an enriched mRNA population that has minimal rRNA or other small RNA contamination. mRNA enrichment is essential for construction of cDNA libraries and other applications where intact mRNA is highly desirable. The original method utilized oligo-dT conjugated resin column chromatography and can be time consuming. Recently more convenient formats such as spin-column and magnetic bead based reagent kits have become available.

The sample may also be processed prior to carrying out the diagnostic methods of the present invention. Processing of the sample may involve one or more of: filtration, distillation, centrifugation, extraction, concentration, dilution, purification, inactivation of interfering components, addition of reagents, and the like.

After obtaining the RNA sample, cDNA may be generated therefrom. For synthesis of cDNA, template mRNA may be obtained directly from lysed cells or may be purified from a total RNA or mRNA sample. The total RNA sample may be subjected to a force to encourage shearing of the RNA molecules such that the average size of each of the RNA molecules is between 100-300 nucleotides, e.g. about 200 nucleotides. To separate the heterogeneous population of mRNA from the majority of the RNA found in the cell, various technologies may be used which are based on the use of oligo(dT) oligonucleotides attached to a solid support. Examples of such oligo(dT) oligonucleotides include: oligo(dT) cellulose/spin columns, oligo(dT)/magnetic beads, and oligo(dT) oligonucleotide coated plates.

Generation of single stranded DNA from RNA requires synthesis of an intermediate RNA-DNA hybrid. For this, a primer is required that hybridizes to the 3′ end of the RNA. Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about minutes.

According to a specific embodiment, the primer comprises a polydT oligonucleotide sequence.

Preferably the polydT sequence comprises at least 5 nucleotides. According to another is between about 5 to 50 nucleotides, more preferably between about 5-25 nucleotides, and even more preferably between about 12 to 14 nucleotides.

Following annealing of the primer (e.g. polydT primer) to the RNA sample, an RNA-DNA hybrid is synthesized by reverse transcription using an RNA-dependent DNA polymerase. Suitable RNA-dependent DNA polymerases for use in the methods and compositions of the invention include reverse transcriptases (RTs). Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules.

Additional components required in a reverse transcription reaction include dNTPS (dATP, dCTP, dGTP and dTTP) and optionally a reducing agent such as Dithiothreitol (DTT) and MnCl₂.

Methods of analyzing the amount of RNA are known in the art and are summarized infra:

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls. Isothermal amplification is also contemplated.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, CA).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

Oligonucleotide microarray—In this method oligonucleotide probes capable of specifically hybridizing with the polynucleotides of some embodiments of the invention are attached to a solid surface (e.g., a glass wafer). Each oligonucleotide probe is of approximately 20-25 nucleic acids in length. To detect the expression pattern of the polynucleotides of some embodiments of the invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, MD, USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara CA). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, CA) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

RNA sequencing: Methods for RNA sequence determination are generally known to the person skilled in the art. Preferred sequencing methods are next generation sequencing methods or parallel high throughput sequencing methods. An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

It will be appreciated that in order to analyze the amount of an RNA marker, oligonucleotides may be used that are capable of hybridizing thereto or to cDNA generated therefrom. According to one embodiment a single oligonucleotide is used to determine the presence of a particular RNA marker, at least two oligonucleotides are used to determine the presence of a particular RNA marker, at least three oligonucleotides are used to determine the presence of a particular RNA marker, at least four oligonucleotides are used to determine the presence of a particular RNA marker, at least five or more oligonucleotides are used to determine the presence of a particular RNA marker.

When more than one oligonucleotide is used, the sequence of the oligonucleotides may be selected such that they hybridize to the same exon of the RNA marker or different exons of the RNA marker. In one embodiment, at least one of the oligonucleotides hybridizes to the 3′ exon of the RNA marker. In another embodiment, at least one of the oligonucleotides hybridizes to the 5′ exon of the RNA marker.

In one embodiment, the method of this aspect of the present invention is carried out using an isolated oligonucleotide which hybridizes to the RNA or cDNA of any of the RNA markers disclosed herein by complementary base-pairing in a sequence specific manner, and discriminates the determinant sequence from other nucleic acid sequence in the sample. Oligonucleotides (e.g. DNA or RNA oligonucleotides) typically comprises a region of complementary nucleotide sequence that hybridizes under stringent conditions to at least about 8, 10, 13, 16, 18, 20, 22, 25, 30, 40, 50, 55, 60, 70, 80, 90, 100, 120 (or any other number in-between) or more consecutive nucleotides in a target nucleic acid molecule. Depending on the particular assay, the consecutive nucleotides include the determinant nucleic acid sequence.

The term “isolated”, as used herein in reference to an oligonucleotide, means an oligonucleotide, which by virtue of its origin or manipulation, is separated from at least some of the components with which it is naturally associated or with which it is associated when initially obtained. By “isolated”, it is alternatively or additionally meant that the oligonucleotide of interest is produced or synthesized by the hand of man.

In order to identify an oligonucleotide specific for any of the RNA markers disclosed herein, the gene/transcript of interest is typically examined using a computer algorithm which starts at the 5′ or at the 3′ end of the nucleotide sequence. Typical algorithms will then identify oligonucleotides of defined length that are unique to the gene, have a GC content within a range suitable for hybridization, lack predicted secondary structure that may interfere with hybridization, and/or possess other desired characteristics or that lack other undesired characteristics.

Following identification of the oligonucleotide it may be tested for specificity towards the determinant under wet or dry conditions. Thus, for example, in the case where the oligonucleotide is a primer, the primer may be tested for its ability to amplify a sequence of the determinant using PCR to generate a detectable product and for its non ability to amplify other determinants in the sample. The products of the PCR reaction may be analyzed on a gel and verified according to presence and/or size.

Additionally, or alternatively, the sequence of the oligonucleotide may be analyzed by computer analysis to see if it is homologous (or is capable of hybridizing to) other known sequences. A BLAST 2.2.10 (Basic Local Alignment Search Tool) analysis may be performed on the chosen oligonucleotide (worldwidewebdotncbidotnlmdotnihdotgov/blast/). The BLAST program finds regions of local similarity between sequences. It compares nucleotide or protein sequences to sequence databases and calculates the statistical significance of matches thereby providing valuable information about the possible identity and integrity of the ‘query’ sequences.

According to one embodiment, the oligonucleotide is a probe. As used herein, the term “probe” refers to an oligonucleotide which hybridizes to the determinant specific nucleic acid sequence to provide a detectable signal under experimental conditions and which does not hybridize to additional determinant sequences to provide a detectable signal under identical experimental conditions.

The probes of this embodiment of this aspect of the present invention may be, for example, affixed to a solid support (e.g., arrays or beads).

Solid supports are solid-state substrates or supports onto which the nucleic acid molecules of the present invention may be associated. The nucleic acids may be associated directly or indirectly. Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

In one embodiment, the solid support is an array which comprises a plurality of nucleic acids which hybridize to RNA markers of the present invention immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

According to particular embodiments, the array does not comprise nucleic acids that specifically bind to more than 50 RNA markers, more than 40 RNA markers, 30 RNA markers, 20 RNA markers, 15 RNA markers, 10 RNA markers, 5 RNA markers or even 3 RNA markers.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

According to another embodiment, the oligonucleotide is a primer of a primer pair. As used herein, the term “primer” refers to an oligonucleotide which acts as a point of initiation of a template-directed synthesis using methods such as PCR (polymerase chain reaction) or LCR (ligase chain reaction) under appropriate conditions (e.g., in the presence of four different nucleotide triphosphates and a polymerization agent, such as DNA polymerase, RNA polymerase or reverse-transcriptase, DNA ligase, etc, in an appropriate buffer solution containing any necessary co-factors and at suitable temperature(s)). Such a template directed synthesis is also called “primer extension”. For example, a primer pair may be designed to amplify a region of DNA using PCR. Such a pair will include a “forward primer” and a “reverse primer” that hybridize to complementary strands of a DNA molecule and that delimit a region to be synthesized/amplified. A primer of this aspect of the present invention is capable of amplifying, together with its pair (e.g. by PCR) a determinant specific nucleic acid sequence to provide a detectable signal under experimental conditions and which does not amplify other determinant nucleic acid sequence to provide a detectable signal under identical experimental conditions.

According to additional embodiments, the oligonucleotide is about 8, 9, 10, 11, 12, 13, 14, 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. It will be appreciated that when attached to a solid support, the probe may be of about 30-70, 75, 80, 90, 100, or more nucleotides in length.

The oligonucleotide of this aspect of the present invention need not reflect the exact sequence of the RNA marker nucleic acid sequence (i.e. need not be fully complementary), but must be sufficiently complementary to hybridize with the determinant nucleic acid sequence under the particular experimental conditions. Accordingly, the sequence of the oligonucleotide typically has at least 70% homology, preferably at least 80%, 90%, 95%, 97%, 99% or 100% homology, for example over a region of at least 13 or more contiguous nucleotides with the target determinant nucleic acid sequence. The conditions are selected such that hybridization of the oligonucleotide to the determinant nucleic acid sequence is favored and hybridization to other determinant nucleic acid sequences is minimized.

By way of example, hybridization of short nucleic acids (below 200 bp in length, e.g. 13-50 bp in length) can be effected by the following hybridization protocols depending on the desired stringency; (i) hybridization solution of 6×SSC and 1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tm, final wash solution of 3 M TMACl, 0.01 M sodium phosphate (pH 6.8), 1 mM EDTA (pH 7.6), 0.5% SDS at 1-1.5° C. below the Tm (stringent hybridization conditions) (ii) hybridization solution of 6×SSC and 0.1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACl, 0.01 M sodium phosphate (pH 6.8), 1 mM EDTA (pH 7.6), 0.5% SDS at 1-1.5° C. below the Tm, final wash solution of 6×SSC, and final wash at 22° C. (stringent to moderate hybridization conditions); and (iii) hybridization solution of 6×SSC and 1% SDS or 3 M TMACl, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature at 2.5-3° C. below the Tm and final wash solution of 6×SSC at 22° C. (moderate hybridization solution).

Oligonucleotides of the invention may be prepared by any of a variety of methods (see, for example, J. Sambrook et al., “Molecular Cloning: A Laboratory Manual”, 1989, 2.sup.nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5.sup.th Ed., John Wiley & Sons: Secaucus, N.J.). For example, oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066.

For example, oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of the oligonucleotides of the invention, where necessary or desirable, may be carried out by any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

The sequence of oligonucleotides can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), mass spectrometry following a combination of alkaline phosphatase and exonuclease digestions (H. Wu and H. Aboleneen, Anal. Biochem., 2001, 290: 347-352), and the like.

As already mentioned above, modified oligonucleotides may be prepared using any of several means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc), or charged linkages (e.g., phosphorothioates, phosphorodithioates, etc). Oligonucleotides may contain one or more additional covalently linked moieties, such as, for example, proteins (e.g., nucleases, toxins, antibodies, signal peptides, poly-L-lysine, etc), intercalators (e.g., acridine, psoralen, etc), chelators (e.g., metals, radioactive metals, iron, oxidative metals, etc), and alkylators. The oligonucleotide may also be derivatized by formation of a methyl or ethyl phosphotriester or an alkyl phosphoramidate linkage. Furthermore, the oligonucleotide sequences of the present invention may also be modified with a label.

In certain embodiments, the detection probes or amplification primers or both probes and primers are labeled with a detectable agent or moiety before being used in amplification/detection assays. In certain embodiments, the detection probes are labeled with a detectable agent. Preferably, a detectable agent is selected such that it generates a signal which can be measured and whose intensity is related (e.g., proportional) to the amount of amplification products in the sample being analyzed.

The association between the oligonucleotide and detectable agent can be covalent or non-covalent. Labeled detection probes can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Methods for labeling nucleic acid molecules are well-known in the art. For a review of labeling protocols, label detection techniques, and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachments of fluorescent dyes (L. M. Smith et al., Nucl. Acids Res., 1985, 13: 2399-2412) or of enzymes (B. A. Connoly and O. Rider, Nucl. Acids. Res., 1985, 13: 4485-4502); chemical modifications of nucleic acid molecules making them detectable immunochemically or by other affinity reactions (T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of mono-reactive cisplatin derivatives with the N7 position of guanine moieties in DNA (R. J. Heetebrij et al., Cytogenet. Cell. Genet. 1999, 87: 47-52), psoralen-biotin, which intercalates into nucleic acids and upon UV irradiation becomes covalently bonded to the nucleotide bases (C. Levenson et al., Methods Enzymol. 1990, 184: 577-583; and C. Pfannschmidt et al., Nucleic Acids Res. 1996, 24: 1702-1709), photoreactive azido derivatives (C. Neves et al., Bioconjugate Chem. 2000, 11: 51-55), and DNA alkylating agents (M. G. Sebestyen et al., Nat. Biotechnol. 1998, 16: 568-576).

Any of a wide variety of detectable agents can be used in the practice of the present invention. Suitable detectable agents include, but are not limited to, various ligands, radionuclides (such as, for example, ³²P, ³⁵S, ³H, ¹⁴C, ¹²⁵I, ¹³¹I, and the like); fluorescent dyes (for specific exemplary fluorescent dyes, see below); chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, i.e., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens and proteins for which antisera or monoclonal antibodies are available.

In certain embodiments, the detection probes are fluorescently labeled. Numerous known fluorescent labeling moieties of a wide variety of chemical structures and physical characteristics are suitable for use in the practice of this invention. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxy-fluorescein, 6 carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for linking or incorporating fluorescent dyes to nucleic acid molecules see, for example, “The Handbook of Fluorescent Probes and Research Products”, 9th Ed., Molecular Probes, Inc., Eugene, Oreg. Fluorescent dyes as well as labeling kits are commercially available from, for example, Amersham Biosciences, Inc. (Piscataway, N.J.), Molecular Probes Inc. (Eugene, Oreg.), and New England Biolabs Inc. (Berverly, Mass.).

As mentioned, identification of the RNA marker may be carried out using an amplification reaction.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

A typical amplification reaction is carried out by contacting a forward and reverse primer (a primer pair) to the sample DNA together with any additional amplification reaction reagents under conditions which allow amplification of the target sequence.

The terms “forward primer” and “forward amplification primer” are used herein interchangeably, and refer to a primer that hybridizes (or anneals) to the target (template strand). The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphospate, deoxyguanosine triphosphate, deoxycytidine triphosphate and thymidine triphosphate. Amplification reaction reagents may readily be selected by one skilled in the art depending on the amplification method used.

According to this aspect of the present invention, the amplifying may be effected using techniques such as polymerase chain reaction (PCR), which includes, but is not limited to Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification, Multiplex-PCR, Nested PCR, Overlap-extension PCR, Quantitative PCR (Q-PCR), Reverse Transcription PCR (RT-PCR), Solid Phase PCR: encompasses multiple meanings, including Polony Amplification (where PCR colonies are derived in a gel matrix, for example), Bridge PCR (primers are covalently linked to a solid-support surface), conventional Solid Phase PCR (where Asymmetric PCR is applied in the presence of solid support bearing primer with sequence matching one of the aqueous primers) and Enhanced Solid Phase PCR (where conventional Solid Phase PCR can be improved by employing high Tm and nested solid support primer with optional application of a thermal ‘step’ to favour solid support priming), Thermal asymmetric interlaced PCR (TAIL-PCR), Touchdown PCR (Step-down PCR), PAN-AC and Universal Fast Walking.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stearothermophilus (Bio-Rad), or Thermococcus litoralis (“Vent” polymerase, New England Biolabs). RNA target sequences may be amplified by reverse transcribing the mRNA into cDNA, and then performing PCR (RT-PCR), as described above. Alternatively, a single enzyme may be used for both steps as described in U.S. Pat. No. 5,322,770.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually from about 93° C. to about 96° C. for a period of time ranging from about 3 to about 120 seconds, usually from about 5 to about 30 seconds.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 50° C. to about ° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

Following annealing of primer to template DNA or during annealing of primer to template DNA, the reaction mixture is subjected to conditions sufficient to provide for polymerization of nucleotides to the primer's end in a such manner that the primer is extended in a 5′ to 3′ direction using the DNA to which it is hybridized as a template, (i.e., conditions sufficient for enzymatic production of primer extension product). To achieve primer extension conditions, the temperature of the reaction mixture is typically raised to a temperature ranging from about 65° C. to about 75° C., usually from about 67° C. to about 73° C., and maintained at that temperature for a period of time ranging from about 15 seconds to about 20 minutes, usually from about 30 seconds to about 5 minutes.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

Amplification products obtained using primers of the present invention may be detected using agarose gel electrophoresis and visualization by ethidium bromide staining and exposure to ultraviolet (UV) light or by sequence analysis of the amplification product.

According to one embodiment, the amplification and quantification of the amplification product may be effected in real-time (qRT-PCR). Typically, QRT-PCR methods use double stranded DNA detecting molecules to measure the amount of amplified product in real time.

As used herein the phrase “double stranded DNA detecting molecule” refers to a double stranded DNA interacting molecule that produces a quantifiable signal (e.g., fluorescent signal). For example such a double stranded DNA detecting molecule can be a fluorescent dye that (1) interacts with a fragment of DNA or an amplicon and (2) emits at a different wavelength in the presence of an amplicon in duplex formation than in the presence of the amplicon in separation. A double stranded DNA detecting molecule can be a double stranded DNA intercalating detecting molecule or a primer-based double stranded DNA detecting molecule.

A double stranded DNA intercalating detecting molecule is not covalently linked to a primer, an amplicon or a nucleic acid template. The detecting molecule increases its emission in the presence of double stranded DNA and decreases its emission when duplex DNA unwinds. Examples include, but are not limited to, ethidium bromide, YO-PRO-1, Hoechst 33258, SYBR Gold, and SYBR Green I. Ethidium bromide is a fluorescent chemical that intercalates between base pairs in a double stranded DNA fragment and is commonly used to detect DNA following gel electrophoresis. When excited by ultraviolet light between 254 nm and 366 nm, it emits fluorescent light at 590 nm. The DNA-ethidium bromide complex produces about 50 times more fluorescence than ethidium bromide in the presence of single stranded DNA. SYBR Green I is excited at 497 nm and emits at 520 nm. The fluorescence intensity of SYBR Green I increases over 100 fold upon binding to double stranded DNA against single stranded DNA. An alternative to SYBR Green I is SYBR Gold introduced by Molecular Probes Inc. Similar to SYBR Green I, the fluorescence emission of SYBR Gold enhances in the presence of DNA in duplex and decreases when double stranded DNA unwinds. However, SYBR Gold's excitation peak is at 495 nm and the emission peak is at 537 nm. SYBR Gold reportedly appears more stable than SYBR Green I. Hoechst 33258 is a known bisbenzimide double stranded DNA detecting molecule that binds to the AT rich regions of DNA in duplex. Hoechst 33258 excites at 350 nm and emits at 450 nm. YO-PRO-1, exciting at 450 nm and emitting at 550 nm, has been reported to be a double stranded DNA specific detecting molecule. In a particular embodiment of the present invention, the double stranded DNA detecting molecule is SYBR Green I.

A primer-based double stranded DNA detecting molecule is covalently linked to a primer and either increases or decreases fluorescence emission when amplicons form a duplex structure. Increased fluorescence emission is observed when a primer-based double stranded DNA detecting molecule is attached close to the 3′ end of a primer and the primer terminal base is either dG or dC.

The detecting molecule is quenched in the proximity of terminal dC-dG and dG-dC base pairs and dequenched as a result of duplex formation of the amplicon when the detecting molecule is located internally at least 6 nucleotides away from the ends of the primer. The dequenching results in a substantial increase in fluorescence emission. Examples of these type of detecting molecules include but are not limited to fluorescein (exciting at 488 nm and emitting at 530 nm), FAM (exciting at 494 nm and emitting at 518 nm), JOE (exciting at 527 and emitting at 548), HEX (exciting at 535 nm and emitting at 556 nm), TET (exciting at 521 nm and emitting at 536 nm), Alexa Fluor 594 (exciting at 590 nm and emitting at 615 nm), ROX (exciting at 575 nm and emitting at 602 nm), and TAMRA (exciting at 555 nm and emitting at 580 nm). In contrast, some primer-based double stranded DNA detecting molecules decrease their emission in the presence of double stranded DNA against single stranded DNA. Examples include, but are not limited to, rhodamine, and BODIPY-FI (exciting at 504 nm and emitting at 513 nm). These detecting molecules are usually covalently conjugated to a primer at the 5′ terminal dC or dG and emit less fluorescence when amplicons are in duplex. It is believed that the decrease of fluorescence upon the formation of duplex is due to the quenching of guanosine in the complementary strand in close proximity to the detecting molecule or the quenching of the terminal dC-dG base pairs.

According to one embodiment, the primer-based double stranded DNA detecting molecule is a 5′ nuclease probe. Such probes incorporate a fluorescent reporter molecule at either the 5′ or 3′ end of an oligonucleotide and a quencher at the opposite end. The first step of the amplification process involves heating to denature the double stranded DNA target molecule into a single stranded DNA. During the second step, a forward primer anneals to the target strand of the DNA and is extended by Taq polymerase. A reverse primer and a 5′ nuclease probe then anneal to this newly replicated strand.

In this embodiment, at least one of the primer pairs or 5′ nuclease probe should hybridize with a unique determinant sequence. The polymerase extends and cleaves the probe from the target strand. Upon cleavage, the reporter is no longer quenched by its proximity to the quencher and fluorescence is released. Each replication will result in the cleavage of a probe. As a result, the fluorescent signal will increase proportionally to the amount of amplification product.

According to one aspect, in order to determine the type of infection, both CD177 RNA and IFI44L RNA are measured.

In one embodiment, the diagnosing stage is carried out by generating a score based on the amount of both CD177 RNA and IFI44L RNA.

The present inventors have shown that compared to subjects with a known bacterial infection, CD177 is decreased and IFI44L is increased in virally infected subjects.

In one embodiment, the score is an increasing function of the amount of CD177 RNA and a decreasing function of the amount of IFI44L RNA. In this case, when the score is below a predetermined level a viral infection is ruled in, the predetermined level being based on the amount of both CD177 RNA and IFI44L RNA in bacterial subjects.

The score may be a monotonically increasing function of the amount of CD177 RNA and a monotonically decreasing function of the amount of IFI44L RNA. In one embodiment, the function is linear.

In another embodiment, the score may be a decreasing function of the amount of CD177 RNA and an increasing function of the amount of IFI44L RNA. In this case, when the score is above a predetermined level a viral infection is ruled in, the predetermined level being based on the amount of both CD177 RNA and IFI44L RNA in bacterial subjects.

The score may be a monotonically decreasing function of CD177 RNA and a monotonically increasing function of the amount of IFI44L RNA. In one embodiment, the function is linear. In one embodiment, the score is based on the ratio of CD177:IFI44L, when the ratio is below a predetermined level a viral infection is ruled in, the predetermined level being based on the amount of CD177 RNA and IFI44L RNA in bacterial subjects.

In still another embodiment, the score is based on the ratio of IFI44L:CD177, when the ratio is above a predetermined level a viral infection is ruled in, the predetermined level being based on the amount of CD177 RNA and IFI44L RNA in bacterial subjects.

In still another embodiment, when the level of CD177 RNA is below a predetermined level (e.g. below the level that is present in a sample derived from a subject known to be infected with a bacterial infection), and when the level of IFI44L RNA is above a predetermined level (e.g. above the level that is present in a sample derived from a non-infectious subject, e.g. healthy subject; or above the level that is present in a bacterially infected subject) it is indicative that the subject has a viral infection (i.e. a viral infection may be ruled in).

The present inventors have shown that compared to subjects with a known viral infection, CD177 is increased and IFI44L is decreased, in bacterially infected subjects.

For this aspect, the score is an increasing function of the amount of CD177 RNA and a decreasing function of the amount of IFI44L RNA. In this case, when the score is above a predetermined level a bacterial infection is ruled in, the predetermined level being based on the amount of CD177 RNA and IFI44L RNA in viral subjects.

The score may be a monotonically increasing function of CD177 RNA and a monotonically decreasing function of the amount of IFI44L RNA. In one embodiment, the function is linear. In another embodiment, the score may be a decreasing function of the amount of CD177 RNA and an increasing function of the amount of IFI44L RNA. In this case, when the score is below a predetermined level, a bacterial infection is ruled in, the predetermined level being based on the amount of CD177 RNA and IFI44L RNA in viral subjects.

The score may be a monotonically decreasing function of the amount of CD177 RNA and a monotonically increasing function of the amount of IFI44L RNA. In one embodiment, the function is linear.

In one embodiment, the score is based on the ratio of CD177:IFI44L, when the ratio is above a predetermined level a bacterial infection is ruled in, the predetermined level being based on the amount of CD177 RNA and IFI44L in viral subjects.

In still another embodiment, the score is based on the ratio of IFI44L:CD177, when the ratio is below a predetermined level a bacterial infection is ruled in, the predetermined level being based on the amount of CD177 RNA and IFI44L RNA in viral subjects.

In still another embodiment, when the level of CD177 RNA is above a predetermined level (e.g. above the level that is present in a sample derived from a subject known to be infected with a viral infection or above the level that is present in a non-infectious subject (e.g. healthy subject), and when the level of IFI44L RNA is below a predetermined level (e.g. below the level that is present in a sample derived from a non-infectious subject, e.g. healthy subject; or below the level that is present in a virally infected subject) it is indicative that the subject has a bacterial infection (i.e. a bacterial infection may be ruled in).

The present inventors have shown that compared to non-infectious subjects (e.g. healthy subjects), CD177 is slightly increased and IFI44L is increased, in virally infected subjects. In this case, the weight of IFI44L may be higher than the weight of the CD177 in the score.

For this aspect, the score is an increasing function of the amount of CD177 RNA and an increasing function of the amount of IFI44L RNA. In this case, when the score is above a predetermined level a viral infection is ruled in, the predetermined level being based on the amount of both CD177 RNA and IFI44L RNA in non-infectious subjects (e.g. healthy subjects).

The score may be a monotonically increasing function of CD177 RNA and a monotonically increasing function of the amount of IFI44L RNA. In one embodiment, the function is linear. In another embodiment, the score may be a decreasing function of the amount of CD177 RNA and a decreasing function of the amount of IFI44L RNA. In this case, when the score is below a predetermined level, a viral infection is ruled in, the predetermined level being based on the amount of both CD177 RNA and IFI44L RNA in non-infectious subjects (e.g. healthy subjects).

The score may be a monotonically decreasing function of CD177 RNA and a monotonically decreasing function of the amount of IFI44L RNA. In one embodiment, the function is linear.

In still another embodiment, when the level of CD177 RNA is above a predetermined level (e.g. above the level that is present in a sample derived from a non-infectious subject (e.g. healthy subject), and when the level of IFI44L RNA is above a predetermined level (e.g. above the level that is present in a sample derived from a non-infectious subject, e.g. healthy subject) it is indicative that the subject has a viral infection (i.e. a viral infection may be ruled in).

The predetermined level of any of the aspects of the present invention may be a reference value derived from population studies, including without limitation, such subjects having a known infection, subject having the same or similar age range, subjects in the same or similar ethnic group, or relative to the starting sample of a subject undergoing treatment for an infection. Such reference values can be derived from statistical analyses and/or risk prediction data of populations obtained from mathematical algorithms and computed indices of infection. Reference determinant indices can also be constructed and used using algorithms and other methods of statistical and structural classification, as exemplified in Example 3, herein below.

In one embodiment of the present invention, the predetermined level is the amount (i.e. level) of (or a function of the amount of) RNA in a control sample derived from one or more subjects who do not have an infection (i.e., healthy, and or non-infectious individuals). In a further embodiment, such subjects are monitored and/or periodically retested for a diagnostically relevant period of time (“longitudinal studies”) following such test to verify continued absence of infection. Such period of time may be one day, two days, two to five days, five days, five to ten days, ten days, or ten or more days from the initial testing date for determination of the reference value. Furthermore, retrospective measurement of RNA levels in properly banked historical subject samples may be used in establishing these reference values, thus shortening the study time required.

A reference value can also comprise the amounts of RNAs derived from subjects who show an improvement as a result of treatments and/or therapies for the infection. A reference value can also comprise the amounts of RNAs derived from subjects who have confirmed infection by known techniques.

An example of a bacterially infected reference value is the mean or median concentrations of that determinant in a statistically significant number of subjects having been diagnosed as having a bacterial infection. It will be appreciated that the bacterially infected reference value may also be a function of the mean or median concentration.

An example of a virally infected reference value is the mean or median concentrations of that determinant in a statistically significant number of subjects having been diagnosed as having a viral infection. It will be appreciated that the virally infected reference value may also be a function of the mean or median concentration.

It will be appreciated that the control sample is the same sample type as the sample being analyzed.

Generating scores (i.e. construction of clinical algorithms) may be carried out using methods known in the art and are discussed in detail below.

It will be appreciated that as well as measuring CD177 RNA and IFI44L RNA, the present inventors contemplate measuring at least one of IFIT1 RNA, MMP9 RNA and PI3 RNA to determine infection type. The scores may be adjusted to take into account the amounts of at least one of these RNA.

Thus, for example the score may be adjusted based on the level of IFIT1 RNA. The present inventors have shown that the level of IFIT1 RNA is increased in virally infected subjects compared to subjects with bacterial infections or non-infectious (e.g. healthy) subjects.

In one embodiment, when the amount of CD177 RNA is below a predetermined level, IFI44L RNA is above a predetermined level and IFIT1 RNA are above a predetermined level, a viral infection may be ruled in.

In another embodiment, when the amount of CD177 RNA is above a predetermined level, the amount of IFI44L is below a predetermined level, and the amount of IFIT1 RNA is below a predetermined level, the infection is a bacterial infection.

As another example, the score may be adjusted based on the level of MMP9 RNA. The present inventors have shown that the level of MMP9 RNA is increased in subjects with bacterial infections compared to subjects with viral infections or non-infectious (e.g. healthy) subjects.

As another example, the score may be adjusted based on the level of PI3 RNA. The present inventors have shown that the level of PI3 RNA is decreased in subjects with a viral infections compared to subjects with bacterially infected subjects or non-infectious (e.g. healthy) subjects.

In still another embodiment, when the amount of CD177 RNA is below a predetermined level, the amount of IFI44L RNA is above a predetermined level, the amount of IFIT1 RNA is above a predetermined level, the amount of MMP9 RNA is below a predetermined level and the amount of PI3 RNA is below a predetermined level the infection is a viral infection.

In still another embodiment, when the amount of CD177 RNA is above a predetermined level, the amount of IFI44L RNA is below a predetermined level, the amount of IFIT1 RNA is below a predetermined level, the amount of MMP9 RNA is above a predetermined level and the amount of PI3 RNA is above a predetermined level the infection is a bacterial infection.

In addition to the RNA markers described herein above (i.e. CD177+IFI44L or CD177+IFI44L+IFIT1 or CD177+IFI44L+IFIT1+MMP9+PI3), the present inventors further contemplate measurement of at least one additional RNA marker—e.g. one that is listed in Table 3.

TABLE 3

| | 1 | 2 | 3 | 4 | 5 |
|---|---|---|---|---|---|
| | Early | B-V, | B-V, | B-NI, | B-H, |
| | response | high | high in | high in | high in |
| | to viral | in viral | bacterial | bacterial | bacterial |
| Gene | infection | infection | infection | infection | infection |
| ACSL4 | | | | x | |
| ALPL | | | | | x |
| ARG1 | | | | | x |
| BNIP2 | | | | x | |
| CA4 | | | x | | |
| EPSTI1 | | x | | | |
| GYG1 | | | | x | x |
| HERC5 | x | x | | | |
| HP | | | | | x |
| HPGD | | | | x | |
| IFI27 | | x | | | |
| IFI44 | x | x | | | |
| IFIT3 | x | x | | | |
| IFIT5 | x | | | | |
| IL18R1 | | | | x | |
| IMPA2 | | | x | | |
| ISG15 | | x | | | |
| LCN2 | | | | | x |
| LY6E | | x | | | |
| MMP8 | | | | x | |
| MS4A4A | | | | x | |
| NLRC4 | | | x | | |
| ORM1 | | | x | | |
| PGD | | | x | | |
| PGLYRP1 | | | x | | x |
| RETN | | | | | x |
| RSAD2 | | x | | | |
| S100A12 | | | | | x |
| S100P | | | x | | |
| SLPI | | | x | | |
| SMPDL3A | | | | x | |
| TSPO | | | x | | |
| VNN1 | | | | x | |
| ZDHHC19 | | | | | x |

Contemplated pairs of markers that can be added to the combination of CD177+IFIF44L include one from column 2 of Table 3 and one from column 3 of Table 3.

According to any of the aspects of the present invention, in order to distinguish between the different infection types (e.g. rule in a viral infection or rule in a bacterial infection), no more than 30 RNA markers are measured, no more than 25 RNA markers are measured, no more than 20 RNA markers are measured, no more than 15 RNA markers are measured, no more than 10 RNA markers are measured, no more than 5 RNA markers are measured, no more than 4 RNA markers are measured, no more than 3 RNA markers are measured or even no more than 2 RNA markers are measured.

According to any of the aspects of the present invention, in order to distinguish between the different infection types, no more than 30 RNA markers are used in an algorithm to determine infection type, no more than 25 RNA markers are used in an algorithm to determine infection type, no more than 20 RNA markers are used in an algorithm to determine infection type, no more than 15 RNA markers are used in an algorithm to determine infection type, no more than 10 RNA markers are used in an algorithm to determine infection type, no more than 5 RNA markers are used in an algorithm to determine infection type, no more than 4 RNA markers are used in an algorithm to determine infection type, no more than 3 RNA markers are used in an algorithm to determine infection type or even no more than 2 RNA markers are used in an algorithm to determine infection type.

As well as distinguishing between viral and bacterial infections, the RNA marker combinations described in this application may be used to identify particular types of infection.

Thus, for example the combination of testing CD177, IFI44L and MMP9 is particularly effective at ruling in a virally mediated urinary tract infection or an adenoviral infection.

Thus, for example the combination of testing CD177, IFI44L, MMP9 and PI3 is particularly effective at ruling in a Streptococcus pneumoniae bacterial infection, an RSV infection or a Haemophilus influenza infection.

Table 4 provides exemplary REFSEQ NOs: and exemplary sequences for all the RNA determinants described herein.

TABLE 4

| | | | Exemplary |
|---|---|---|---|
| | | | RNA |
| Gene | | | sequence |
| symbol | REFSEQ NO. | Gene Name | SEQ ID NO: |
| CD177 | NC_000019.10 | CD177 Molecule | 1 |
| | NC_018930.2 | | |
| | NT_011109.17 | | |
| | NM_020406.4 | | |
| IFI44L | NC_000001.11 | interferon | 2 |
| | NT_032977.10 | induced protein | |
| | NC_018912.2 | 44 like | |
| | NM_001375646.1 | | |
| | NM_001375647.1 | | |
| | NM_001375648.1 | | |
| | NM_001375649.1 | | |
| | NM_001375650.1 | | |
| IFIT1 | NC_000010.11 | interferon | 3 |
| | NC_018921.2 | induced protein | |
| | NT_030059.14 | with | |
| | NM_001270927.2 | tetratricopeptide | |
| | NM_001270928.2 | repeats | |
| | NM_001270929.2 | | |
| | NM_001270930.1 | | |
| | NM_001548.5 | | |
| MMP9 | NM_004994.3 | Matrix | 4 |
| PI3 | NC_000020.11 | metallopeptidase | 5 |
| | NC_018931.2 | peptidase inhibitor | |
| | NT_011362.11 | 3 | |
| | NM_002638.4 | | |
| ACSL4 | NM_004458.3 | Acyl-CoA Synthetase | 6 |
| | | Long Chain Family | |
| | | Member 4 | |
| ALPL | NM_000478.6 | Alkaline Phosphatase, | 7 |
| | | Biomineralization | |
| | | Associated | |
| ARG1 | NM_001244438.2 | Arginase 1 | 8 |
| BNIP2 | NM_001320674.2 | BCL2 Interacting | 9 |
| | | Protein 2 | |
| CA4 | NM_000717.5 | Carbonic Anhydrase 4 | 10 |
| EPSTI1 | NM_001002264.4 | Epithelial Stromal | 11 |
| | | Interaction 1 | |
| GYG1 | NM_004130.4 | Glycogenin 1 | 12 |
| HERC5 | NM_016323.4 | HECT And RLD | 13 |
| | | Domain Containing E3 | |
| | | Ubiquitin Protein | |
| | | Ligase 5 | |
| HP | NM_005143.5 | Haptoglobin | 14 |
| HPGD | NM_000860.6 | 15- | 15 |
| | | Hydroxyprostaglandin | |
| | | Dehydrogenase | |
| IFI27 | NM_001130080.3 | Interferon Alpha | 16 |
| | | Inducible Protein 27 | |
| IFI44 | NM_006417.5 | Interferon Induced | 17 |
| | | Protein 44 | |
| IFIT3 | NM_001549.6 | Interferon | 18 |
| | | Induced Protein With | |
| | | Tetratricopeptide | |
| | | Repeats 3 | |
| IFIT5 | NM_012420.3 | Interferon Induced | 19 |
| | | Protein With | |
| | | Tetratricopeptide | |
| | | Repeats 5 | |
| IL18R1 | NM_001282399.2 | Interleukin 18 | 20 |
| | | Receptor 1 | |
| IMPA2 | NM_014214.3 | Inositol | 21 |
| | | Monophosphatase 2 | |
| ISG15 | NM_005101.4 | ISG15 Ubiquitin Like | 22 |
| | | Modifier | |
| LCN2 | NM_005564.5 | Lipocalin 2 | 23 |
| LY6E | NM_001127213.2 | Lymphocyte Antigen 6 | 24 |
| | | Family Member E | |
| MMP8 | NM_002424.3 | Matrix | 25 |
| | | Metallopeptidase 8 | |
| MS4A4A | NM_148975.3 | Membrane Spanning | 26 |
| | | 4-Domains A4A | |
| NLRC4 | NM_021209.4 | NLR Family CARD | 27 |
| | | Domain Containing 4 | |
| ORM1 | NM_000607.4 | Orosomucoid 1 | 28 |
| PGD | NM_002631.4 | Phosphogluconate | 29 |
| | | Dehydrogenase | |
| PGLYRP1 | NM_005091.3 | Peptidoglycan | 30 |
| | | Recognition Protein 1 | |
| RETN | NM_001385726.1 | Resistin | 31 |
| RSAD2 | NM_080657.5 | Radical S-Adenosyl | 32 |
| | | Methionine Domain | |
| | | Containing 2 | |
| S100A12 | NM_005621.2 | S100 Calcium Binding | 33 |
| | | Protein A12 | |
| S100P | NM_005980.3 | S100 Calcium Binding | 34 |
| | | Protein P | |
| SLPI | NM_003064.4 | Secretory Leukocyte | 35 |
| | | Peptidase Inhibitor | |
| SMPDL3A | NM_006714.5 | SMPDL3A | 36 |
| TSPO | NM_000714.6 | Translocator Protein | 37 |
| VNN1 | NM_004666.3 | Vanin 1 | 38 |
| ZDHHC19 | NM_001039617.2 | Zinc Finger DHHC- | 39 |
| | | Type | |
| | | Palmitoyltransferase | |
| | | 19 | |

Additional RNA markers that can be used to assess the severity of viral infection (e.g. coronaviral infection) in patients are disclosed in Tables 5 and 6, herein below.

An upregulation of the amount of RNA set forth in Table 5 above a predetermined amount is indicative of a patient with a severe viral infection. A downregulation of the amount of RNA set forth in Table 6 above a predetermined amount is indicative of a patient with a severe viral infection.

TABLE 5
RNA upregulated in severe patients

CEACAM8
FOLR3
DEFA4
HIST1H4C
IFI27
BPI
GPR84
HP
LTF
MMP8
OLFM4
RETN

TABLE 6
RNA downregulated in severe patients

CPVL
TGFBI
CECR1
IFIT1
IFIT2
ISG15

Additional RNA markers that can be measured are set forth in Table 7 herein below.

TABLE 7

CEACAM1
MTMR11
ZDHHC19
C9orf95
GNA15
BATF
CD5
C3AR1
KIAA1370
TRIB1
MTCH1
CLEC10A
RPGRIP1
HLA-DPB1
HK3
CTSB
GPAA1
TNIP1
PLK1
IFI27
JUP
TST
LAX1
CX3CR1
DEFA4
CD163
CKS2
RGS1
POLD3
PER1
HIF1A
SEPP1
RCBTB2
CBFA2T3
C11orf74
CIT
DHRS7B
LY86
TST
MKI67
KCNJ2
CST3
EMR3

Table 8 summarizes pairs of RNA determinants that can be used to determine the severity of an infectious disease (e.g. a viral disease or a bacterial disease). Typically, in this case, the particular type of infectious disease from which the subject is suffering is unknown.

TABLE 8

| | | | | best | |
|---|---|---|---|---|---|
| Feature | Feature | average | best | component | |
| #1 | #2 | ROC AUC | component | ROC AUC | delta |
| IER3 | SLC7A5 | 0.77 | IER3 | 0.73 | 0.05 |
| RETN | RGS1 | 0.77 | RETN | 0.75 | 0.02 |
| RETN | TGFBI | 0.77 | TGFBI | 0.75 | 0.02 |
| SLC7A5 | UPB1 | 0.76 | UPB1 | 0.71 | 0.05 |
| RETN | SLC7A7 | 0.76 | RETN | 0.75 | 0.02 |
| RGS1 | TGFBI | 0.76 | TGFBI | 0.75 | 0.01 |
| BMX | RETN | 0.76 | RETN | 0.75 | 0.01 |
| RETN | SLC7A5 | 0.76 | RETN | 0.75 | 0.01 |
| RETN | TFRC | 0.76 | RETN | 0.75 | 0.01 |
| NAAA | RETN | 0.76 | RETN | 0.75 | 0.01 |
| IER3 | TFRC | 0.76 | IER3 | 0.73 | 0.03 |
| DEFA3 | TGFBI | 0.76 | TGFBI | 0.75 | 0.01 |
| RETN | SIAH2 | 0.76 | RETN | 0.75 | 0.01 |
| CLEC7A | TGFBI | 0.76 | TGFBI | 0.75 | 0.01 |
| FGFBP2 | TGFBI | 0.76 | TGFBI | 0.75 | 0.01 |
| MMP8 | TGFBI | 0.75 | TGFBI | 0.75 | 0.01 |
| CD4 | RETN | 0.75 | RETN | 0.75 | 0.01 |
| CX3CR1 | RETN | 0.75 | RETN | 0.75 | 0.01 |
| RETN | UPB1 | 0.75 | RETN | 0.75 | 0.01 |
| TGFBI | YOD1 | 0.75 | TGFBI | 0.75 | 0.01 |
| S100P | SLC7A5 | 0.75 | S100P | 0.72 | 0.03 |
| IFI44L | TGFBI | 0.75 | TGFBI | 0.75 | 0.00 |
| CA1 | IER3 | 0.75 | IER3 | 0.73 | 0.03 |
| CX3CR1 | SLC7A5 | 0.75 | CX3CR1 | 0.72 | 0.03 |
| CX3CR1 | YOD1 | 0.75 | CX3CR1 | 0.72 | 0.03 |
| CX3CR1 | TFRC | 0.75 | CX3CR1 | 0.72 | 0.03 |
| CLEC7A | CX3CR1 | 0.75 | CX3CR1 | 0.72 | 0.03 |
| TGFBI | ZBP1 | 0.75 | TGFBI | 0.75 | 0.00 |
| TFRC | UPB1 | 0.75 | UPB1 | 0.71 | 0.04 |
| BMX | TFRC | 0.75 | BMX | 0.69 | 0.06 |
| CX3CR1 | SLC7A7 | 0.75 | CX3CR1 | 0.72 | 0.03 |
| BMX | CX3CR1 | 0.75 | CX3CR1 | 0.72 | 0.02 |
| CX3CR1 | GPR84 | 0.75 | CX3CR1 | 0.72 | 0.02 |
| IER3 | SIAH2 | 0.75 | IER3 | 0.73 | 0.02 |
| CLEC7A | UPB1 | 0.74 | UPB1 | 0.71 | 0.03 |
| CX3CR1 | SIAH2 | 0.74 | CX3CR1 | 0.72 | 0.02 |
| BMX | SLC7A5 | 0.74 | BMX | 0.69 | 0.05 |
| SIAH2 | UPB1 | 0.74 | UPB1 | 0.71 | 0.03 |
| RGS1 | SLC7A7 | 0.74 | SLC7A7 | 0.70 | 0.05 |
| NAAA | UPB1 | 0.74 | NAAA | 0.72 | 0.02 |
| CX3CR1 | NAAA | 0.74 | CX3CR1 | 0.72 | 0.02 |
| CD4 | CX3CR1 | 0.74 | CX3CR1 | 0.72 | 0.02 |
| IER3 | RGS1 | 0.74 | IER3 | 0.73 | 0.01 |
| RGS1 | S100P | 0.74 | S100P | 0.72 | 0.02 |
| NAAA | RGS1 | 0.74 | NAAA | 0.72 | 0.02 |
| BMX | RGS1 | 0.74 | BMX | 0.69 | 0.05 |
| PRTN3 | TFRC | 0.74 | PRTN3 | 0.71 | 0.03 |
| BMX | CLEC7A | 0.74 | BMX | 0.69 | 0.05 |
| CX3CR1 | TYMS | 0.74 | CX3CR1 | 0.72 | 0.02 |
| FGFBP2 | SLC7A7 | 0.74 | SLC7A7 | 0.70 | 0.04 |
| CD4 | SLC7A5 | 0.74 | CD4 | 0.72 | 0.02 |
| CD4 | FGFBP2 | 0.74 | CD4 | 0.72 | 0.02 |

Table 9 summarizes triplets of RNA determinants that can be used to determine the severity of an infectious disease.

TABLE 9

| | | | | | best | |
|---|---|---|---|---|---|---|
| Feature | Feature | Feature | average | | component | |
| #1 | #2 | #3 | ROC AUC | best component | ROC AUC | delta |
| IER3 | RGS1 | SLC7A5 | 0.83 | (‘IER3’, ‘SLC7A5’) | 0.77 | 0.06 |
| RETN | RGS1 | TGFBI | 0.82 | (‘RETN’, ‘RGS1’) | 0.77 | 0.06 |
| RETN | RGS1 | SIAH2 | 0.82 | (‘RETN’, ‘RGS1’) | 0.77 | 0.05 |
| RETN | RGS1 | SLC7A7 | 0.82 | (‘RETN’, ‘RGS1’) | 0.77 | 0.05 |
| RGS1 | S100P | SLC7A5 | 0.82 | (‘S100P’, ‘SLC7A5’) | 0.75 | 0.06 |
| RGS1 | SLC7A5 | UPB1 | 0.82 | (‘SLC7A5’, UPB1’) | 0.76 | 0.05 |
| NAAA | RETN | RGS1 | 0.81 | (‘RETN’, ‘RGS1’) | 0.77 | 0.05 |
| CEP55 | RGS1 | SIAH2 | 0.81 | (‘CEP55’, ‘SIAH2’) | 0.72 | 0.09 |
| IER3 | RGS1 | TFRC | 0.81 | (‘IER3’, ‘TFRC’) | 0.76 | 0.06 |
| CD4 | RETN | RGS1 | 0.81 | (‘RETN’, ‘RGS1’) | 0.77 | 0.05 |
| MMP8 | RGS1 | TGFBI | 0.81 | (‘RGS1’, ‘TGFBI’) | 0.76 | 0.05 |
| RETN | RGS1 | SLC7A5 | 0.81 | (‘RETN’, ‘RGS1’) | 0.77 | 0.05 |
| CD4 | RGS1 | SLC7A5 | 0.81 | (‘CD4’, ‘RGS1’) | 0.74 | 0.07 |
| BMX | RGS1 | TFRC | 0.81 | (‘BMX’, ‘TFRC’) | 0.75 | 0.06 |
| IER3 | RGS1 | SIAH2 | 0.81 | (‘IER3’, ‘SIAH2’) | 0.75 | 0.07 |
| IFI44L | RGS1 | TGFBI | 0.81 | (‘RGS1’, ‘TGFBI’) | 0.76 | 0.05 |
| BMX | RETN | RGS1 | 0.81 | (‘RETN’, ‘RGS1’) | 0.77 | 0.04 |
| BMX | RGS1 | SLC7A5 | 0.81 | (‘BMX’, ‘SLC7A5’) | 0.74 | 0.07 |
| BMX | CIT | SLC7A5 | 0.81 | (‘BMX’, ‘SLC7A5’) | 0.74 | 0.06 |
| RGS1 | SIAH2 | UPB1 | 0.81 | (‘SIAH2’, ‘UPB1’) | 0.74 | 0.06 |
| RGS1 | TGFBI | YOD1 | 0.81 | (‘RGS1’, ‘TGFBI’) | 0.76 | 0.05 |
| IER3 | RGS1 | YOD1 | 0.81 | (‘IER3’, ‘RGS1’) | 0.74 | 0.07 |
| GPR84 | RETN | RGS1 | 0.81 | (‘RETN’, ‘RGS1’) | 0.77 | 0.04 |
| GPR84 | RGS1 | TGFBI | 0.81 | (‘RGS1’, ‘TGFBI’) | 0.76 | 0.05 |
| OLFM4 | RGS1 | TGFBI | 0.81 | (‘RGS1’, ‘TGFBI’) | 0.76 | 0.05 |
| CLEC7A | RGS1 | TGFBI | 0.81 | (‘RGS1’, ‘TGFBI’) | 0.76 | 0.05 |
| BMX | RGS1 | TGFBI | 0.81 | (‘RGS1’, ‘TGFBI’) | 0.76 | 0.04 |
| IFIT1 | RGS1 | TGFBI | 0.81 | (‘RGS1’, ‘TGFBI’) | 0.76 | 0.04 |
| FGFBP2 | RGS1 | TGFBI | 0.81 | (‘RGS1’, ‘TGFBI’) | 0.76 | 0.04 |
| RGS1 | SIAH2 | TGFBI | 0.81 | (‘RGS1’, ‘TGFBI’) | 0.76 | 0.04 |
| DEFA3 | RGS1 | TGFBI | 0.80 | (‘RGS1’, ‘TGFBI’) | 0.76 | 0.04 |
| MMP8 | RGS1 | SLC7A7 | 0.80 | (‘RGS1’, ‘SLC7A7’) | 0.74 | 0.06 |
| GPR84 | RGS1 | SLC7A7 | 0.80 | (‘RGS1’, ‘SLC7A7’) | 0.74 | 0.06 |
| CD4 | MMP8 | RGS1 | 0.80 | (‘CD4’, ‘RGS1’) | 0.74 | 0.06 |
| RGS1 | TGFBI | ZBP1 | 0.80 | (‘RGS1’, ‘TGFBI’) | 0.76 | 0.04 |
| RETN | RGS1 | YOD1 | 0.80 | (‘RETN’, ‘RGS1’) | 0.77 | 0.04 |
| RGS1 | SLC7A5 | TGFBI | 0.80 | (‘RGS1’, ‘TGFBI’) | 0.76 | 0.04 |
| BMX | CIT | TFRC | 0.80 | (‘BMX’, ‘TFRC’) | 0.75 | 0.05 |
| RETN | RGS1 | TFRC | 0.80 | (‘RETN’, ‘RGS1’) | 0.77 | 0.04 |
| IFIT3 | RGS1 | TGFBI | 0.80 | (‘RGS1’, ‘TGFBI’) | 0.76 | 0.04 |
| FGFBP2 | RGS1 | SLC7A7 | 0.80 | (‘RGS1’, ‘SLC7A7’) | 0.74 | 0.06 |
| IFI44L | RETN | RGS1 | 0.80 | (‘RETN’, ‘RGS1’) | 0.77 | 0.03 |
| CD4 | DEFA3 | RGS1 | 0.80 | (‘CD4’, ‘RGS1’) | 0.74 | 0.06 |
| BMX | RGS1 | SLC7A7 | 0.80 | (‘RGS1’, ‘SLC7A7’) | 0.74 | 0.06 |
| BMX | CLEC7A | RGS1 | 0.80 | (‘BMX’, ‘RGS1’) | 0.74 | 0.06 |
| GPR84 | RGS1 | SIAH2 | 0.80 | (‘GPR84’, ‘RGS1’) | 0.74 | 0.06 |
| IFI44L | RGS1 | UPB1 | 0.80 | (‘IFI44L’, ‘UPB1’) | 0.71 | 0.08 |
| LCN2 | RGS1 | TGFBI | 0.80 | (‘RGS1’, ‘TGFBI’) | 0.76 | 0.04 |
| CD4 | OLFM4 | RGS1 | 0.80 | (‘CD4’, ‘RGS1’) | 0.74 | 0.06 |
| IER3 | SLC7A5 | UPB1 | 0.80 | (‘IER3’, ‘SLC7A5’) | 0.77 | 0.02 |
| CA1 | RETN | RGS1 | 0.80 | (‘RETN’, ‘RGS1’) | 0.77 | 0.03 |
| LTF | RGS1 | TGFBI | 0.80 | (‘RGS1’, ‘TGFBI’) | 0.76 | 0.04 |
| CIT | SLC7A5 | UPB1 | 0.80 | (‘SLC7A5’, ‘UPB1’) | 0.76 | 0.03 |
| IFIT2 | RGS1 | TGFBI | 0.80 | (‘RGS1’, ‘TGFBI’) | 0.76 | 0.04 |
| RGS1 | S100P | TGFBI | 0.80 | (‘RGS1’, ‘TGFBI’) | 0.76 | 0.04 |
| BMX | CIT | CLEC7A | 0.80 | (‘BMX’, ‘CLEC7A’) | 0.74 | 0.06 |
| RAP1GAP | RGS1 | TGFBI | 0.80 | (‘RGS1’, ‘TGFBI’) | 0.76 | 0.04 |
| CD4 | GPR84 | RGS1 | 0.80 | (‘CD4’, ‘RGS1’) | 0.74 | 0.05 |
| GPR84 | RGS1 | SLC7A5 | 0.80 | (‘GPR84’, ‘RGS1’) | 0.74 | 0.06 |
| CA1 | IER3 | RGS1 | 0.80 | (‘CA1’, ‘IER3’) | 0.75 | 0.04 |
| IFIT1 | RGS1 | UPB1 | 0.80 | (‘IFIT1’, ‘UPB1’) | 0.72 | 0.07 |
| IFIT1 | RETN | RGS1 | 0.80 | (‘RETN’, ‘RGS1’) | 0.77 | 0.03 |
| CD4 | IFIT3 | RGS1 | 0.80 | (‘CD4’, ‘RGS1’) | 0.74 | 0.05 |
| RETN | RGS1 | S100P | 0.80 | (‘RETN’, ‘RGS1’) | 0.77 | 0.03 |
| RGS1 | S100P | UPB1 | 0.80 | (‘RGS1’, ‘S100P’) | 0.74 | 0.06 |
| CD4 | LTF | RGS1 | 0.80 | (‘CD4’, ‘RGS1’) | 0.74 | 0.05 |
| IFI44 | RGS1 | TGFBI | 0.79 | (‘RGS1’, ‘TGFBI’) | 0.76 | 0.03 |
| CD4 | IFIT2 | RGS1 | 0.79 | (‘CD4’, ‘RGS1’) | 0.74 | 0.05 |
| RGS1 | TFRC | UPB1 | 0.79 | (‘TFRC’, ‘UPB1’) | 0.75 | 0.05 |

For Tables 8 and 9, the direction of the change of the expression level of the RNA determinant is summarized in Table 10. Thus, a “low” expression indicates that the expression level is decreased compared to a control level (e.g. in non-severe subjects); and a “high” expression indicates that the expression level is increased to compared to a control level (e.g. in non-severe subjects).

TABLE 10

| Feature | Direction |
|---|---|
| TGFBI | low |
| RETN | high |
| IER3 | high |
| MMP8 | high |
| CX3CR1 | low |
| CD4 | low |
| S100P | high |
| NAAA | low |
| CEP55 | high |
| UPB1 | high |
| PRTN3 | high |
| ELANE | high |
| GIMAP8 | low |
| TOP2A | high |
| SLC7A7 | low |
| LCN2 | high |
| BMX | high |
| SLC7A5 | high |
| ARG1 | high |
| CEACAM8 | high |
| LY86 | low |
| CEACAM6 | high |
| MPO | high |
| GPR84 | high |
| CD24 | high |
| TFRC | high |
| HLA-DRB1 | low |
| DEFA1B | high |
| OLFM4 | high |
| TCN1 | high |
| LTF | high |
| DEFA3 | high |
| CPVL | low |
| CCR3 | low |
| FGFBP2 | low |
| DEFA4 | high |
| CTSG | high |
| IL7R | low |
| ZDHHC19 | high |
| TYMS | high |
| DEFA1 | high |
| CLEC7A | low |
| SIAH2 | high |
| IFIT2 | low |
| YOD1 | high |
| RAP1GAP | high |
| ZBP1 | low |
| KIAA1324 | low |
| CA1 | high |
| IFIT3 | low |
| CIT | high |
| IFI44L | low |
| C11orf74 | high |
| IFIT1 | low |
| SEPP1 | high |
| IFI44 | low |
| PER1 | high |
| RGS1 | low |
| HIF1A | high |
| TST | high |
| OR52R1 | high |
| CD163 | low |
| KCNJ2 | high |

Table 11 summarizes pairs of RNA determinants that can be used to determine the severity of a purely viral disease.

TABLE 11

| | | | | best | |
|---|---|---|---|---|---|
| Feature | Feature | average | best | component | |
| #1 | #2 | ROC AUC | component | ROC AUC | delta |
| SLC7A5 | TDRD9 | 0.87 | TDRD9 | 0.81 | 0.05 |
| GYG1 | SLC7A5 | 0.86 | GYG1 | 0.82 | 0.05 |
| MMP9 | SLC7A5 | 0.86 | MMP9 | 0.80 | 0.06 |
| HP | SLC7A5 | 0.85 | HP | 0.81 | 0.04 |
| CA4 | SLC7A5 | 0.84 | CA4 | 0.78 | 0.06 |
| CD177 | SLC7A5 | 0.83 | CD177 | 0.81 | 0.02 |
| IL1R2 | TDRD9 | 0.83 | TDRD9 | 0.81 | 0.02 |
| PGLYRP1 | SLC7A5 | 0.83 | PGLYRP1 | 0.76 | 0.07 |
| GYG1 | IL1R2 | 0.83 | GYG1 | 0.82 | 0.01 |
| GYG1 | TPST1 | 0.83 | GYG1 | 0.82 | 0.01 |
| PGLYRP1 | TDRD9 | 0.83 | TDRD9 | 0.81 | 0.01 |
| HP | IL1R2 | 0.83 | HP | 0.81 | 0.02 |
| HP | LGALS2 | 0.83 | HP | 0.81 | 0.02 |
| HP | TPST1 | 0.83 | HP | 0.81 | 0.02 |
| TDRD9 | TPST1 | 0.83 | TDRD9 | 0.81 | 0.01 |
| GPBAR1 | SLC7A5 | 0.83 | GPBAR1 | 0.81 | 0.01 |
| C11orf74 | GYG1 | 0.82 | GYG1 | 0.82 | 0.01 |
| CD177 | TPST1 | 0.82 | CD177 | 0.81 | 0.01 |
| ARG1 | SLC7A5 | 0.82 | ARG1 | 0.77 | 0.05 |
| C11orf74 | TDRD9 | 0.82 | TDRD9 | 0.81 | 0.01 |
| C11orf74 | CD177 | 0.82 | CD177 | 0.81 | 0.01 |
| IL1R2 | MMP9 | 0.82 | MMP9 | 0.80 | 0.02 |
| TDRD9 | TST | 0.82 | TDRD9 | 0.81 | 0.01 |
| S100P | SLC7A5 | 0.82 | S100P | 0.77 | 0.05 |
| RETN | TPST1 | 0.82 | RETN | 0.80 | 0.02 |
| CD177 | IL1R2 | 0.82 | CD177 | 0.81 | 0.01 |
| GPBAR1 | TPST1 | 0.82 | GPBAR1 | 0.81 | 0.01 |
| HP | PGLYRP1 | 0.82 | HP | 0.81 | 0.01 |
| MMP9 | TPST1 | 0.82 | MMP9 | 0.80 | 0.02 |
| LGALS2 | PGLYRP1 | 0.81 | LGALS2 | 0.78 | 0.04 |
| MMP8 | SLC7A5 | 0.81 | MMP8 | 0.80 | 0.01 |
| MMP8 | TPST1 | 0.81 | MMP8 | 0.80 | 0.01 |
| IL1R2 | PGLYRP1 | 0.81 | PGLYRP1 | 0.76 | 0.05 |
| IL1R2 | PER1 | 0.81 | IL1R2 | 0.74 | 0.07 |
| CLIC3 | IL1R2 | 0.81 | CLIC3 | 0.77 | 0.04 |
| ARG1 | CLIC3 | 0.81 | CLIC3 | 0.77 | 0.04 |
| CLIC3 | LGALS2 | 0.81 | LGALS2 | 0.78 | 0.03 |
| LY86 | MMP9 | 0.81 | MMP9 | 0.80 | 0.01 |
| ORM1 | SLC7A5 | 0.81 | ORM1 | 0.76 | 0.05 |
| IL1R2 | LY86 | 0.81 | LY86 | 0.77 | 0.04 |
| LY86 | PGLYRP1 | 0.81 | LY86 | 0.77 | 0.04 |
| DEFA3 | MMP9 | 0.81 | MMP9 | 0.80 | 0.01 |

Table 12 summarizes triplets of RNA determinants that can be used to determine the severity of a purely viral disease.

TABLE 12

| | | | | | best | |
|---|---|---|---|---|---|---|
| Feature | Feature | Feature | average | | component | |
| #1 | #2 | #3 | ROC AUC | best component | ROC AUC | delta |
| IL1R2 | SLC7A5 | TDRD9 | 0.88 | (‘SLC7A5’, ‘TDRD9’) | 0.87 | 0.01 |
| CA4 | GYG1 | SLC7A5 | 0.87 | (‘GYG1’, ‘SLC7A5’) | 0.86 | 0.01 |
| GYG1 | IL1R2 | SLC7A5 | 0.87 | (‘GYG1’, ‘SLC7A5’) | 0.86 | 0.01 |
| ARG1 | GYG1 | SLC7A5 | 0.87 | (‘GYG1’, ‘SLC7A5’) | 0.86 | 0.01 |
| IL1R2 | MMP9 | SLC7A5 | 0.87 | (‘MMP9’, ‘SLC7A5’) | 0.86 | 0.01 |
| GYG1 | PGLYRP1 | SLC7A5 | 0.87 | (‘GYG1’, ‘SLC7A5’) | 0.86 | 0.01 |
| SEPP1 | TDRD9 | TPST1 | 0.86 | (‘TDRD9’, ‘TPST1’) | 0.83 | 0.04 |
| IL1R2 | SEPP1 | TDRD9 | 0.86 | (‘IL1R2’, ‘TDRD9’) | 0.83 | 0.03 |

For Tables 11 and 12, the direction of the change of the expression level of the RNA determinant is summarized in Table 13. A “low” expression indicates that the expression level is decreased compared to a control level (e.g. in non-severe subjects); and a “high” expression indicates that the expression level is increased to compared to a control level (e.g. in non-severe subjects).

TABLE 13

| Feature | Direction |
|---|---|
| GYG1 | high |
| TDRD9 | high |
| CD177 | high |
| GPBAR1 | low |
| HP | high |
| MMP8 | high |
| MMP9 | high |
| RETN | high |
| CA4 | high |
| LGALS2 | low |
| CLIC3 | low |
| LY86 | low |
| ARG1 | high |
| S100P | high |
| PGLYRP1 | high |
| OLFM4 | high |
| ORM1 | high |
| SLPI | high |
| SLC7A5 | high |
| ALPL | high |
| PRF1 | low |
| IL1R2 | high |
| DEFA3 | high |
| FCER1A | low |
| DEFA4 | high |
| HES4 | low |
| CTSG | high |
| LRRN3 | low |
| IFI44L | low |
| ISG15 | low |
| TPST1 | high |
| ZBP1 | low |
| C11orf74 | high |
| CA1 | high |
| TST | high |
| CIT | high |
| HIF1A | high |
| SEPP1 | high |
| KCNJ2 | high |
| RGS1 | low |
| PER1 | low |
| OR52R1 | low |
| CD163 | low |

Table 14 summarizes pairs of RNA determinants that can be used to determine the severity of a bacterial disease (or mixed bacterial/viral disease).

TABLE 14

| | | average | best | best com- | |
|---|---|---|---|---|---|
| Feature | Feature | ROC | com- | ponent | |
| #1 | #2 | AUC | ponent | ROC AUC | delta |
| CIT | SLC7A5 | 0.76 | SLC7A5 | 0.65 | 0.11 |
| RGS1 | TMCC2 | 0.75 | TMCC2 | 0.66 | 0.09 |
| RETN | TMCC2 | 0.72 | RETN | 0.69 | 0.03 |
| RGS1 | SLC7A5 | 0.72 | SLC7A5 | 0.65 | 0.06 |
| RETN | SLC7A5 | 0.71 | RETN | 0.69 | 0.02 |
| LY86 | TGFBI | 0.70 | TGFBI | 0.69 | 0.01 |
| RETN | TGFBI | 0.70 | TGFBI | 0.69 | 0.01 |
| ANKRD22 | TMCC2 | 0.69 | TMCC2 | 0.66 | 0.03 |
| CIT | TMCC2 | 0.68 | TMCC2 | 0.66 | 0.02 |
| MMP8 | TMCC2 | 0.68 | TMCC2 | 0.66 | 0.02 |
| ANKRD22 | SLC7A5 | 0.68 | SLC7A5 | 0.65 | 0.02 |
| DEFA4 | TMCC2 | 0.67 | TMCC2 | 0.66 | 0.01 |
| MMP8 | SLC7A5 | 0.66 | SLC7A5 | 0.65 | 0.01 |
| DEFA4 | SLC7A5 | 0.66 | SLC7A5 | 0.65 | 0.01 |
| ANKRD22 | DEFA1B | 0.65 | DEFA1B | 0.64 | 0.01 |
| DEFA1B | RGS1 | 0.65 | DEFA1B | 0.64 | 0.01 |
| ANKRD22 | KCNJ2 | 0.65 | ANKRD22 | 0.62 | 0.02 |
| ANKRD22 | RAP1GAP | 0.64 | ANKRD22 | 0.62 | 0.02 |

Table 15 summarizes triplets of RNA determinants that can be used to determine the severity of a bacterial disease (or mixed bacterial/viral disease).

TABLE 15

| | | | | | best | |
|---|---|---|---|---|---|---|
| Feature | Feature | Feature | average | | component | |
| #1 | #2 | #3 | ROC AUC | best component | ROC AUC | delta |
| CIT | RETN | SLC7A5 | 0.83 | (‘CIT’, ‘SLC7A5’) | 0.76 | 0.07 |
| RETN | RGS1 | TMCC2 | 0.82 | (‘RGS1’, ‘TMCC2’) | 0.75 | 0.07 |
| RETN | RGS1 | SLC7A5 | 0.80 | (‘RGS1’, ‘SLC7A5’) | 0.72 | 0.08 |
| CIT | MMP8 | SLC7A5 | 0.80 | (‘CIT’, ‘SLC7A5’) | 0.76 | 0.04 |
| ANKRD22 | RGS1 | TMCC2 | 0.79 | (‘RGS1’, ‘TMCC2’) | 0.75 | 0.04 |
| DEFA4 | RGS1 | TMCC2 | 0.78 | (‘RGS1’, ‘TMCC2’) | 0.75 | 0.03 |
| ANKRD22 | RGS1 | SLC7A5 | 0.78 | (‘RGS1’, ‘SLC7A5’) | 0.72 | 0.06 |
| CEACAM8 | RGS1 | TMCC2 | 0.78 | (‘RGS1’, ‘TMCC2’) | 0.75 | 0.03 |
| CIT | HIF1A | SLC7A5 | 0.78 | (‘CIT’, ‘SLC7A5’) | 0.76 | 0.02 |
| RETN | RGS1 | TGFBI | 0.77 | (‘RETN’, ‘TGFBI’) | 0.70 | 0.07 |
| CD163 | CIT | SLC7A5 | 0.77 | (‘CIT’, ‘SLC7A5’) | 0.76 | 0.01 |
| KCNJ2 | RGS1 | SLC7A5 | 0.76 | (‘RGS1’, ‘SLC7A5’) | 0.72 | 0.05 |
| DEFA4 | RGS1 | SLC7A5 | 0.76 | (‘RGS1’, ‘SLC7A5’) | 0.72 | 0.05 |
| MMP8 | RGS1 | TMCC2 | 0.76 | (‘RGS1’, ‘TMCC2’) | 0.75 | 0.01 |
| RGS1 | SLC7A5 | TGFBI | 0.76 | (‘RGS1’, ‘SLC7A5’) | 0.72 | 0.04 |
| RGS1 | TGFBI | TMCC2 | 0.76 | (‘RGS1’, ‘TMCC2’) | 0.75 | 0.01 |
| DEFA1B | RETN | RGS1 | 0.76 | RETN | 0.69 | 0.06 |
| LY86 | RGS1 | TGFBI | 0.75 | (‘LY86’, ‘TGFBI’) | 0.70 | 0.04 |
| ANKRD22 | CIT | TMCC2 | 0.75 | (‘ANKRD22’, ‘TMCC2’) | 0.69 | 0.06 |
| DEFA1B | RGS1 | SLC7A5 | 0.75 | (‘RGS1’, ‘SLC7A5’) | 0.72 | 0.03 |
| RGS1 | SEPP1 | SLC7A5 | 0.74 | (‘RGS1’, ‘SLC7A5’) | 0.72 | 0.03 |
| MMP8 | RGS1 | TGFBI | 0.74 | TGFBI | 0.69 | 0.05 |
| RETN | RGS1 | TST | 0.74 | RETN | 0.69 | 0.05 |
| PER1 | RGS1 | SLC7A5 | 0.74 | (‘RGS1’, ‘SLC7A5’) | 0.72 | 0.02 |
| CPVL | RETN | RGS1 | 0.74 | RETN | 0.69 | 0.05 |
| ANKRD22 | RETN | TMCC2 | 0.74 | (‘RETN’, ‘TMCC2’) | 0.72 | 0.01 |
| RGS1 | SLC7A5 | TST | 0.73 | (‘RGS1’, ‘SLC7A5’) | 0.72 | 0.01 |
| RETN | TGFBI | TMCC2 | 0.72 | (‘RETN’, ‘TMCC2’) | 0.72 | 0.00 |
| DEFA1B | DEFA4 | RGS1 | 0.72 | (‘DEFA1B’, ‘RGS1’) | 0.65 | 0.08 |
| CPVL | MMP8 | RGS1 | 0.72 | MMP8 | 0.64 | 0.08 |
| CD163 | RGS1 | SLC7A5 | 0.72 | (‘RGS1’, ‘SLC7A5’) | 0.72 | 0.01 |
| ANKRD22 | RAP1GAP | RGS1 | 0.72 | (‘ANKRD22’, ‘RAP1GAP’) | 0.64 | 0.08 |
| RETN | SLC7A5 | TGFBI | 0.72 | (‘RETN’, ‘SLC7A5’) | 0.71 | 0.01 |
| ANKRD22 | RETN | SLC7A5 | 0.72 | (‘RETN’, ‘SLC7A5’) | 0.71 | 0.01 |
| MMP8 | RGS1 | SLC7A5 | 0.72 | (‘RGS1’, ‘SLC7A5’) | 0.72 | 0.00 |
| ANKRD22 | KCNJ2 | TMCC2 | 0.71 | (‘ANKRD22’, ‘TMCC2’) | 0.69 | 0.02 |
| CEACAM8 | RGS1 | SLC7A5 | 0.71 | (‘RGS1’, ‘SLC7A5’) | 0.72 | 0.00 |
| ANKRD22 | MMP8 | TMCC2 | 0.71 | (‘ANKRD22’, ‘TMCC2’) | 0.69 | 0.02 |

For Tables 14 and 15, the direction of the change of the expression level of the RNA determinant is summarized in Table 16. A “low” expression indicates that the expression level is decreased compared to a control level (e.g. in non-severe subjects); and a “high” expression indicates that the expression level is increased to compared to a control level (e.g. in non-severe subjects).

TABLE 16

| Feature | Direction |
|---|---|
| TGFBI | low |
| RETN | high |
| TMCC2 | high |
| SLC7A5 | high |
| MMP8 | high |
| DEFA1B | high |
| CEACAM8 | high |
| CPVL | low |
| ANKRD22 | high |
| LY86 | low |
| PER1 | high |
| DEFA4 | high |
| SEPP1 | high |
| RAP1GAP | high |
| OR52R1 | high |
| KCNJ2 | low |
| C11orf74 | high |
| RGS1 | low |
| TST | low |
| CD163 | low |
| CIT | high |
| HIF1A | high |

Specific combinations of RNA markers that can be analysed to diagnose the severity of an infectious disease (in particular viral disease) are listed herein below:
1. CEACAM8, MMP8, SAMSN1 and TGFBI;
2. IL1R2, MMP8, PRC1 and CD74; and
3. DEFA4, IL1R2, MMP8, RETN and LY86.

For the first signature, an increase in CEACAM8, MMP8, SAMSN1 above a predetermined level (e.g. that which is present in the sample of patients with a non-severe infectious disease) and a decrease in the amount of TGFBI below a predetermined level (e.g. that which is present in the sample of patients with a non-severe infectious disease) is indicative of a severe infection.

For the second signature, an increase in IL1R2, MMP8, PRC1 above a predetermined level (e.g. that which is present in the sample of patients with a non-severe infectious disease) and a decrease in the amount of CD74 below a predetermined level (e.g. that which is present in the sample of patients with a non-severe infectious disease) is indicative of a severe infection.

For the third signature, an increase in DEFA4, IL1R2, MMP8, RETN above a predetermined level (e.g. that which is present in the sample of patients with a non-severe infectious disease) and a decrease in the amount of LY86 below a predetermined level (e.g. that which is present in the sample of patients with a non-severe infectious disease) is indicative of a severe infection.

It will be appreciated that as well as determining the level of the RNA markers described herein, the present inventors also contemplate combining these measurements with measurements of protein determinants that are known to be indicative of infection type (e.g. bacterial vs. viral). Examples of proteins that are contemplated by the present invention include those that are described in WO 2013/117746, WO 2011/132086, WO2016/059636 and WO2016/092554, the contents of each are incorporated herein by reference. Other protein determinants contemplated by the present inventors are the protein counterparts of the RNA determinants described herein.

Examples of proteins contemplated by the present inventors include, but are not limited to: TRAIL, CRP, IP-10, MX1, RSAD2 and PCT.

Methods of measuring the levels of proteins are well known in the art and include, e.g., immunoassays based on antibodies to proteins, aptamers or molecular imprints.

The protein determinants can be detected in any suitable manner, but are typically detected by contacting a sample from the subject with an antibody, which binds the protein and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological sample as described above, and may be the same sample of biological sample used to conduct the method described above.

In one embodiment, the antibody which specifically binds the determinant is attached (either directly or indirectly) to a signal producing label, including but not limited to a radioactive label, an enzymatic label, a hapten, a reporter dye or a fluorescent label.

Immunoassays carried out in accordance with some embodiments of the present invention may be homogeneous assays or heterogeneous assays. In a homogeneous assay the immunological reaction usually involves the specific antibody (e.g., anti-determinant antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels, which may be employed, include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al., titled “Methods for Modulating Ligand-Receptor Interactions and their Application,” U.S. Pat. No. 4,659,678 to Forrest et al., titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” The determinant can also be detected with antibodies using flow cytometry. Those skilled in the art will be familiar with flow cytometric techniques which may be useful in carrying out the methods disclosed herein (Shapiro 2005). These include, without limitation, Cytokine Bead Array (Becton Dickinson) and Luminex technology.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I) enzyme labels (e.g., horseradish peroxidase, alkaline phosphatase), and fluorescent labels (e.g., fluorescein, Alexa, green fluorescent protein, rhodamine) in accordance with known techniques.

Antibodies can also be useful for detecting post-translational modifications of determinant proteins, polypeptides, mutations, and polymorphisms, such as tyrosine phosphorylation, threonine phosphorylation, serine phosphorylation, glycosylation (e.g., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF) (Wirth U. and Muller D. 2002).

For determinant-proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant K M using known algorithms, such as the Hill plot, Michaelis-Menten equation, linear regression plots such as Lineweaver-Burk analysis, and Scatchard plot.

Suitable sources for antibodies for the detection of determinants include commercially available sources such as, for example, Abazyme, Abnova, AssayPro, Affinity Biologicals, AntibodyShop, Aviva bioscience, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, against any of the polypeptide determinants described herein.

Kits

Some aspects of the invention also include a determinant-detection reagent such as a combination of oligonucleotides in the form of a kit. The kit may contain in separate containers oligonucleotides directed towards a particular RNA marker, control formulations (positive and/or negative), and/or a detectable label such as fluorescein, green fluorescent protein, rhodamine, cyanine dyes, Alexa dyes, luciferase, radiolabels, among others. The detectable label may be attached to the oligonucleotides. Instructions (e.g., written, tape, VCR, CD-ROM, etc.) for carrying out the assay may be included in the kit.

The kits of this aspect of the present invention may comprise additional components that aid in the detection of the determinants such as enzymes, salts, buffers etc. necessary to carry out the detection reactions.

Thus, according to another aspect of the present invention, there is provided a kit for diagnosing an infection type comprising RNA detection reagents which specifically detect CD177 RNA and IFI44L RNA.

Preferably, the kit contains a number of detection reagents such that no more than 20 RNA markers can be specifically detected.

Preferably, the kit contains a number of detection reagents such that no more than 10 RNA markers can be specifically detected.

Preferably, the kit contains a number of detection reagents such that no more than 5 RNA markers can be specifically detected.

Preferably, the kit contains a number of detection reagents such that no more than 4 RNA markers can be specifically detected.

Preferably, the kit contains a number of detection reagents such that no more than 3 RNA markers can be specifically detected.

Preferably, the kit contains a number of detection reagents such that no more than 2 RNA markers can be specifically detected.

Some aspects of the present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progression to conditions like infection, will be of value in the operations of, for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein.

A machine-readable storage medium can comprise a data storage material encoded with machine readable data or data arrays which, when using a machine programmed with instructions for using the data, is capable of use for a variety of purposes. Measurements of effective amounts of the biomarkers of the invention and/or the resulting evaluation of risk from those biomarkers can be implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system used in some aspects of the invention may also be considered to be implemented as a computer-readable storage medium, configured with a computer program, where the storage medium so configured causes a computer to operate in a specific and predefined manner to perform various functions described herein.

The RNA markers of the present invention, in some embodiments thereof, can be used to generate a “reference RNA marker profile” of those subjects who do not have an infection. The RNA markers disclosed herein can also be used to generate a “subject RNA marker profile” taken from subjects who have an infection. The subject RNA marker profiles can be compared to a reference RNA marker profile to diagnose or identify subjects with an infection. The subject RNA marker profile of different infection types can be compared to diagnose or identify the type of infection. The reference and subject RNA marker profiles of the present invention, in some embodiments thereof, can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

The effectiveness of a treatment regimen can be monitored by detecting RNA marker in an effective amount (which may be one or more) of samples obtained from a subject over time and comparing the amount of RNA marker detected. For example, a first sample can be obtained prior to the subject receiving treatment and one or more subsequent samples are taken after or during treatment of the subject.

For example, the methods of the invention can be used to discriminate between bacterial, viral, healthy, non-infectious and mixed infections (i.e. bacterial and viral co-infections.) This will allow patients to be stratified and treated accordingly.

According to some embodiments of the invention, the method further comprises informing the subject of results of the diagnosis.

As used herein the phrase “informing the subject” refers to advising the subject that based on the diagnosis the subject should seek a suitable treatment regimen.

In a specific embodiment of the invention a treatment recommendation (i.e., selecting a treatment regimen) for a subject is provided by identifying the type of infection (i.e., bacterial, viral, mixed infection or no infection) in the subject according to the method of any of the disclosed methods and recommending that the subject receive an antibiotic treatment if the subject is identified as having bacterial infection or a mixed infection; or an anti-viral treatment is if the subject is identified as having a viral infection.

Antiviral agents which can be used when a subject has been diagnosed as having a viral infection include CRX4 and CCR5 receptor inhibitors such as remdesivir, amantadine and rimantadine and pleconaril. Further antiviral agents that can be used include agents which interfere with viral processes that synthesize virus components after a virus invades a cell. Representative agents include nucleotide and nucleoside analogues that look like the building blocks of RNA or DNA, but deactivate the enzymes that synthesize the RNA or DNA once the analogue is incorporated. Acyclovir is a nucleoside analogue, and is effective against herpes virus infections. Zidovudine (AZT), 3TC, FTC, and other nucleoside reverse transcriptase inhibitors (NRTI), as well as non-nucleoside reverse transcriptase inhibitors (NNRTI), can also be used. Integrase inhibitors can also be used. Other antiviral agents include antisense oligonucleotides and ribozymes (directed against viral RNA or DNA at selected sites).

Some viruses, such as HIV, include protease enzymes, which cleave viral protein chains apart so they can be assembled into their final configuration. Protease inhibitors are another type of antiviral agent that can be used on diagnosis of a viral infection.

The final stage in the life cycle of a virus is the release of completed viruses from the host cell. Some active agents, such as zanamivir (Relenza) and oseltamivir (Tamiflu) treat influenza by preventing the release of viral particles by blocking a molecule named neuraminidase that is found on the surface of flu viruses.

Still other antiviral agents function by stimulating the patient's immune system. Interferons, including pegylated interferons, are representative compounds of this class. Interferon alpha is used, for example, to treat hepatitis B and C. Various antibodies, including monoclonal antibodies, can also be used to target viruses.

When a subject has been diagnosed as having a bacterial infection, anti-bacterial agents may be used to treat the subject.

The antibacterial agent may be bactericidal or bacteriostatic.

In one embodiment, the antibacterial agent is an antibiotic.

As used herein, the term “antibiotic agent” refers to a group of chemical substances, isolated from natural sources or derived from antibiotic agents isolated from natural sources, having a capacity to inhibit growth of, or to destroy bacteria. Examples of antibiotic agents include, but are not limited to; Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, lincosamides, macrolides, monobactams, penicillins, quinolones, sulfonamides, and tetracyclines.

Antibacterial agents also include antibacterial peptides. Examples include but are not limited to abaecin; andropin; apidaecins; bombinin; brevinins; buforin II; CAP18; cecropins; ceratotoxin; defensins; dermas eptin; dermcidin; drosomycin; esculentins; indolicidin; LL37; magainin; maximum H5; melittin; moricin; prophenin; protegrin; and or tachyplesins.

In another embodiment, the methods of the invention can be used to prompt additional targeted diagnosis such as pathogen specific PCRs, chest-X-ray, cultures etc. For example, a diagnosis that indicates a viral infection according to embodiments of this invention, may prompt the usage of additional viral specific multiplex-PCRs, whereas a diagnosis that indicates a bacterial infection according to embodiments of this invention may prompt the usage of a bacterial specific multiplex-PCR. Thus, one can reduce the costs of unwarranted expensive diagnostics.

In a specific embodiment, a diagnostic test recommendation for a subject is provided by identifying the infection type (i.e., bacterial, viral, mixed infection or no infection) in the subject according to any of the disclosed methods and recommending a test to determine the source of the bacterial infection if the subject is identified as having a bacterial infection or a mixed infection; or a test to determine the source of the viral infection if the subject is identified as having a viral infection.

Performance and Accuracy Measures of the Invention.

The performance and thus absolute and relative clinical usefulness of the invention may be assessed in multiple ways as noted above. Amongst the various assessments of performance, some aspects of the invention are intended to provide accuracy in clinical diagnosis and prognosis. The accuracy of a diagnostic or prognostic test, assay, or method concerns the ability of the test, assay, or method to distinguish between subjects having an infection is based on whether the subjects have, a “significant alteration” (e.g., clinically significant and diagnostically significant) in the levels of a determinant. By “effective amount” it is meant that the measurement of an appropriate number of determinants (which may be one or more) to produce a “significant alteration” (e.g. level of expression or activity of a determinant) that is different than the predetermined cut-off point (or threshold value) for that determinant (s) and therefore indicates that the subject has an infection for which the determinant (s) is an indication. The difference in the level of determinant is preferably statistically significant. As noted below, and without any limitation of the invention, achieving statistical significance, and thus the preferred analytical, diagnostic, and clinical accuracy, may require that combinations of several determinants be used together in panels and combined with mathematical algorithms in order to achieve a statistically significant determinant index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. One way to achieve this is by using the Matthews correlation coefficient (MCC) metric, which depends upon both sensitivity and specificity. Use of statistics such as area under the ROC curve (AUC), encompassing all potential cut point values, is preferred for most categorical risk measures when using some aspects of the invention, while for continuous risk measures, statistics of goodness-of-fit and calibration to observed results or other gold standards, are preferred.

By predetermined level of predictability it is meant that the method provides an acceptable level of clinical or diagnostic accuracy. Using such statistics, an “acceptable degree of diagnostic accuracy”, is herein defined as a test or assay (such as the test used in some aspects of the invention for determining the clinically significant presence of determinants, which thereby indicates the presence an infection type) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods predict the presence or absence of an infection or response to therapy with at least 75% total accuracy, more preferably 80%, 85%, 90%, 95%, 97%, 98%, 99% or greater total accuracy.

Alternatively, the methods predict the presence of a bacterial infection or response to therapy with at least 75% sensitivity, more preferably 80%, 85%, 90%, 95%, 97%, 98%, 99% or greater sensitivity.

Alternatively, the methods predict the presence of a viral infection or response to therapy with at least 75% specificity, more preferably 80%, 85%, 90%, 95%, 97%, 98%, 99% or greater specificity. Alternatively, the methods predict the presence or absence of an infection or response to therapy with an MCC larger than 0.2, 0.3, 0.4, 0.5, 0.6, 0.7, 0.8, 0.9 or 1.0.

In general, alternative methods of determining diagnostic accuracy are commonly used for continuous measures, when a disease category has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, California).

In general, by defining the degree of diagnostic accuracy, i.e., cut points on a ROC curve, defining an acceptable AUC value, and determining the acceptable ranges in relative concentration of what constitutes an effective amount of the determinants of the invention allows for one of skill in the art to use the determinants to identify, diagnose, or prognose subjects with a pre-determined level of predictability and performance.

Furthermore, other unlisted biomarkers will be very highly correlated with the determinants (for the purpose of this application, any two variables will be considered to be “very highly correlated” when they have a Coefficient of Determination (R²) of 0.5 or greater). Some aspects of the present invention encompass such functional and statistical equivalents to the aforementioned determinants. Furthermore, the statistical utility of such additional determinants is substantially dependent on the cross-correlation between multiple biomarkers and any new biomarkers will often be required to operate within a panel in order to elaborate the meaning of the underlying biology.

One or more of the listed RNA markers can be detected in the practice of the present invention, in some embodiments thereof. For example, two (2), three (3), four (4), five (5), ten (10), fifteen (15), twenty (20), forty (40), or more RNA markers can be detected.

In some aspects, all RNA markers listed herein can be detected. Preferred ranges from which the number of RNA markers can be detected include ranges bounded by any minimum selected from between one and, particularly two, three, four, five, six, seven, eight, nine ten, twenty, or forty. Particularly preferred ranges include two to five (2-5), two to ten (2-10), two to twenty (2-20), or two to forty (2-40).

Construction of Determinant Panels

Groupings of determinants can be included in “panels”, also called “determinant-signatures”, “determinant signatures”, or “multi-determinant signatures.” A “panel” within the context of the present invention means a group of biomarkers (whether they are determinants, clinical parameters, or traditional laboratory risk factors) that includes one or more determinants. A panel can also comprise additional biomarkers, e.g., clinical parameters, traditional laboratory risk factors, known to be present or associated with infection, in combination with a selected group of the determinants listed herein.

As noted above, many of the individual determinants, clinical parameters, and traditional laboratory risk factors listed, when used alone and not as a member of a multi-biomarker panel of determinants, have little or no clinical use in reliably distinguishing individual normal subjects, subjects at risk for having an infection (e.g., bacterial, viral or co-infection), and thus cannot reliably be used alone in classifying any subject between those three states. Even where there are statistically significant differences in their mean measurements in each of these populations, as commonly occurs in studies which are sufficiently powered, such biomarkers may remain limited in their applicability to an individual subject, and contribute little to diagnostic or prognostic predictions for that subject. A common measure of statistical significance is the p-value, which indicates the probability that an observation has arisen by chance alone; preferably, such p-values are 0.05 or less, representing a 5% or less chance that the observation of interest arose by chance. Such p-values depend significantly on the power of the study performed.

Despite this individual determinant performance, and the general performance of formulas combining only the traditional clinical parameters and few traditional laboratory risk factors, the present inventors have noted that certain specific combinations of two or more determinants can also be used as multi-biomarker panels comprising combinations of determinants that are known to be involved in one or more physiological or biological pathways, and that such information can be combined and made clinically useful through the use of various formulae, including statistical classification algorithms and others, combining and in many cases extending the performance characteristics of the combination beyond that of the individual determinants. These specific combinations show an acceptable level of diagnostic accuracy, and, when sufficient information from multiple determinants is combined in a trained formula, they often reliably achieve a high level of diagnostic accuracy transportable from one population to another.

The general concept of how two less specific or lower performing determinants are combined into novel and more useful combinations for the intended indications, is a key aspect of some embodiments of the invention. Multiple biomarkers can yield significant improvement in performance compared to the individual components when proper mathematical and clinical algorithms are used; this is often evident in both sensitivity and specificity, and results in a greater AUC or MCC. Significant improvement in performance could mean an increase of 1%, 2%, 3%, 4%, 5%, 8%, 10% or higher than 10% in different measures of accuracy such as total accuracy, AUC, MCC, sensitivity, specificity, PPV or NPV. Secondly, there is often novel unperceived information in the existing biomarkers, as such was necessary in order to achieve through the new formula an improved level of sensitivity or specificity. This hidden information may hold true even for biomarkers which are generally regarded to have suboptimal clinical performance on their own. In fact, the suboptimal performance in terms of high false positive rates on a single biomarker measured alone may very well be an indicator that some important additional information is contained within the biomarker results—information which would not be elucidated absent the combination with a second biomarker and a mathematical formula.

On the other hand, it is often useful to restrict the number of measured diagnostic determinants (e.g., RNA markers), as this allows significant cost reduction and reduces required sample volume and assay complexity. Accordingly, even when two signatures have similar diagnostic performance (e.g., similar AUC or sensitivity), one which incorporates less RNAs could have significant utility and ability to reduce to practice. For example, a signature that includes 5 genes compared to 10 genes and performs similarly has many advantages in real world clinical setting and thus is desirable. Therefore, there is value and invention in being able to reduce the number of genes incorporated in a signature while retaining similar levels of accuracy. In this context similar levels of accuracy could mean plus or minus 1%, 2%, 3%, 4%, 5%, 8%, or 10% in different measures of accuracy such as total accuracy, AUC, MCC, sensitivity, specificity, PPV or NPV; a significant reduction in the number of genes of a signature includes reducing the number of genes by 2, 3, 4, 5, 6, 7, 8, 9, 10 or more than genes.

Several statistical and modeling algorithms known in the art can be used to both assist in determinant selection choices and optimize the algorithms combining these choices. Statistical tools such as factor and cross-biomarker correlation/covariance analyses allow more rationale approaches to panel construction. Mathematical clustering and classification tree showing the Euclidean standardized distance between the determinants can be advantageously used. Pathway informed seeding of such statistical classification techniques also may be employed, as may rational approaches based on the selection of individual determinants based on their participation across in particular pathways or physiological functions.

Ultimately, formula such as statistical classification algorithms can be directly used to both select determinants and to generate and train the optimal formula necessary to combine the results from multiple determinants into a single index. Often, techniques such as forward (from zero potential explanatory parameters) and backwards selection (from all available potential explanatory parameters) are used, and information criteria, such as AIC or BIC, are used to quantify the tradeoff between the performance and diagnostic accuracy of the panel and the number of determinants used. The position of the individual determinant on a forward or backwards selected panel can be closely related to its provision of incremental information content for the algorithm, so the order of contribution is highly dependent on the other constituent determinants in the panel.

Construction of Clinical Algorithms

Any formula may be used to combine determinant results into indices useful in the practice of the invention. As indicated above, and without limitation, such indices may indicate, among the various other indications, the probability, likelihood, absolute or relative risk, time to or rate of conversion from one to another disease states, or make predictions of future biomarker measurements of infection. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from determinant results in the relevant training population. Amongst other uses, such formula may be intended to map the feature space derived from one or more determinant inputs to a set of subject classes (e.g. useful in predicting class membership of subjects as normal, having an infection), to derive an estimation of a probability function of risk using a Bayesian approach, or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (RF, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual determinant measurements into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on clinical-determinants such as time from symptoms, gender, race, or sex, where specific formula are used solely on subjects within a class or continuously combining a clinical-determinants as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al., (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al., 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula.

Some determinants may exhibit trends that depends on the patient age (e.g. the population baseline may rise or fall as a function of age). One can use a ‘Age dependent normalization or stratification’ scheme to adjust for age related differences. Performing age dependent normalization, stratification or distinct mathematical formulas can be used to improve the accuracy of determinants for differentiating between different types of infections. For example, one skilled in the art can generate a function that fits the population mean levels of each determinant as function of age and use it to normalize the determinant of individual subjects levels across different ages. Another example is to stratify subjects according to their age and determine age specific thresholds or index values for each age group independently.

In the context of the present invention the following statistical terms may be used:

“TP” is true positive, means positive test result that accurately reflects the tested-for activity. For example in the context of the present invention a TP, is for example but not limited to, truly classifying a bacterial infection as such.

“TN” is true negative, means negative test result that accurately reflects the tested-for activity. For example in the context of the present invention a TN, is for example but not limited to, truly classifying a viral infection as such.

“FN” is false negative, means a result that appears negative but fails to reveal a situation. For example in the context of the present invention a FN, is for example but not limited to, falsely classifying a bacterial infection as a viral infection.

“FP” is false positive, means test result that is erroneously classified in a positive category. For example in the context of the present invention a FP, is for example but not limited to, falsely classifying a viral infection as a bacterial infection.

“Sensitivity” is calculated by TP/(TP+FN) or the true positive fraction of disease subjects.

“Specificity” is calculated by TN/(TN+FP) or the true negative fraction of non-disease or normal subjects.

“Total accuracy” is calculated by (TN+TP)/(TN+FP+TP+FN).

“Positive predictive value” or “PPV” is calculated by TP/(TP+FP) or the true positive fraction of all positive test results. It is inherently impacted by the prevalence of the disease and pre-test probability of the population intended to be tested.

“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test.

“MCC” (Mathews Correlation coefficient) is calculated as follows: MCC=(TP*TN−FP*FN)/{(TP+FN)*(TP+FP)*(TN+FP)*(TN+FN)}\hat{ }0.5 where TP, FP, TN, FN are true-positives, false-positives, true-negatives, and false-negatives, respectively. Note that MCC values range between −1 to +1, indicating completely wrong and perfect classification, respectively. An MCC of 0 indicates random classification. MCC has been shown to be a useful for combining sensitivity and specificity into a single metric (Baldi, Brunak et al. 2000). It is also useful for measuring and optimizing classification accuracy in cases of unbalanced class sizes (Baldi, Brunak et al. 2000).

Often, for binary disease state classification approaches using a continuous diagnostic test measurement, the sensitivity and specificity is summarized by a Receiver Operating Characteristics (ROC) curve according to Pepe et al., “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4 th edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronary Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), Mathews correlation coefficient (MCC), or as a likelihood, odds ratio, Receiver Operating Characteristic (ROC) curve, Area Under the Curve (AUC) among other measures.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value”. Non-limiting examples of “formulas” include sums, ratios, and regression operators, such as coefficients or exponents, biomarker value transformations and normalizations (including, without limitation, those normalization schemes based on clinical-determinants, such as gender, age, or ethnicity), rules and guidelines, statistical classification models, and neural networks trained on historical populations. Of particular use in combining determinants are linear and non-linear equations and statistical classification analyses to determine the relationship between levels of determinants detected in a subject sample and the subject's probability of having an infection or a certain type of infection. In panel and combination construction, of particular interest are structural and syntactic statistical classification algorithms, and methods of index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, and Hidden Markov Models, among others. Other techniques may be used in survival and time to event hazard analysis, including Cox, Weibull, Kaplan-Meier and Greenwood models well known to those of skill in the art. Many of these techniques are useful either combined with a determinant selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Analytical accuracy” refers to the reproducibility and predictability of the measurement process itself, and may be summarized in such measurements as coefficients of variation (CV), Pearson correlation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC and MCC, time to result, shelf life, etc. as relevant.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

In the context of the present invention the following abbreviations may be used: Antibiotics (Abx), Adverse Event (AE), Arbitrary Units (A.U.), Complete Blood Count (CBC), Case Report Form (CRF), Chest X-Ray (CXR), Electronic Case Report Form (eCRF), Food and Drug Administration (FDA), Good Clinical Practice (GCP), Gastrointestinal (GI), Gastroenteritis (GE), International Conference on Harmonization (ICH), Infectious Disease (ID), In vitro diagnostics (IVD), Lower Respiratory Tract Infection (LRTI), Myocardial infarction (MI), Polymerase chain reaction (PCR), Per-oss (P.O), Per-rectum (P.R), Standard of Care (SoC), Standard Operating Procedure (SOP), Urinary Tract Infection (UTI), Upper Respiratory Tract Infection (URTI).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

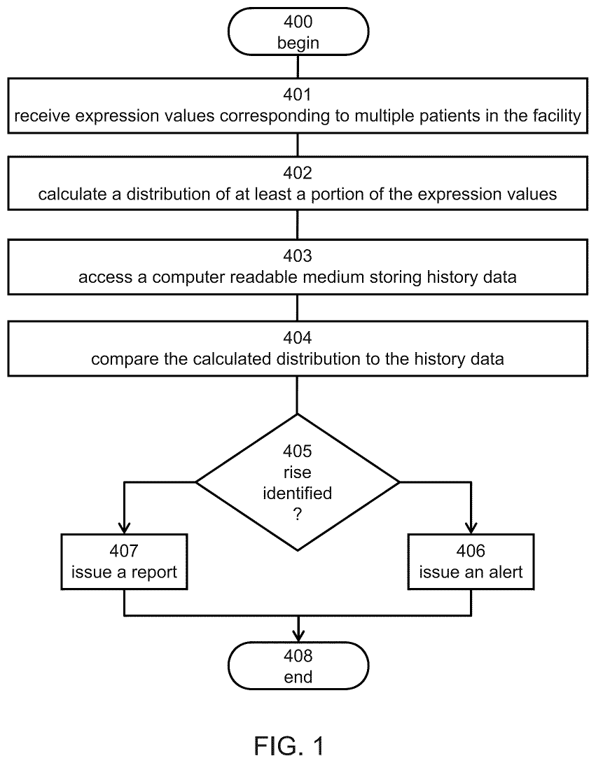

FIG. 1 is a flowchart diagram of a method suitable for identifying an infection outbreak or a change in virulence of an existing pathogen in a medical facility, according to various exemplary embodiments of the present invention. In some embodiments of the present invention the method is also suitable for predicting an infection outbreak or a change in virulence of an existing pathogen in the medical facility. The medical facility can be, for example, a hospital, an urgent care facility, a physician office, a health maintenance organization, or the like.

The method begins at 400 and continues to 401 at which data pertaining to a plurality of determinants derived from multiple patients in the medical facility are received. Preferably, one or more samples (e.g., body liquids, swabs, etc) are/is collected from each of the patients, and the data pertaining to the determinants are obtained separately for each patient. The data can be expression values of the determinants, or some proxies thereof. For example, the method can receive a distribution of classification scores based on the expression values of the determinants.

Typically, the method receives determinant expression values for each of at least 10 or at least 20 or at least 30 or more patients. In some embodiments of the present invention the determinants are proteins, and in some embodiments of the present invention the determinants are RNAs. In some embodiments of the present invention the proteins comprises TRAIL protein, IP10 protein, and CRP protein. Thus, for example, the method can receive, for each patient, an expression value of the TRAIL protein, an expression value of the IP10 protein, and an expression value of the CRP protein, or it can receive the distribution of classification score based on the levels of the TRAIL, IP-10 and CRP.

In some embodiments of the present invention the determinants comprise at least two or at least three or more of the determinants is listed in the Examples section that follows (see, for example, Table 34 of Example 7).

The method proceeds to 402 at which a distribution of at least a portion of the expression values across the medical facility is calculated. Alternatively, the method can receive the distributions as input, without receiving individual protein expression values per patients, in which case 401 can be skipped.

Preferably, the distribution is a statistical distribution. The statistical distribution can be, for example, defined over bins of expression values and indicating the number of patients for which the expression values fall within the respective bin. A representative example of such a distribution is a histogram. Other types of distributions are also contemplated.

In some embodiments of the present invention the distribution is calculated, or received, separately for each protein (e.g., a first distribution describing the expression values of the first protein across the multiple subjects, a second distribution describing the expression values of the second protein across the multiple subjects, etc.).

In some embodiments of the present invention the method calculates a combined distribution for all the proteins. Such a combined distribution can be obtained in more than one way.

When individual expression values per patient are known, a classification score is optionally and preferably calculated per patient, using the expression values obtained for the respective patient, thereby providing multiple scores, and the combined distribution is calculated for the scores, for example, by defining the combined distribution over bins of scores and indicating the number of patients for which the score fall within the respective bin. A representative example of a technique suitable for calculating a classification score using expression values is described in the Examples section that follows (see Example 8).

Measurement of the expression values per patient can be done, at the medical facility using a point of care device configured for receiving a sample from the subject (e.g., a blood sample, a swab or the like) and generating an output pertaining to the expression values. A representative of a point of care device suitable for the present embodiments is marketed by MeMed Diagnostic Ltd., under the tradename MeMed Key™.

The point of care device can communicate with a Laboratory Information System (LIS) associated with the medical facility, for storing the output from the point of care device with the data stored on the computer readable medium of the LIS. The point of care device can alternatively or additionally communicate with a remote server, or a cloud computing facility, or a Hospital Information System (HIS), for storing the output from the point of care device with the data stored on the computer readable medium of the remote server, the cloud computing facility, and/or HIS.

Communication between the point of care device and the above systems, can be wired of wireless according to any communication protocol, such as, but not limited to, Bluetooth, Z-wave, ZigBee, WiFi, Internet, GPRS, GSM, CDMA, 3G, 4G, 5G, and the like.

The point of care device can optionally and preferably also be configured to calculate the classification score per patient based on the measured expression values, and to output the classification score, which can also be stored on the computer readable medium of the LIS.

Optionally, additional medical information per individual patient can also be used for calculating the score. For example, the method can receive data pertaining to at least one of TRAIL, IP-10 or CRP protein levels, calcium levels, chloride levels, cholesterol levels, ferritin levels, lactate levels, hematocrit levels, iron levels, platelet count blood, potassium levels, red blood cell levels, urea levels, and white blood cells levels. The classification score per patient can be calculated from the expression levels of the determinants (e.g., proteins, RNAs). Alternatively, or additionally, a classification score per patient can be a clinical assessment score, for example, SOFA, qSOFA, NEWS, NEWS2, CURB65, APACHE I, APACHE II, APACHE III, SIRS, qCSI, MODS, LODS, 4C deterioration model. The classification score per patient is a optionally and preferably a combined score combining at least two of (i) the score calculated from the expression levels of the determinants, (ii) the clinical assessment score, and (iii) the additional medical information (e.g. age and sex). Such a combined score can be obtained, for example, by a trained machine learning procedure.

When individual information per patient is not available, and distributions are calculated, or received, per protein, providing a protein-specific distribution for each protein. The combined distribution is optionally and preferably cannulated according to a predetermined statistical rule, e.g., a weighted summation of the protein-specific distribution. For example, each protein-specific distribution can be characterized b (y a representative value, such as, but not limited to, a measure of central tendency (e.g., a median, a mean, a mode, etc.), and all the representative values can be summed (e.g., by weighted summation) to provide a combined representative value characterizing the combined distribution. In experiments performed by the present inventors (see Example 6, below), median values of the expression values of each of the proteins were combined, thereby providing a combined median value characterizing the combined distribution of the proteins.

The present embodiments also contemplated calculating the combined distribution by means of a computer-implemented procedure. A representative example of such a procedure is a COVID-19 severity analysis procedure described herein.

At 403, a computer readable medium storing comparative data is accessed, and at 404 the calculated distribution is compared to the comparative data. In some embodiments of the present invention the comparative data comprise history data pertaining to previously received expression values of the proteins, preferably the same proteins, within the medical facility, preferably the same medical facility. The history data is optionally and preferably in the form of one or more protein expression value distributions of the same type or types as calculated at 402.

The comparison is based on the type of distribution that is calculated at 402 and stored in the computer readable medium. For example, when there is a separate distribution for each protein, the comparison is a per-protein comparison, so that a distribution that is calculated, or received, at 402 for a given protein is compared to a sto red history distribution for the same protein. When there is a combined distribution for all proteins, the combined distribution that is calculated or received, at 402 is compared to a combined distribution stored on the medium.

When the computer readable medium contains no history data of the same medical facility, the comparative data can optionally and preferably include history data of one or more other medical facilities. The comparative data can additionally or alternatively include a predefined threshold, or a predefined set of thresholds, that are not history data of the medical facility and are not history data of other medical facilities. While, such threshold or thresholds are not actual history data, they are optionally and preferably derived based on previously performed measurements of the proteins, either at the same medical facility or at other locations.

The comparison can in some embodiments of the present invention be executed between properties characterizing the distributions rather than between the distributions themselves. For example, the comparison can be executed between medians of the distributions, and/or between means of the distributions, and/or between modes of the distributions, and/or between normalized heights of the distributions, and/or between normalized widths (e.g., full-width-at-half-maximum) of the distributions, and/or between normalized areas under the distributions, and the like.

The method proceeds to decision 405 at which the method determines whether there is a significant change (e.g., rise) in the distribution, compared to the comparative data. For example, the method can compare the difference between the distribution (or characterizing property thereof) calculated at 402 and the stored distribution (or characterizing property thereof) to a predetermined threshold, and determine that there is a change when the difference is above the predetermined threshold. The method can also analyze a trend line among time-ordered distributions that are contained in the history data, and determine whether there is a significant change (e.g., rise) by analyzing whether, and to which extent, there is a change in the slope of the trend line once the distribution calculated, or received, at 402 (or characterizing property thereof) is added to the trend line.

When the method determines at 405 that there is a significant change (e.g. rise) in the distribution, the method preferably proceeds to 406 at which an alert that an infection (e.g., a virulent infection) is expected to outbreak across the medical facility, or that a change in virulence of an existing pathogen in the medical facility is expected to occur, is issued. From 406 the method can proceed to 408 at which it ends. When the method determines at 405 that there is no significant rise in the distribution, the method can proceed to end 408, or, alternatively to 407 at which a report pertaining to the comparison is issued.

Method 400 is optionally and preferably, but not necessarily, executed at a central facility that is remote to the medical facility. In these embodiments, the method transmits the alert 406 to a receiving computer at the medical facility. The alert 406 can additionally or alternatively be transmitted to a receiving mobile device (e.g., a smartphone, a smartwatch, a tablet, etc.) of an individual remote from the central facility (e.g., an individual hospitalized or about to be hospitalized at the medical facility, or a family member or physician thereof). The alert can be, for example, displayed on a graphical user interface (GUI) communicating with a cloud computing facility (such as, for example, a web based GUI), on a hospital laboratory information system or mobile device, optionally with an accompanying sound and pop up notification.

The advantage of executing the method at a central facility is that it allows executing the method for a plurality of medical facilities, and transmitting the alert 406 separately to each medical facility, or to one or more individuals. Another advantage is that it allows the method to analyze changes in the distributions across several medical facilities, and to estimate, and optionally and preferably also identify, or predict, a spread of the infection outbreak based on such analysis.

For example, consider a situation in which the method is executed for three medical facilities and determines that an infection is expected to outbreak at a first of the medical facilities but not at the other two facilities. Suppose that in a subsequent execution of the method, the method determines that the infection is also expected to outbreak at a second one of the medical facilities. In this case, the method can determines that the infection outbreak is spreading, and may optionally and preferably transmits an alert pertaining to the spread to the third facility, even though no significant rise in the distribution has been identified in this medical facility.

In some embodiments of the present invention an outbreak score is calculated for the medical facility based on the comparison at 404. In these embodiments, the report 407 or alert 406 can also include the calculated score. The score can indicate the likelihood that for an outbreak of the infection or for a change in the virulence of the existing pathogen across the medical facility. The calculation of the score can be according to a lookup table, prepared in advance, and associating various distributions with outbreak scores. The calculation of the score can alternatively be by thresholding. As a representative example consider an embodiment in which the distribution is characterized by a representative value (e.g., a measure of central tendency). In this case, the outbreak score can be calculated comparing the representative value to a set of thresholds, wherein each threshold range among the set is associated with a specific outbreak score. For example, suppose that there are two thresholds t₁, and t₂, and the representative value for the distribution is d. In this case, when d<t₁ the method can assign an outbreak score of, e.g., 25%, when t₁≤d<t₂ the method can assign an outbreak score of, e.g., 50%, when t₂<d<t₃ the method can assign an outbreak score of, e.g., 75%.

The method of the present embodiments can also be used for allocating treatment resources (e.g., medication, medical devices, vaccines, medical staff, budget) among the medical facilities based of the outbreak score, and optionally and preferably also based on the number of patients in the medical facility. For example, suppose that a first medical facility has a higher outbake score and a more patients than a second facility. In this case, more resources can be allocated to the first facility.

The resource allocation can also be according to the severity of infectious disease calculated for individual patients in the facilities according to the teachings described herein. For example, consider a first medical facility in which there are N₁ patients for which the severity of infectious disease is above some threshold, and a second medical facility in which there are N₂ patients for which the severity of infectious disease is above this threshold. Suppose that N₁>N₂. In this case, more resources are preferably allocated to the first medical facility than to the second medical facility.

The method of the present embodiments can also be used for determining whether to apply public health measures (e.g., quarantine) to the medical facility under analysis. This is typically done based on the likelihood that the infection is expected to outbreak across the medical facility. For example, public health measures can be applied to a medical facility for which the outbreak score is above a predetermined threshold.

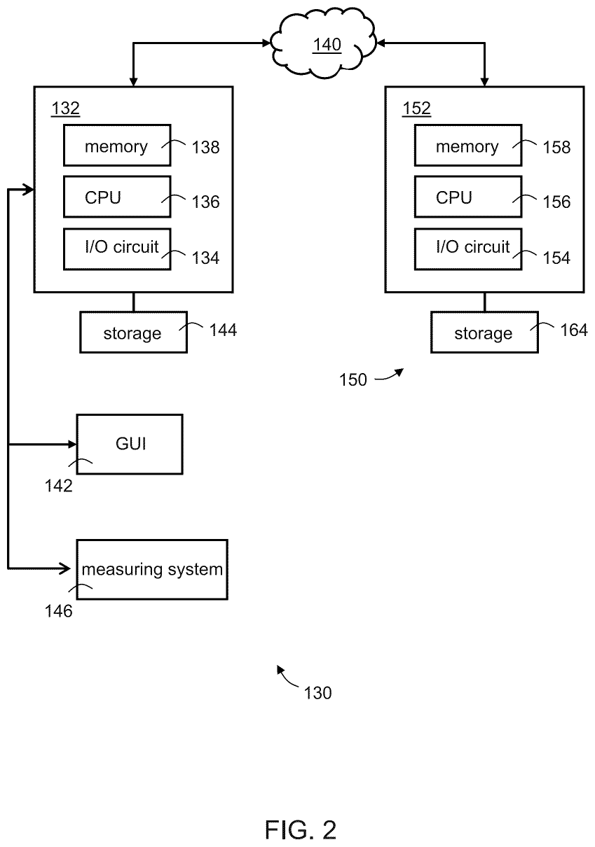

Method 400 can be executed by a client-server computer configuration. A representative example of a client-server computer configuration suitable for the present embodiments is illustrated in FIG. 2. This configuration comprises a client computer 130 having a hardware processor 132, which typically comprises an input/output (I/O) circuit 134, a hardware central processing unit (CPU) 136 (e.g., a hardware microprocessor), and a hardware memory 138 which typically includes both volatile memory and non-volatile memory. CPU 136 is in communication with I/O circuit 134 and memory 138. Client computer 130 preferably comprises a graphical user interface (GUI) 142 in communication with processor 132. I/O circuit 134 preferably communicates information in appropriately structured form to and from GUI 142. This configuration also comprises a server computer 150 which can similarly include a hardware processor 152, an I/O circuit 154, a hardware CPU 156, a hardware memory 158. I/O circuits 134 and 154 of client 130 and server 150 computers can operate as transceivers that communicate information with each other via a wired or wireless communication. For example, client 130 and server 150 computers can communicate via a network 140, such as a local area network (LAN), a wide area network (WAN) or the Internet. Server computer 150 can be in some embodiments be a part of a cloud computing resource of a cloud computing facility in communication with client computer 130 over the network 140. Optionally, a measuring system 146 is associated with client computer 130. Measuring system 146 can measure the amount of the proteins in a body liquid sample of subjects in the medical facility, for example, e.g., using one or more of the techniques described herein.

GUI 142 and processor 132 can be integrated together within the same housing or they can be separate units communicating with each other. Similarly, system 146 and processor 132 can be integrated together within the same housing or they can be separate units communicating with each other.

GUI 142 can optionally and preferably be part of a system including a dedicated CPU and I/O circuits (not shown) to allow GUI 142 to communicate with processor 132. Processor 132 issues to GUI 142 graphical and textual output generated by CPU 136. Processor 132 also receives from GUI 142 signals pertaining to control commands generated by GUI 142 in response to user input. GUI 142 can be of any type known in the art, such as, but not limited to, a keyboard and a display, a touch screen, and the like. In some embodiments, GUI 142 is a GUI of a mobile device such as a smartphone, a tablet, a smartwatch and the like. When GUI 142 is a GUI of a mobile device, the CPU circuit of the mobile device can serve as processor 132 and can execute the code instructions pertaining to the method described herein.

Client 130 and server 150 computers can further comprise one or more computer-readable storage media 144, 164, respectively. Media 144 and 164 are preferably non-transitory storage media storing computer code instructions for executing the method as further detailed herein, and processors 132 and 152 execute these code instructions. The code instructions can be run by loading the respective code instructions into the respective execution memories 138 and 158 of the respective processors 132 and 152.

In embodiments of the present invention, computer 130 and measuring system 146 are local in the medical facility, and computer 150 is at the central facility. The comparative data (e.g., history data pertaining to previously received expression values of the proteins within the medical facility) can be stored, for example, in storage medium 164. The expression values of the proteins can be generated as digital data by local measuring system 146 and can be transmitted to processor 132 by means of I/O circuit 134. Processor 132 can receive the expression values, and transmits the expression values over network 140 to server computer 150. Alternatively, processor 132 can calculate the aforementioned distributions and transmit the distributions themselves over network 140 to server computer 150. In some embodiments of the present invention processor 132 transmits the distributions themselves but does not transmit the expression values.

Computer 150 can calculate, or receive, the distribution as further detailed hereinabove, accesses the comparative data in storage medium 164, and compares the distribution to the comparative data as further detailed hereinabove. When computer 150 identifies that there is a significant change (e.g., rise) in the distribution as further detailed hereinabove, computer 150 can transmit to computer 130, over network 140, an alert that an infection is expected to outbreak across the medical facility, or that a change in virulence of an existing pathogen in a medical facility is expected to occur. Computer 130 receives the alert and displays it on GUI 142.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

An RNA signature of CD177 and IFI44L was tested for its ability to separate patients with bacterial infection from (1) viral infection (B-V), and (2) healthy individuals (B-H). It was also tested to separate patients with bacterial infection from non-infectious illness (B-NI).

Performance was evaluated in 4 datasets (Table 17) that were not used for deriving the signature (“validation datasets”), and the final values represent an average over the datasets, weighted by dataset size.

TABLE 17

| | First | | | | | Patient numbers | | | |
|---|---|---|---|---|---|---|---|---|---|
| Accession | author | Year | Population | Bacteria | Viruses | #B | #V | #NI | #H |
| GSE63990 | Tsalik EL | 2016 | Adults with | Multiple | Multiple | 36 | 58 | 44 | |
| | | | acute | | | | | | |
| | | | respiratory | | | | | | |
| | | | illness at ED | | | | | | |
| GSE73462 | Wright VJ | 2018 | Children at | S. pneumoniae, | Influenza, | 23 | 28 | | 16 |
| | | | pediatric | S. pyogenes, | RSV, | | | | |
| | | | centers with | other | other | | | | |
| | | | febrile | | | | | | |
| | | | illness | | | | | | |
| GSE119217 | Balamuth F | 2020 | Children at ED | | | 28 | 38 | | 12 |
| | | | with suspected | | | | | | |
| | | | sepsis | | | | | | |
| MeMed | | | Children | Gram- | Influenza, | 51 | 79 | | 13 |
| Internal | | | and adults | negative, | other | | | | |
| dataset | | | | Gram- | | | | | |
| | | | | positive, | | | | | |
| | | | | atypical | | | | | |

Performance is expressed as area under the receiver operating characteristic curve (ROC AUC) based on cross-validation of a logistic regression model and is summarized in Table 18. B-NI was tested in 1 data set where it was found that the ROC AUC is 0.79.

TABLE 18

| | B-V | B-H | Average |
|---|---|---|---|
| Number of datasets: | 4 | 3 | |
| ROC AUC: | 0.90 | 0.95 | 0.925 |

The proposed signature is also useful for detecting viral infection. Table 19 below lists performance (in terms of ROC AUC) in separating viral infection from bacterial infection (V-B) and healthy individuals (V-H). V-NI was tested in 1 data set where it was found that the ROC AUC is 0.85.

TABLE 19

| | V-B | V-H | Average |
|---|---|---|---|
| Number of datasets: | 4 | 3 | |
| ROC AUC: | 0.90 | 0.98 | 0.94 |

With the addition of other particular genes, viral infection can be detected earlier. Table 20 summarizes the shortening of the time that passes from infection to detection when IFIT1 is added to the signature. The analysis below is based on data from a viral challenge study, in which subjects were infected (“inoculated”) on purpose.

TABLE 20

| | | CD177, IFI44L, | |
|---|---|---|---|
| | CD177, IFI44L, | IFIT1, MMP9, | |
| Virus | MMP9, PI3 | PI3 | Advantage |
| Influenza A | 36-48 hours | 24-36 hours | 12 hours |
| strain H3N2 | | | |
| Influenza A | 48-60 hours | 36-48 hours | 12 hours |
| strain H1N1 | | | |

Advantage for Diagnostic Test Development:

B-V delta is defined as median expression level of patients with bacterial infection, minus median expression level of patients with viral infection. This measure is calculated for each gene separately. The B-V delta reflects the logarithm of the B-V fold change. For a set of genes, the minimal positive B-V delta (MPD) is the smallest B-V delta over all genes in the set for which the B-V delta is positive. The MPD of the proposed signature (CD177 and IFI44L) is 3.22. The largest MPD among all 146 combinations disclosed in WO2017/149548 was 2.26, as summarized in Table 21. The proposed signature (CD177 and IFI44L) therefore has a 42% advantage over combinations listed in WO2017/149548. This advantage of the proposed signature indicates that it is more amenable to diagnostic test development compared to the combinations listed therein.

TABLE 21

| | Minimal Positive |
|---|---|
| | B-V Delta |
| Signature (CD177 and IFI44L) | 3.22 |
| Pairs and triplets disclosed | 2.26 |
| in WO2017/149548 | |

### Example 2

### 5 Gene Signature—CD177, IFI44L, IFIT1, MMP9 and PI3

Performance of the 5 gene signature was evaluated on the datasets summarized in Table 17. For separating bacterial from viral infection, ROC AUC was in the range 0.86-0.98, with a weighted average of 0.92 (Table 22). Separating bacterial infection from non-infectious illness (based on a single study, GSE63990) reached ROC AUC of 0.77. For separating bacterial infection from healthy individuals, ROC AUC was in the range 0.94-0.99 with a weighted average of 0.98 (Table 23).

TABLE 22

| Study | ROC AUC | Study weight |
|---|---|---|
| GSE73462 | 0.98 | 15% |
| MeMed 2016 | 0.92 | 38% |
| GSE63990 | 0.91 | 28% |
| GSE119217 | 0.86 | 19% |
| Average | 0.92 | |

TABLE 23

| Study | ROC AUC | Study weight |
|---|---|---|
| MeMed 2016 | 0.99 | 45% |
| GSE119217 | 0.99 | 28% |
| GSE73462 | 0.94 | 27% |
| Average | 0.98 | |

To assess signature performance for separating bacterial from viral infection within specific populations, MeMed infectious disease cohort was stratified and performance within the various strata evaluated. Rich phenotyping information was available for the MeMed cohort, enabling stratification by age group, time from symptom onset, physiologic system and clinical syndrome, and detected pathogens. Strata containing five or more patients in both classes (bacterial and viral) were evaluated (Table 24).

TABLE 24

| | | | #B | #V | ROC AUC |
|---|---|---|---|---|---|
| Age group (years) | 0-18 | | 23 | 48 | 0.88 |
| | >18 | | 28 | 31 | 0.93 |
| Time from symptom | 0-2 | days | 30 | 37 | 0.87 |
| ons [symbol] (days) | 3-6 | days | 16 | 32 | 0.87 |
| | 7-14 | days | 5 | 9 | 1.00 |

| | | | | |
|---|---|---|---|---|
| Physiologic System | LRTI | 23 | 17 | 0.91 |
| Clinical Syndrome | URTI | 5 | 25 | 0.99 |
| | GI | 5 | 8 | 0.78 |
| Detected viruses | Influenza virus | 51 | 25 | 0.95 |
| | Respiratory syncytial virus | 51 | 10 | 0.82 |
| | Rhinovirus A/B/C | 51 | 6 | 0.62 |
| | Bocavirus 1/2/3/4 | 51 | 5 | 0.96 |
| | Adenovirus A/B/C/D/E | 51 | 4 | 0.95 |
| Detected bacteria | Streptococcus pneumoniae | 13 | 79 | 0.97 |
| | Escherichia coli | 10 | 79 | 0.95 |
| | Haemophilus influenzae | 5 | 79 | 0.90 |

[symbol]

indicates data missing or illegible when filed

Table 25 summarizes the qualitative pattern of selected genes relative to healthy individuals.

TABLE 25

| | Bacterial | Viral | Non- |
|---|---|---|---|
| | infection | infection | infectious |
| IFI44L | | ↑↑ | |
| IFIT1 | | ↑↑ | |
| PI3 | | ↓ | |
| MMP9 | ↑↑ | ↑ | ↑ |
| CD177 | ↑↑ | ↑ | ↑ |

Arrow pointing up (down) represents high (low) expression level, respectively. Double-arrow represents a stronger effect.

### Example 3

### Example for Using the CD177 and IFI44L Signature for Detecting Bacterial Infection

The proposed gene expression signature can be used to determine whether a patient is infected with bacteria, through the following steps:
1. Measure expression levels of signature genes in the patient's blood
2. Standardize gene expression levels
3. Apply a model to the standardized measurements

### Implementation Examples

Measure Expression Levels of Signature Genes in the Patient's Blood

Extract RNA from a sample of venous blood using an appropriate laboratory protocol. Then use a method for quantitating levels of RNA markers, such as RT-PCR or isothermal amplification. In some methods, the result is a monotonously decreasing function of the RNA level (e.g. RT-PCR)— that is, higher RNA levels result in a lower number; if such a method is used, multiply the result by −1 to obtain a monotonously increasing function.

Standardize Gene Expression Levels

The purpose of this step is to bring the measured gene expression levels to the scale on which the model was trained, before applying the model. An example for a standardization method is a transformation based on the distribution of gene expression levels in the population of healthy individuals. In this example, gene expression levels are measured in a group of healthy individuals using the same method used to measure expression levels in the patient's sample. The healthy group should be large enough to represent the distribution of gene expression levels in the healthy population. For each gene measured, the mean and standard deviation of expression in the healthy group are calculated. Standardization of the patient's gene expression level is performed as follows: first subtracting the mean of the healthy group, and then dividing by its standard deviation. Following standardization, the patient's gene expression level is represented in units of standard deviation of the healthy group, relative to the healthy group mean.

Apply a Model to the Standardized Measurements

A model in this example is an algorithm that classifies a patient as positive or negative for bacterial infection. The model accepts as input standardized expression levels of the signature genes in the patient's blood sample and provides as output a “yes” or “no” answer. There are various types of methods that can be used to learn such a model.

Logistic regression is an example for a method for learning a model. Multinomial logistic regression is a type of logistic regression that can be applied to classify an example into three or more classes. Multinomial logistic regression can be used to learn a model that classifies a patient into one of three classes: bacterial infection, viral infection, or healthy. The learned model constitutes a set of numerical vectors, one for each class; each vector consisting of a coefficient for each gene and a constant term (Table 26 below). Applying the model to a patient's data results in a score ranging from to 1, reflecting the probability that the patient is infected with bacteria. If the score is above a pre-defined threshold, the model output is “yes”; otherwise it is “no”. The threshold is defined based on pre-calculated accuracy measures, such as the required sensitivity for detection of bacterial infection. In the current example, 90% sensitivity is attained by considering patients with a score of 0.15 or higher as positive for bacterial infection.

TABLE 26

| | Bacterial | Viral | Healthy |
|---|---|---|---|
| CD177 | 0.37 | 0.28 | −0.65 |
| IFI44L | −0.27 | 0.72 | −0.45 |
| constant | −0.75 | −0.47 | 1.23 |

Table 26: example for a multinomial logistic regression model based on the signature genes CD177 and IFI44L.

As an example for using the CD177 and IFI44L signature for detecting bacterial infection, consider the following cases presented in Table 27, herein below:

TABLE 27

| Patient | CD177 | IFI44L | Score | Answer |
|---|---|---|---|---|
| 1 | 10.0 | 1.0 | 0.41 | yes |
| 2 | 3.0 | 4.0 | 0.02 | no |
| 3 | 0.0 | 0.0 | 0.10 | no |

Columns “CD177” and “IFI44L” contain the standardized expression levels of these genes in each of the three patients. Column “Score” shows the result of applying the model to the standardized expression levels. For patient 1 the score is 0.41, above the threshold of 0.15, and the answer is therefore positive for bacterial infection. For the other two patients the score is below the threshold, therefore they are found negative for bacterial infection.

### Example 4

### COVID-19 Dataset

Using the dataset from Overmyer K A, Large-scale Multi-omic Analysis of COVID-19 Severity, Cell Systems 2021, genes were identified that showed good performance levels for differentiating patients with severe disease from patients with non-severe disease. Severe disease was defined as requiring mechanical ventilation. The dataset is summarized in Table 28.

TABLE 28

| | | | Sample | | # | # Non- |
|---|---|---|---|---|---|---|
| Accession | Pathogen | Population | type | Outcome | Severe | Severe |
| GSE157103 | SARS- | Adults | Blood | Mechanical | 42 | 58 |
| | Cov-2 | admitted to | | ventilation | | |
| | | hospital | | | | |

For each gene in the dataset, ROC area under curve (ROC AUC) and fold change between the median of the severe group and the median of the non-severe group were calculated. Genes elevated in the severe group (ROC AUC>0.8, fold change>2) ordered by fold change in decreasing order are listed in Table 29, herein below.

TABLE 29
Gene

OLAH
IL1R2
CD177
PFKFB2
ARG1
GRB10
ASPH
LRRC70
VNN1
IL1R1
IRAK3
S100A12
NSUN7
UGCG
GADD45A
CA4
ANXA3
GYG1
ST6GALNAC3
BMX
SRPK1
PYGL
PCOLCE2
MGAM
CYSTM1

Genes decreased in the severe group (ROC AUC>0.8, fold change<0.5) ordered by fold change in increasing order are listed in Table 30, herein below.

TABLE 30
Gene

GNLY
CD4
HLA-DPB1
HLA-DPA1
ZAP70
CLEC10A
PTPRCAP
CD6
XCL2
GIMAP5
HLA-DQB1
CD3D
IL32
HLA-DRB1
GZMM
CD2
PRF1
GZMB
MAL
LCK
PID1
CD160
SIGIRR
CD52
GZMH
TNFRSF25
CCR5
C12orf57
CD247
CCR7
CD7
MATK
RPL36
FLT3LG
SIRPG
PLAAT4
RPS28
TBX21
TRABD2A
LFNG
SPOCK2
NCR3
CD3E
CYB561
RPS19
CD5
HLA-DRA
HLA-DMB
TLE5
MAD1L1

Performance of pairs of RNA determinants on the COVID-19 dataset are summarized in Tables 31.

TABLE 31

| | GSE157103 | |
|---|---|---|
| Gene pair | ROC AUC | Table No. |
| GYG1, SLC7A5 | 0.86 | Viral (Table 11) |
| GYG1, IL1R2 | 0.86 | Viral (Table 11) |
| BMX, CX3CR1 | 0.85 | All (Table 8) |
| GYG1, TPST1 | 0.84 | Viral (Table 11) |
| CD177, IL1R2 | 0.84 | Viral (Table 11) |
| HP, IL1R2 | 0.84 | Viral (Table 11) |
| GPBAR1, TPST1 | 0.84 | Viral (Table 11) |
| BMX, RETN | 0.84 | All (Table 8) |
| TGFBI, YOD1 | 0.84 | Viral (Table 11) |
| CLIC3, IL1R2 | 0.84 | All (Table 8) |
| BMX, CLEC7A | 0.83 | All (Table 8) |
| CX3CR1, SIAH2 | 0.83 | All (Table 8) |
| BMX, RGS1 | 0.83 | All (Table 8) |
| GPBAR1, SLC7A5 | 0.83 | Viral (Table 11) |

### Example 5

Analysis of various datasets enabled the present inventors to uncover additional signatures capable of quantitating the severity of infectious diseases.

These signatures are summarized in Table 32, herein below.

TABLE 32

| | Elevated genes | Decreased genes |
|---|---|---|
| Signature 1 | CEACAM8, MMP8, SAMSN1 | TGFBI |
| Signature 2 | IL1R2, MMP8, PRC1 | CD74 |
| Signature 3 | DEFA4, IL1R2, MMP8, RETN | LY86 |

The signatures were tested on datasets summarized in Table 33.

TABLE 33

| | Viral/ | Signature 1 | Signature 2 | Signature 3 |
|---|---|---|---|---|
| Dataset | Non-viral | ROC AUC | ROC AUC | ROC AUC |
| GSE61821 | Viral | 0.81 | 0.93 | 0.92 |
| GSE54514 | Non-viral | 0.96 | 0.90 | 0.77 |
| GSE101702 | Viral | 0.90 | 0.85 | 0.84 |

### Example 6

### Identification of Outbreak

Measurements of TRAIL, CRP and IP-10 were performed on an infectious population across 6 different hospitals encompassing patients with diverse infectious etiology, sex and age. Median values of the expression levels each of the proteins TRAIL, IP-10 and CRP were collected from each hospital, and were combined to provide a combined median value per hospital. The combined median values were analyzed to identify clusters across hospitals.

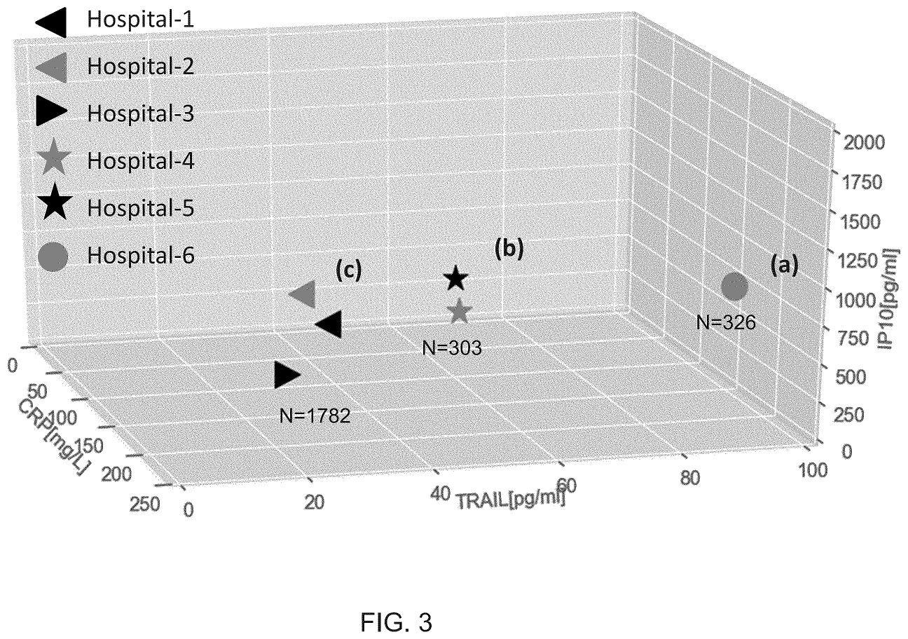

The results are shown in FIG. 3. Three clusters were identified. Cluster (a) included a population of patients found to be negative to SARS-CoV-2 pathogen. This cluster includes a single hospital, referred to as hospital-6. Patients in this cluster were considered as controls exhibiting a normal protein distribution. Cluster (b) included a mixed population of patients found to be either negative or mild positive to SARS-CoV-2. Patients in this cluster exhibited a change in the TRAIL, IP-10 and CRP median distributions, compared to the control group (hospital-6) distributions. Cluster (c) Cluster (b) included a population of patients found to be positive to SARS-CoV-2, with severe illness. Patients in this cluster exhibited a significant change in the TRAIL, IP-10, CRP median distribution.

This Example demonstrates that by analyzing the distributions of the expression values (combined medians values, in this Example), the present embodiments can identify infection outbreak, or a change in virulence of an existing pathogen.

### Example 7

### List of Determinants Suitable for Identifying an Outbreak

A list of determinants suitable for identifying an outbreak, according to some embodiments of the present invention is provided in Table 34, below.

TABLE 34
TRAIL

IP-10
CRP
IL-6
PCT
NGAL
Pro-adrenomedullin
IL1R/IL1R1/IL1RA
SAA/SAA1
sTREM1/TREM1
sTREM2/TREM2
RSAD2
NGAL
IL1R/IL1R1/IL1RA
IL-1-beta
SAA/SAA1
SAA/L
MMP8
MX1
Neopterin
Presepsin
TNF-alpha
IL-10
IL-8
IL-7
MCP-1
MCP-3
MIP-1-alpha
G-CSF

### Example 8

### Exemplified Technique for Calculating a Classification Score

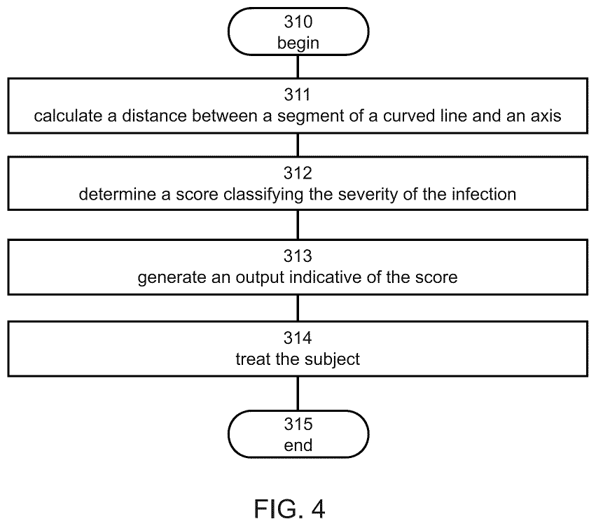

FIG. 4 is a flowchart diagram of a method suitable for calculating a classification score based on biological data obtained from a subject, according to various exemplary embodiments of the present invention. The method described in this Example is particularly useful to calculate a score pertaining to the severity of a coronaviral (e.g., COVID19) infection, but similar techniques can be applied for calculating score pertaining to the severity of other diseases, or other classification scores.

The biological data analyzed by the method contain expression values of a one or more determinants, such as the determinants described herein. In some embodiments the biological data comprises expression values of three or more proteins or RNAs. In some embodiments the biological data comprises expression values of at least CRP, TRAIL and IP-10. According to a particular embodiment, the levels of secreted (i.e. soluble) proteins (e.g., TRAIL, CRP and IP-10) are analyzed by the method.

Referring to FIG. 4, the method begins at 310 and continues to 311 at which a distance d between a segment S_{ROI} of a curved line S and a non-curved axis π is calculated.

The axis π is defined by a direction. The distance between the segment of line S and axis π is calculated at a point P over the axis π. P is defined by a coordinate denoted δ.

It is recognized that in the context of lines, coordinates are numbers that determine positions on axes that are defined by directions. Usually, the terms “coordinate”, “axis”, and “direction” are interchanged in the literature, but they actually represent different (but related) mathematical objects. For example, in a Cartesian coordinate system, the letter x, is used to denote (i) the horizontal axis, (ii) the rightward direction on this axis, and (iii) a point on this axis.

A formulation for obtaining the value of the coordinate δ is provided below. The formulation when considered generally (namely as a function and not as a value returned by the function) defines the direction along which the axis π extends. Below, the direction along which π extends is are denoted using the same Greek letters as the coordinate δ, except that the direction is denoted by an underlined Greek letter to indicate that it is a vector quantity. In this notation, the axis π extends along direction δ.

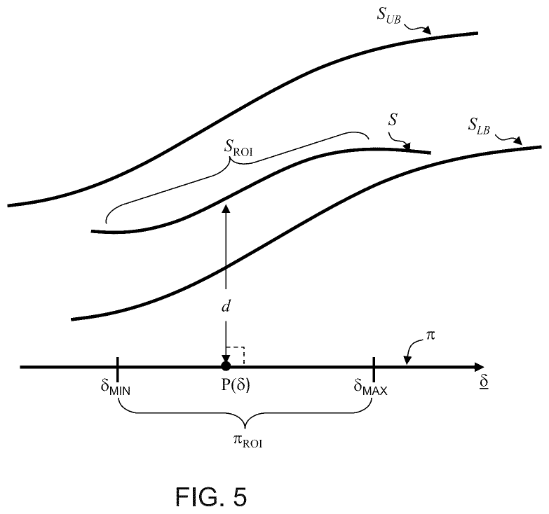

FIG. 5 illustrates the axis π along direction δ. Also shown is a point P at coordinate δ. On axis π, along direction δ, there is a region-of-interest π_{ROI} which is a linear segment of axis π spanning from a minimal coordinate δ_{MIN} to a maximal coordinate δ_{MAX} along direction δ. The point P is within the region-of-interest π_{ROI}.

The distance d, calculated at 311, is measured from S to the point P, perpendicularly to π. The segment S_{ROI} of S is above the region-of-interest π_{ROI}. In other words, π_{ROI} is the projection of S_{ROI} on π. S_{ROI} is preferably a curved segment of the curve S.

The coordinate is defined by a combination of expression values of the determinants (e.g., proteins, RNAs). For example, δ can be a combinations of the expression values, according to the following equation:

δ=C₀+C₁D₁+C₂D₂+ . . . +ϕ

where C₀, C₁, C₂, . . . are constant and predetermined coefficients, each of the variables D₁, D₂, . . . is an expression value of one of the determinants (e.g., D₁ can be the expression value of the TRAIL protein, D₂ can be the expression value of the IP-10 protein, and D₃ can be the expression value of the CRP protein), and ϕ is a function that is nonlinear with respect to at least one of the expression values. C₀ is referred to as an offset coefficient.

In some embodiments of the present invention, prior to the calculation of the coordinate δ, each of the expression values is compared to one or more respective thresholds. In these embodiments, the coordinate δ is defined at least based on the result of this comparison. For example, each of the expression values can be compared to an upper threshold and a lower threshold, wherein when the respective expression value is within a range defined by the lower and upper thresholds, the respective expression value is used for calculating the coordinate δ, when the respective expression value is outside this range but less than the lower threshold, the lower threshold is used for calculating the coordinate δ instead of the respective expression value, and when the respective expression value is outside this range but more than the upper threshold, the upper threshold is used for calculating the coordinate δ instead of the respective expression value.

The lower threshold for the TRAIL protein is denoted TRAILₘᵢₙ, and the upper threshold for the TRAIL protein is denoted TRAILₘₐₓ. Typical values for TRAILₘᵢₙ are from about 0 to about 20 pg/ml, e.g., about 15 pg/ml, and typical values for TRAIL max are from about 250 to about 350 pg/ml, e.g., about 300 pg/ml.

The lower threshold for the IP-10 protein is denoted IP10ₘᵢₙ, and the upper threshold for the IP-10 protein is denoted IP10ₘₐₓ. Typical values for IP10ₘᵢₙ are from about 0 to about 150 pg/ml, e.g., about 100 pg/ml, and typical values for IP10ₘₐₓ are from about 5000 to about 7000 pg/ml, e.g., about 6000 pg/ml.

The lower threshold for the CRP protein is denoted CRPₘᵢₙ, and the upper threshold for the CRP protein is denoted CRPₘₐₓ. Typical values for CRPₘᵢₙ are from about 0 to about 1.5 mg/L, e.g., about 1 mg/L, and typical values for CRP max are from about 200 to about 300 mg/L, e.g., about 250 mg/L.

The present embodiments also contemplate an operation in which each of the expression values is compared a confidence range of values, wherein when the respective expression value is outside the confidence range, the method issues an error or warning message that the respective expression value is outside the confidence range. A typical confidence range for the TRAIL protein is from about −5 to about 1,000 pg/ml, a typical confidence range for the IP-10 protein is from about −50 to about 30,000 pg/ml, and a typical confidence range for the CRP protein is from about −10 to about 1,000 mg/L. Other confidence ranges are also contemplated.

The function ϕ is optional and may be set to zero (or, equivalently, not be used in the calculation of the coordinate δ). When ϕ=0 the coordinate δ is a linear combination of the determinants (e.g., proteins, RNAs). The nonlinear function ϕ can optionally and preferably be expressed as a sum of powers of the expression values, for example, according to the following equation:

ϕ=ΣᵢqᵢXᵢ^{γi}

where i is a summation index, qᵢ is a set of coefficients, Xᵢ∈{D₁, D₂, . . . }, and γi are numerical exponents. Note that the number of terms in ϕ does not necessarily equals the number of the determinant (e.g., proteins, RNAs), and that two or more terms in the sum may correspond to the same determinant, albeit with a different numerical exponent.

A representative example of the offset coefficient C₀, suitable for the present embodiments, is from about 0.9−κ to about 0.9+κ, where κ is defined as (log(pₘₐₓ/(1−pₘₐₓ))−log(pₘᵢₙ/(1−pₘᵢₙ))), where pₘₐₓ is a predetermined parameter which is more than 0.9 and less than 1 (e.g., 0.99), and pₘᵢₙ is a predetermined parameter from about 0.01 to about 0.5. For example, when pₘₐₓ is about and pₘᵢₙ is about 0.01, C₀ is from about −8 to about 10, when pₘₐₓ is about 0.99 and pₘᵢₙ is about 0.2, C₀ is from about −5 to about 6.9, and when pₘₐₓ is about 0.99 and N In is about 0.5, C₀ is from about −3.67 to about 5.5. In some embodiments of the present invention C₀ is about 0.93.

Herein, “log” means a natural logarithm.

A representative example of the coefficient C₁, suitable in embodiments in which C₁ is a coefficient of the expression value of the TRAIL protein, is from about −0.04-κ/TRAILₘᵢₙ ml/pg to about −0.04+κ/TRAILₘᵢₙ ml/pg, where κ and TRAILₘᵢₙ are as indicated above. For example, when pₘₐₓ is about 0.99, pₘᵢₙ is about 0.01 and TRAILₘᵢₙ is about 15 pg/ml, C₁ is from about −0.65 ml/pg to about 0.57 ml/pg, when pₘᵢₙ is about 0.99, pₘᵢₙ is about 0.2 and TRAILₘᵢₙ is about 15 pg/ml, C₁ is from about −0.44 ml/pg to about 0.36 ml/pg, and when pₘᵢₙ is about 0.99, pₘᵢₙ is about and TRAILₘᵢₙ is about 15 pg/ml, C₁ is from about −0.35 ml/pg to about 0.27 ml/pg. In some embodiments of the present invention C₁ is about −0.041 ml/pg.

A representative example of the coefficient C₂, suitable in embodiments in which C₂ is a coefficient of the expression value of the IP-10 protein, is from about 0.0006-κ/IP10ₘᵢₙ ml/pg to about 0.0006+κ/IP10ₘᵢₙ ml/pg, where κ and IP10ₘᵢₙ are as indicated above. For example, when Amax is about 0.99, pₘᵢₙ is about 0.01 and IP10ₘᵢₙ is about 100 pg/ml, C₂ is from about −0.1 ml/pg to about 0.1 ml/pg, when pₘᵢₙ is about 0.99, pₘᵢₙ is about 0.2 and IP10ₘᵢₙ is about 100 pg/ml, C₂ is from about −0.06 ml/pg to about 0.06 ml/pg, and when pₘᵢₙ is about 0.99, pₘᵢₙ is about 0.5 and IP10ₘᵢₙ is about 100 pg/ml, C₂ is from about −0.04 ml/pg to about 0.05 ml/pg. In some embodiments of the present invention C₂ is about 0.0006 ml/pg.

A representative example of the coefficient C₃, suitable in embodiments in which C₃ is a coefficient of the expression value of the CRP protein, is from about 0.003-κ/CRPₘᵢₙ L/mg to about 0.003+κ/CRPₘᵢₙ L/mg, where pₘₐₓ, pₘᵢₙ and CRPₘᵢₙ are as indicated above. For example, when pₘₐₓ is about 0.99, pₘᵢₙ is about 0.01 and CRPₘᵢₙ is about 1 mg/L, C₃ is from about −9.19 L/mg to about 9.19 L/mg, when pₘₐₓ is about 0.99, pₘᵢₙ is about 0.2 and CRPₘᵢₙ is about 1 mg/L, C₃ is from about −5.98 L/mg to about 5.98 L/mg, and when pₘₐₓ is about 0.99, pₘᵢₙ is about 0.5 and CRPₘᵢₙ is about 1 mg/L, C₃ is from about −4.6 L/mg to about 4.6 L/mg. In some embodiments of the present invention C₃ is about 0.0025 L/mg.

The boundaries δ_{MIN}, and δ_{MAX} of π_{ROI} preferably correspond to the physiologically possible ranges of the expression values of the determinant (e.g., proteins, RNAs) according to a protocol used for measuring those expression values. For example, in measurements performed by the Inventors using an ELISA protocol, typical physiologically possible ranges are from 0 to about 400 ug/ml for CRP, from 0 to about 3000 pg/ml for IP-10, and from 0 to about 700 pg/ml for TRAIL. Some subjects may exhibit concentrations that lie outside these ranges. It is appreciated that when the expression values of TRAIL, CRP and IP-10 are measured by other techniques or protocols the values of the boundaries δ_{MIN}, and δ_{MAX} of π_{ROI}, and optionally also the coefficients C₀, C₁, C₂, C₃, may be different that the above exemplified values.

At least a major part of the segment S_{ROI} of curved line S is between two curved lines referred to below as a lower bound curved line S_{LB} and an upper bound curved line S_{UB}.

As used herein “major part of the segment S_{ROI}” refers to a part of a smoothed version S_{ROI} whose length is 60% or 70% or 80% or 90% or 95% or 99% of a smoothed version of the length of S_{ROI}.

As used herein, “a smooth version of the segment S_{ROI}” refers to the segment S_{ROI}, excluding regions of S_{ROI} at the vicinity of points at which the Gaussian curvature is above a curvature threshold, which is X times the median curvature of S_{ROI}, where X is 1.5 or 2 or 4 or 8.

The following procedure can be employed for the purpose of determining whether the major part of the segment S_{ROI} is between S_{LB} and S_{UB}. Firstly, a smoothed version of the segment S_{ROI} is obtained. Secondly, the length A₁ of the smoothed version of the segment S_{ROI} is calculated. Thirdly, the length A₂ of the part of the smoothed version of the segment S_{ROI} that is between S_{LB} and S_{UB} is calculated. Fourthly, the percentage of A₂ relative to A₁ is calculated.

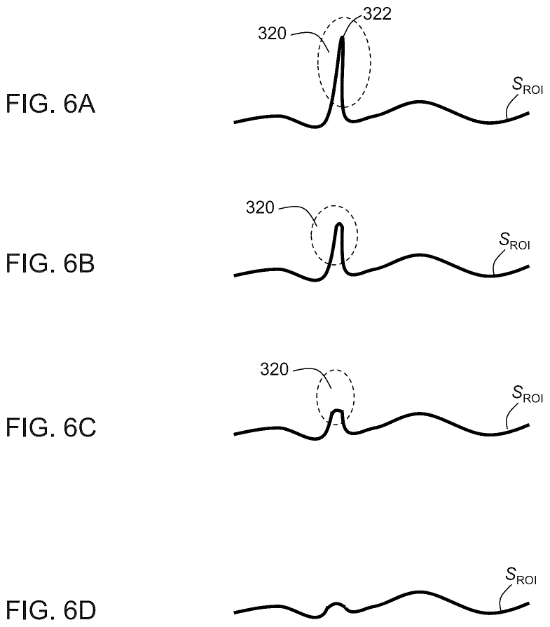

FIGS. 6A-D illustrates a procedure for obtaining the smooth version of S_{ROI}. The Gaussian curvature is calculated for a sufficient number of sampled points on S_{ROI}. For example, when the line is represented as a set of points, the Gaussian curvature can be calculated for the points in the set. The median of the Gaussian curvature is then obtained, and the curvature threshold is calculated by multiplying the obtained median by the factor X. FIG. 6A illustrates S_{ROI} before the smoothing operation. Marked is a region 320 having one or more points 322 at which the Gaussian curvature is above the curvature threshold. The point or points at which the Gaussian curvature is maximal within region 320 is removed and region 320 is smoothly interpolated, e.g., via polynomial interpolation (FIG. 6B). The removal and interpolation is repeated iteratively (FIG. 6C) until the segment S_{ROI} does not contain regions at which the Gaussian curvature is above the curvature threshold (FIG. 6D).

The lower and upper bound curved lines S_{LB} and S_{UB} can be written in the form:

S_{LB}=f(δ)-ε₀,

S_{UB}=f(δ)+ε₁

where f(δ) is a classification function of the coordinate δ (along the direction δ) which represents the severity of a coronaviral infection of the subject. In some embodiments of the invention f(δ) comprises a multiplication between a saturation function p of the coordinate δ, and a saturation function w of the expression value of the CRP protein.

A saturation function, as used herein, is a monotonically increasing function which exhibits a plateau for sufficiently large value of its argument. The plateau is preceded by a segment at which the second derivative of the saturation function changes its sign. At the plateau, the first derivative of the saturation function monotonically decreases for any value of the argument above a predetermined value.

In some embodiments of the present invention the saturation function p of the δ coordinate is linearly proportional to 1/(1+Exp(−δ)). In some embodiments of the present invention p=1/(1+Exp(−δ)). In these embodiments, setting the coefficients C₀, C₁, etc., based on the predetermined parameters pₘᵢₙ and pₘₐₓ as further detailed hereinabove, ensures that that a change of the coefficient value to the upper limit of the respective range or to the lower limit of the respective range translates to a change in p from pₘᵢₙ to pₘₐₓ.

In some embodiments of the present invention the saturation function w of the expression value of the CRP protein is linearly proportional to 1/(1+(CRP/CRP₀)^{-h}), where CRP₀ and h are predetermined shift and width parameters. Representative examples of a value for the shift parameter CRP₀ is from about 1 mg/L to about 1000 mg/L, or from about 100 mg/L to about 500 mg/L, or from about 200 mg/L to about 300 mg/L, e.g., about 260 mg/L. Representative examples of a value for the width parameter h is from about 2 to about 100, or from about 2 to about 50, or from about 2 to about 10, e.g., about 6. In some embodiments of the present invention w=1/(1+(CRP/CRP₀)^{-h}).

The classification function optionally and preferably also includes at least one term that does not depend on w and/or at least one term that does not depend on p. In experiments performed by the Inventors it was found that when the classification function is in the form f(δ), p(1−w)+w, a more accurate classification of the severity of the coronaviral infection is obtained.

In any of the above embodiments each of the parameters C₀ and C₁ is less than 0.5 or less than 0.4 or less than 0.3 or less than 0.2 or less than 0.1 or less than 0.05.

Referring again to FIG. 4, the method proceeds to 312 at which a score classifying the severity of a coronaviral infection of the subject is determined based on the distance d. For example, the score can be the calculated distance or some proxy thereof, e.g., a value that is proportional to d. In some embodiments, the score is defined as Round(100·d), where Round is a function that returns the nearest integer of its argument (e.g., Round(40.1)=40, and Round(40.51)=41).

The method optionally and preferably continues to 313 at which an output indicative of the score is generated. The output can be presented as text, and/or graphically and/or using a color index.

The output can in some embodiments of the present invention be a description of the score, rather than the score itself. As a representative example, which is not to be considered as limiting, when the score ranges from 0 to 100, the method can output an indication that there is a very low likelihood for severe outcome for scores between 0 and about 20, an indication that there is a low likelihood for severe outcome for scores between about 20 and about 40, an indication that there is a moderate likelihood for severe outcome for scores between about 40 and about 80, and an indication that there is a high likelihood for severe outcome for scores between about 80 and about 100.

In some embodiments of the present invention, the subject is treated (314) for the coronaviral infection based on the output generated at 313. For example, when the score is above a predetermined severity threshold, the subject can be treated by e.g., mechanical ventilation, life support, catheterization, hemofiltration, invasive monitoring, sedation, intensive care admission, surgical intervention, drug of last resort, and the like.

The method ends at 315.

### Example 9

RNAs that can be used to distinguish between a severe and non-severe infection or to distinguish between a viral and bacterial infection

| Gene symbol | RefSeq mRNA | Gene name |
|---|---|---|
| CEACAM8 | NM_001816.4 | CEA cell adhesion molecule 8 |
| FOLR3 | NM_000804.4 | folate receptor gamma |
| DEFA4 | NM_001925.3 | defensin alpha 4 |
| HIST1H4C | NM_003542.4 | H4 clustered histone 3 |
| BPI | NM_001725.3 | bactericidal permeability increasing protein |
| GPR84 | NM_020370.3 | G protein-coupled receptor 84 |
| LTF | NM_002343.6 | lactotransferrin |
| OLFM4 | NM_006418.5 | olfactomedin 4 |
| CPVL | NM_001371255.1 | carboxypeptidase vitellogenic like |
| TGFBI | NM_000358.3 | transforming growth factor beta induced |
| CECR1 | NM_001030767.2 | cat eye syndrome chromosome region, candidate 1 |
| IFIT2 | NM_001547.5 | interferon induced protein with tetratricopeptide |
| | | repeats 2 |
| CEACAM1 | NM_001712.5 | CEA cell adhesion molecule 1 |
| MTMR11 | NM_001145862.2 | myotubularin related protein 11 |
| C9orf95 | NM_017881.3 | nicotinamide riboside kinase 1 |
| GNA15 | NM_002068.4 | G protein subunit alpha 15 |
| BATF | NM_016767.2 | basic leucine zipper transcription factor, ATF-like |
| CD5 | NM_014207.4 | CD5 molecule |
| C3AR1 | NM_004054.4 | complement C3a receptor 1 |
| KIAA1370 | NM_019600.4 | family with sequence similarity 214 member A |
| TRIB1 | NM_025195.4 | tribbles pseudokinase 1 |
| MTCH1 | NM_001271641.2 | mitochondrial carrier 1 |
| CLEC10A | NM_182906.4 | C-type lectin domain containing 10A |
| RPGRIP1 | NM_020366.4 | RPGR interacting protein 1 |
| HLA-DPB1 | NM_002121.6 | major histocompatibility complex, class II, DP beta 1 |
| HK3 | NM_002115.3 | hexokinase 3 |
| CTSB | NM_001384714.1 | cathepsin B |
| GPAA1 | NM_003801.4 | glycosylphosphatidylinositol anchor attachment 1 |
| TNIP1 | NM_001252385.2 | TNFAIP3 interacting protein 1 |
| PLK1 | NM_005030.6 | polo like kinase 1 |
| JUP | NM_001352773.2 | junction plakoglobin |
| LAX1 | NM_017773.4 | lymphocyte transmembrane adaptor 1 |
| CX3CR1 | NM_001171174.1 | C-X3-C motif chemokine receptor 1 |
| CD163 | NM_004244.6 | CD163 molecule |
| CKS2 | NM_001827.3 | CDC28 protein kinase regulatory subunit 2 |
| RGS1 | NM_002926.4 | regulator of G protein signaling 12 |
| POLD3 | NM_006591.3 | DNA polymerase delta 3, accessory subunit |
| PER1 | NM_002616.3 | period circadian regulator 1 |
| HIF1A | NM_001530.4 | hypoxia inducible factor 1 subunit alpha |
| SEPP1 | NM_005410.4 | selenoprotein P |
| RCBTB2 | NM_001286830.2 | RCC1 and BTB domain containing protein 2 |
| CBFA2T3 | NM_005187.6 | CBFA2/RUNX1 partner transcriptional co-repressor 3 |
| C11orf74 | NM_001276722.2 | intraflagellar transport associated protein |
| CIT | NM_001206999.2 | citron rho-interacting serine/threonine kinase |
| DHRS7B | NM_015510.5 | dehydrogenase/reductase 7B |
| LY86 | NM_004271.4 | lymphocyte antigen 86 |
| MKI67 | NM_002417.5 | marker of proliferation Ki-67 |
| KCNJ2 | NM_000891.3 | potassium inwardly rectifying channel subfamily J |
| | | member 2 |
| CST3 | NM_001288614.2 | cystatin C |
| EMR3 | NM_032571.5 | adhesion G protein-coupled receptor E3 |
| IER3 | NM_003897.4 | immediate early response 3 |
| CD4 | NM_000616.5 | CD4 molecule |
| NAAA | NM_014435.4 | N-acylethanolamine acid amidase |
| CEP55 | NM_018131.5 | centrosomal protein 55 |
| UPB1 | NM_016327.3 | beta-ureidopropionase 1 |
| PRTN3 | NM_002777.4 | proteinase 3 |
| ELANE | NM_001972.4 | elastase, neutrophil expressed |
| GIMAP8 | NM_175571.4 | GTPase, IMAP family member 8 |
| TOP2A | NM_001067.4 | DNA topoisomerase II alpha |
| SLC7A7 | NM_001126105.3 | solute carrier family 7 member 7 |
| BMX | NM_203281.3 | BMX non-receptor tyrosine kinase |
| SLC7A5 | NM_003486.7 | solute carrier family 7 member 5 |
| CEACAM6 | NM_002483.7 | CEA cell adhesion molecule 6 |
| MPO | NM_000250.2 | myeloperoxidase |
| CD24 | NM_013230.3 | CD24 molecule |
| TFRC | NM_003234.4 | transferrin receptor |
| HLA-DRB1 | NM_002124.4 | major histocompatibility complex, class II, DR beta 1 |
| DEFA1B | NM_001302265.2 | defensin alpha 1B |
| TCN1 | NM_001062.4 | transcobalamin 1 |
| DEFA3 | NM_005217.4 | defensin alpha 3 |
| CCR3 | NM_001837.4 | C-C motif chemokine receptor 3 |
| FGFBP2 | NM_031950.4 | fibroblast growth factor binding protein 2 |
| CTSG | NM_001911.3 | cathepsin G |
| IL7R | NM_002185.5 | interleukin 7 receptor |
| TYMS | NM_001071.4 | thymidylate synthetase |
| DEFA1 | NM_004084.4 | defensin alpha 1 |
| CLEC7A | NM_197947.3 | C-type lectin domain containing 7A |
| SIAH2 | NM_005067.7 | siah E3 ubiquitin protein ligase 2 |
| YOD1 | NM_018566.4 | YOD1 deubiquitinase |
| RAP1GAP | NM_001330383.3 | RAP1 GTPase activating protein |
| ZBP1 | NM_030776.3 | Z-DNA binding protein 1 |
| KIAA1324 | NM_020775.5 | endosome-lysosome associated apoptosis and |
| | | autophagy regulator 1 |
| CA1 | NM_001128830.4 | carbonic anhydrase 1 |
| TST | NM_003312.6 | thiosulfate sulfurtransferase |
| OR52R1 | NM_001005177.3 | olfactory receptor family 52 subfamily R member 1 |
| TDRD9 | NM_153046.3 | tudor domain containing 9 |
| GPBAR1 | NM_001077191.2 | G protein-coupled bile acid receptor 1 |
| LGALS2 | NM_006498.3 | galectin 2 |
| CLIC3 | NM_004669.3 | chloride intracellular channel 3 |
| PRF1 | NM_001083116.3 | perforin 1 |
| IL1R2 | NM_004633.4 | interleukin 1 receptor type 2 |
| FCER1A | NM_001387280.1 | Fc fragment of IgE receptor 1a |
| HES4 | NM_001142467.2 | hes family bHLH transcription factor 4 |
| LRRN3 | NM_001099660.2 | leucine rich repeat neuronal 3 |
| TPST1 | NM_003596.4 | tyrosylprotein sulfotransferase 1 |
| TMCC2 | NM_014858.4 | transmembrane and coiled-coil domain family 2 |
| ANKRD22 | NM_144590.3 | ankyrin repeat domain 22 |
| OLAH | NM_018324.3 | oleoyl-ACP hydrolase |
| PFKFB2 | NM_006212.2 | 6-phosphofructo-2-kinase/fructose-2,6-biphosphatase 2 |
| GRB10 | NM_001371009.1 | growth factor receptor bound protein 10 |
| ASPH | NM_004318.4 | aspartate beta-hydroxylase |
| LRRC70 | NM_181506.5 | leucine rich repeat containing 70 |
| IL1R1 | NM_000877.4 | interleukin 1 receptor type 1 |
| IRAK3 | NM_007199.3 | interleukin 1 receptor associated kinase 3 |
| NSUN7 | NM_024677.6 | NOP2/Sun RNA methyltransferase family member 7 |
| UGCG | NM_003358.3 | UDP-glucose ceramide glucosyltransferase |
| GADD45A | NM_001924.4 | growth arrest and DNA damage inducible alpha |
| ANXA3 | NM_005139.3 | annexin A3 |
| ST6GALNAC3 | NM_152996.4 | ST6 N-acetylgalactosaminide alpha-2,6- |
| | | sialyltransferase 3 |
| SRPK1 | NM_003137.5 | SRSF protein kinase 1 |
| PYGL | NM_002863.5 | glycogen phosphorylase L |
| PCOLCE2 | NM_013363.4 | procollagen C-endopeptidase enhancer 2 |
| MGAM | NM_001365693.1 | maltase-glucoamylase |
| CYSTM1 | NM_032412.4 | cysteine rich transmembrane module containing 1 |
| GNLY | NM_001302758.2 | granulysin |
| HLA-DPA1 | NM_033554.3 | major histocompatibility complex, class II, DP alpha 1 |
| ZAP70 | NM_001079.4 | zeta chain of T cell receptor associated protein kinase 70 |
| PTPRCAP | NM_005608.3 | protein tyrosine phosphatase receptor type C |
| | | associated protein |
| CD6 | NM_001780.6 | CD63 molecule |
| XCL2 | NM_003175.4 | X-C motif chemokine ligand 2 |
| GIMAP5 | NM_018384.5 | GTPase, IMAP family member 5 |
| HLA-DQB1 | NM_002123.5 | major histocompatibility complex, class II, DQ beta 1 |
| CD3D | NM_000732.6 | CD3d molecule |
| IL32 | NM_001012631.4 | interleukin 32 |
| GZMM | NM_005317.4 | granzyme M |
| CD2 | NM_001328609.2 | CD2 molecule |
| GZMB | NM_004131.6 | granzyme B |
| MAL | NM_002371.4 | mal, T cell differentiation protein |
| LCK | NM_001042771.3 | LCK proto-oncogene, Src family tyrosine kinase |
| PID1 | NM_017933.5 | phosphotyrosine interaction domain containing 1 |
| CD160 | NM_007053.4 | CD160 molecule |
| SIGIRR | NM_001135054.2 | single Ig and TIR domain containing |
| CD52 | NM_001803.3 | CD52 molecule |
| GZMH | NM_033423.5 | granzyme H |
| TNFRSF25 | NM_148965.2 | TNF receptor superfamily member 25 |
| CCR5 | NM_000579.4 | C-C motif chemokine receptor 5 |
| C12orf57 | NM_138425.4 | chromosome 12 open reading frame 57 |
| CD247 | NM_198053.3 | CD247 molecule |
| CCR7 | NM_001838.4 | C-C motif chemokine receptor 7 |
| CD7 | NM_001025159.2 | CD74 molecule |
| MATK | NM_139355.3 | megakaryocyte-associated tyrosine kinase |
| RPL36 | NM_033643.3 | ribosomal protein L36 |
| FLT3LG | NM_001204502.2 | fms related receptor tyrosine kinase 3 ligand |
| SIRPG | NM_018556.4 | signal regulatory protein gamma |
| PLAAT4 | NM_004585.5 | phospholipase A and acyltransferase 4 |
| RPS28 | NM_001031.5 | ribosomal protein S28 |
| TBX21 | NM_013351.2 | T-box transcription factor 21 |
| TRABD2A | NM_001277053.2 | TraB domain containing 2A |
| LFNG | NM_001040167.2 | LFNG O-fucosylpeptide 3-beta-N- |
| | | acetylglucosaminyltransferase |
| SPOCK2 | NM_014767.2 | SPARC (osteonectin), cwcv and kazal like domains |
| | | proteoglycan 2 |
| NCR3 | NM_147130.3 | natural cytotoxicity triggering receptor 3 |
| CD3E | NM_000733.4 | CD3e molecule |
| CYB561 | NM_001017916.2 | cytochrome b561 |
| RPS19 | NM_001321483.2 | ribosomal protein S19 |
| HLA-DRA | NM_019111.5 | major histocompatibility complex, class II, DR alpha |
| HLA-DMB | NM_002118.5 | major histocompatibility complex, class II, DM beta |
| TLE5 | NM_198969.1 | TLE family member 5, transcriptional modulator |
| MAD1L1 | NM_003550.3 | mitotic arrest deficient 1 like 1 |
| SAMSN1 | NM_022136.5 | SAM domain, SH3 domain and nuclear localization |
| | | signals 1 |
| PRC1 | NM_003981.4 | protein regulator of cytokinesis 1 |
| RETN | NM_001385726.1 | resistin |
| CD74 | NM_001025159.2 | CD74 molecule |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

Arias, C. A., and B. E. Murray. 2009. “Antibiotic-Resistant Bugs in the 21st Century—a Clinical Super-Challenge.” The New England Journal of Medicine 360 (5): 439-43. doi:10.1056/NEJMp0804651.
Bogaert, D, R De Groot, and P W M Hermans. 2004. “Streptococcus Pneumoniae Colonisation: The Key to Pneumococcal Disease.” The Lancet Infectious Diseases 4 (3): 144-54. doi:10.1016/S1473-3099(04)00938-7.
Bossuyt, Patrick M, Johannes B Reitsma, David E Bruns, Constantine A Gatsonis, Paul P Glasziou, Les M Irwig, David Moher, Drummond Rennie, Henrica C. W De Vet, and Jeroen G Lijmer. 2003. “The STARD Statement for Reporting Studies of Diagnostic Accuracy: Explanation and Elaboration.” Annals of Internal Medicine 138 (1): W1-12.
Cadieux, G., and R. Tamblyn, et al. 2007. “Predictors of Inappropriate Antibiotic Prescribing among Primary Care Physicians.” CMAJ. Canadian Medical Association Journal=Journal De l'Association Medicale Canadienne 177 (8): 877-83.
“CDC—About Antimicrobial Resistance.” 2013. Accessed January 17. www(dot)cdc(dot)gov/drugresistance/about(dot)html.
“CDC—Get Smart: Fast Facts About Antibiotic Resistance.” 2011. www(dot)cdc(dot)gov/getsmart/antibiotic-use/fast-facts(dot)html.
______. 2013. Accessed January 17. www(dot)cdc(dot)gov/getsmart/antibiotic-use/fast-facts(dot)html.
Cohen, Asi, Louis Bont, Dan Engelhard, Edward Moore, David Fernández, Racheli Kreisberg-Greenblatt, Kfir Oved, Eran Eden, and John P. Hays. 2015. “A Multifaceted ‘Omics’ Approach for Addressing the Challenge of Antimicrobial Resistance.” Future Microbiology 10 (3): 365-76. doi: 10.2217/fmb.14.127.
Davey, P., and E. Brown, et al. 2006. “Systematic Review of Antimicrobial Drug Prescribing in Hospitals.” Emerging Infectious Diseases 12 (2): 211-16.
Del Mar, C. 1992. “Managing Sore Throat: A Literature Review. I. Making the Diagnosis.” The Medical Journal of Australia 156 (8): 572-75.
Downey, Tom. 2006. “Analysis of a Multifactor Microarray Study Using Partek Genomics Solution.” Methods in Enzymology 411: 256-70. doi:10.1016/50076-6879(06)11013-7.
Engel, Madelon F, F P Paling, A I M Hoepelman, V van der Meer, and J J Oosterheert. 2012. “Evaluating the Evidence for the Implementation of C-Reactive Protein Measurement in Adult Patients with Suspected Lower Respiratory Tract Infection in Primary Care: A Systematic Review.” Family Practice 29 (4): 383-93. doi:10.1093/fampra/cmr119.
“European Surveillance of Antimicrobial Consumption Network (ESAC-Net).” 2014. Accessed February 26. www(dot)ecdc(dot)europa(dot)eu/en/activities/surveillance/ESAC-Net/Pages/index(dot)aspx.
Falk, Gavin, and Tom Fahey. 2009. “C-Reactive Protein and Community-Acquired Pneumonia in Ambulatory Care: Systematic Review of Diagnostic Accuracy Studies.” Family Practice 26 (1): 10-21. doi:10.1093/fampra/cmn095.
Houck, P. M., and D. W. Bratzler, et al. 2002. “Pneumonia Treatment Process and Quality.” Archives of Internal Medicine 162 (7): 843-44.
Jung, C. L., M. A. Lee, and W. S. Chung. 2010. “Clinical Evaluation of the Multiplex PCR Assay for the Detection of Bacterial Pathogens in Respiratory Specimens from Patients with Pneumonia.” Korean Journal of Clinical Microbiology 13 (1): 40. doi:10.5145/KJCM.2010.13.1.40.
Kim, K. H., J. H. Shin, and S. Y. Kim. 2009. “The Clinical Significance of Nasopharyngeal Carriages in Immunocompromised Children as Assessed.” The Korean Journal of Hematology 44 (4): 220. doi:10.5045/kjh.2009.44.4.220.
Limper, M., M. D. de Kruif, A. J. Duits, D. P. M. Brandjes, and E. C. M. van Gorp. 2010. “The Diagnostic Role of Procalcitonin and Other Biomarkers in Discriminating Infectious from Non-Infectious Fever.” Journal of Infection 60 (6): 409-16. doi:10.1016/j.jinf.2010.03.016.
Linder, J A, and R S Stafford. 2001. “Antibiotic Treatment of Adults with Sore Throat by Community Primary Care Physicians: A National Survey, 1989-1999.” JAMA: The Journal of the American Medical Association 286 (10): 1181-86.
Little, P. 2005. “Delayed Prescribing of Antibiotics for Upper Respiratory Tract Infection.” BMJ (Clinical Research Ed.) 331 (7512): 301-2.
Little, P. S., and I. Williamson. 1994. “Are Antibiotics Appropriate for Sore Throats? Costs Outweigh the Benefits.” BMJ (Clinical Research Ed.) 309 (6960): 1010-11.
Oved, Kfir, Asi Cohen, Olga Boico, Roy Navon, Tom Friedman, Liat Etshtein, Or Kriger, et al. 2015. “A Novel Host-Proteome Signature for Distinguishing between Acute Bacterial and Viral Infections.” PLoS ONE 10 (3): e0120012. doi:10.1371/journal.pone.0120012.
Pulcini, C., and E. Cua, et al. 2007. “Antibiotic Misuse: A Prospective Clinical Audit in a French University Hospital.” European Journal of Clinical Microbiology & Infectious Diseases: Official Publication of the European Society of Clinical Microbiology 26 (4): 277-80.
Quenot, Jean-Pierre, Charles-Edouard Luyt, Nicolas Roche, Martin Chalumeau, Pierre-Emmanuel Charles, Yann-Eric Claessens, Sigismond Lasocki, et al. 2013. “Role of Biomarkers in the Management of Antibiotic Therapy: An Expert Panel Review II: Clinical Use of Biomarkers for Initiation or Discontinuation of Antibiotic Therapy.” Annals of Intensive Care 3 (July): 21. doi:10.1186/2110-5820-3-21.
Ramilo, Octavio, Windy Allman, Wendy Chung, Asuncion Mejias, Monica Ardura, Casey Glaser, Knut M Wittkowski, et al. 2007. “Gene Expression Patterns in Blood Leukocytes Discriminate Patients with Acute Infections.” Blood 109 (5): 2066-77. doi:10.1182/blood-2006-02-002477.
Rhedin, Samuel, Ann Lindstrand, Maria Rotzen-Ostlund, Thomas Tolfvenstam, Lars Ohrmalm,
Malin Ryd Rinder, Benita Zweygberg-Wirgart, et al. 2014. “Clinical Utility of PCR for Common Viruses in Acute Respiratory Illness.” Pediatrics, February, peds.2013-3042. doi: 10.1542/peds 0.2013-3042.
Scott, J. G., and D. Cohen. 2001. “Antibiotic Use in Acute Respiratory Infections and the Ways Patients Pressure Physicians for a Prescription.” The Journal of Family Practice 50 (10): 853-58.
Shin, J. H., H. Y. Han, and S. Y. Kim. 2009. “Detection of Nasopharyngeal Carriages in Children by Multiplex Reverse Transcriptase-Polymerase Chain Reaction.” Korean Journal of Pediatrics 52 (12): 1358. doi:10.3345/kjp.2009.52.12.1358.
Spiro, D. M., and K. Y. Tay, et al. 2006. “Wait-and-See Prescription for the Treatment of Acute Otitis Media: A Randomized Controlled Trial.” JAMA: The Journal of the American Medical Association 296 (10): 1235-41.
Spuesens, Emiel B. M., Pieter L. A. Fraaij, Eline G. Visser, Theo Hoogenboezem, Wim C. J. Hop, Léon N. A. van Adrichem, Frank Weber, et al. 2013. “Carriage of Mycoplasma Pneumoniae in the Upper Respiratory Tract of Symptomatic and Asymptomatic Children: An Observational Study.” PLoS Med 10 (5): e1001444. doi:10.1371/journal.pmed.1001444.
Tang, Benjamin M P, Guy D Eslick, Jonathan C Craig, and Anthony S McLean. 2007. “Accuracy of Procalcitonin for Sepsis Diagnosis in Critically Ill Patients: Systematic Review and Meta-Analysis.” The Lancet Infectious Diseases 7 (3): 210-17. doi:10.1016/S1473-3099(07)70052-X.
“Threat Report 2013|Antimicrobial Resistance|CDC.” 2013. Accessed November 10. www(dot)cdc(dot)gov/drugresistance/threat-report-2013/.
Tian, Qiang, Serguei B. Stepaniants, Mao Mao, Lee Weng, Megan C. Feetham, Michelle J. Doyle,
Eugene C. Yi, et al. 2004. “Integrated Genomic and Proteomic Analyses of Gene Expression in Mammalian Cells.” Molecular & Cellular Proteomics: MCP 3 (10): 960-69. doi:10.1074/mcp.M400055-MCP200.
Uyeki, Timothy M, Ramakrishna Prasad, Charles Vukotich, Samuel Stebbins, Charles R Rinaldo, Yu-Hui Ferng, Stephen S Morse, et al. 2009. “Low Sensitivity of Rapid Diagnostic Test for Influenza.” Clinical Infectious Diseases: An Official Publication of the Infectious Diseases Society of America 48 (9): e89-92. doi:10.1086/597828.
van der Meer, Victor, Arie Knuistingh Neven, Peterhans J van den Broek, and Willem J J Assendelft. 2005. “Diagnostic Value of C Reactive Protein in Infections of the Lower Respiratory Tract: Systematic Review.” BMJ (Clinical Research Ed.) 331 (7507): 26. doi:10.1136/bmj.38483.478183.EB.
“WHO|Antimicrobial Resistance.” 2013. Accessed December 5. www(dot)who(dot)int/mediacentre/factsheets/fs194/en/index(dot)html.
“WHO Europe—Data and Statistics.” 2014. Accessed February 24. /www(dot)euro(dot)who(dot)int/en/health-topic s/disease-prevention/antimicrobial-resistance/data-and-statistics.

1. A method of treating an infection in a test subject comprising:
(a) measuring the amount of CD177 RNA and the amount of IFI44L RNA in a sample obtained from the subject;
(b) generating a score based on said amount of CD177 RNA and said amount of IFI44L RNA, wherein the score is an increasing function of said amount of CD177 RNA and a decreasing function of said amount of IFI44L RNA, wherein:
(i) when the score is below a first predetermined level, the subject is treated with an antiviral agent, wherein the first predetermined level is based on the amount of CD177 RNA and the amount of IFI44L RNA in bacterial subjects;
(ii) when the score is above a second predetermined level, the subject is treated with an antibiotic, wherein the second predetermined level is based on the amount of CD177 RNA and the amount of IFI44L RNA in viral subjects; and/or
(iii) when the score is above a third predetermined level, the subject is treated with an antiviral agent, wherein the third predetermined level is based on the amount of CD177 RNA and the amount of IFI44L RNA in non-infectious subjects.

2-4. (canceled)

5. The method of claim 1, wherein the function is a linear function.

6. The method of claim 1, further comprising measuring the amount of at least one additional RNA selected from the group consisting of MMP9 RNA, IFIT1 RNA and PI3 RNA in a sample of the subject.

7. The method of claim 1, further comprising measuring the amount of IFIT1 RNA in a sample of the subject.

8. The method of claim 1, further comprising measuring the amount of each of MMP9 RNA, IFIT1 RNA and PI3 RNA in a sample of the subject.

9. The method of claim 8, when the score is based on the amount of said CD177 RNA said IFI44L RNA said IFIT1 RNA said MMP9 RNA and said PI3 RNA.

10-23. (canceled)

24. The method of claim 1, wherein the test subject does not have a chronic disease.

25. The method of claim 1, wherein the test subject shows symptoms of an infection.

26. The method of claim 25, wherein said symptoms are selected from the group consisting of fever, cough, sputum production, myalgia, fatigue, headache, anorexia, dyspnea, diarrhea, nausea, dizziness, headache, vomiting, abdominal pain, sore throat, nasal congestion, hemoptysis and chills.

27. The method of claim 1, wherein the test subject is asymptomatic of an infection.

28-29. (canceled)

30. The method of claim 1, wherein the level of no more than 10 RNA markers is used to determine the infection type.

31-55. (canceled)

56. A method of identifying an infection outbreak or a change in virulence of an existing pathogen in a medical facility, the method comprising:
obtaining a distribution pertaining to expression values of TRAIL protein, IP10 protein, and CRP protein derived from each of a plurality of patients in the medical facility;
accessing a computer readable medium storing comparative data;
comparing said calculated distribution to said comparative data; and
issuing an alert that an infection is expected to outbreak across the medical facility, or that a change in virulence of the existing pathogen in the medical facility is expected to occur, when said comparison indicates a rise in said distribution above a predetermined threshold, and issuing a report pertaining to said comparison otherwise.

57. (canceled)

58. The method according to claim 56, comprising receiving said expression values of said proteins, and wherein said obtaining comprises calculating said distribution.

59. The method according to claim 56, wherein said distribution pertaining to said expression values is a distribution of a classification score calculated based on said expression values.

60. The method according to claim 59, comprising calculating said classification score.

61. The method according to claim 56, further comprising obtaining a distribution pertaining to expression values of at least one additional determinant selected from the group consisting of determinants listed in Table 34, wherein said accessing said computer readable medium and said comparing is also executed with respect to said at least one additional determinant.

62-63. (canceled)

64. The method according to claim 56, wherein said comparative data comprises history data pertaining to previously received expression values of said proteins within the medical facility.

65. The method according to claim 56, being executed at a central facility remote to the medical facility, wherein the method comprises transmitting said alert to a receiving computer at the medical facility.

66. The method according to claim 65, being executed for a plurality of medical facilities, wherein said transmitting is separate to each medical facility.

67-68. (canceled)

69. The method according to claim 56, comprising applying quarantine to said medical facility upon determination that an infection is expected to outbreak across the medical facility.
